SEQUENCE LISTING (22 sequences)

SEQ ID NO: 1 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTGPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 2 - DNA, 954 bp, Homo sapiens
  source 1..954: mol_type = genomic DNA
atgactatggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 3 - DNA, 349 bp, synthetic construct
  source 1..349: mol_type = other DNA
gaggtgcagctgcaggagtctggacctgagctggtgaagcctggggcttcagtgaagatatcctgcaaggcttctggctacaccttcacaacctactatatacactgggtgaagcagaggcctggacagggacttgagtggattggatggatttttcctggaagtagtaatactttctacaatgagaaattcaagggcaaggccacactgacggcagacacatccgccatcactgcctacatgcagctcaacagcctgacatctgaagactctgcagtctatttctgttcaagatattactacgactatggttactggggccaagggactctggtcactgtctctgcag

SEQ ID NO: 4 - protein, 116 aa, synthetic construct
EVQLQESGPELVKPGASVKISCKASGYTFTTYYIHWVKQRPGQGLEWIGWIFPGSSNTFYNEKFKGKATLTADTSAITAYMQLNSLTSEDSAVYFCSRYYYDYGYWGQGTLVTVSA

SEQ ID NO: 5 - protein, 8 aa, synthetic construct
GYTFTTYY

SEQ ID NO: 6 - protein, 8 aa, synthetic construct
IFPGSSNT

SEQ ID NO: 7 - protein, 9 aa, synthetic construct
SRYYYDYGY

SEQ ID NO: 8 - DNA, 319 bp, synthetic construct
  source 1..319: mol_type = other DNA
gaaaatgttctcacccagtctccagcaatcatgtctgcatctccaggggaaaaggtcaccatgacctgcagtgccagctcaagtgttcgttacatgcaatggtaccagcagaagtcaaacacctcccccaaactctggatttatgacacatccaaactggcttctggagtcccaggtcgcttcagtggcagtgggtctggaaactcttactttctcacgatcagcagcatggaggctgaagatgttgccacttatcactgttttcaggggagtggttacccactcacgttcggaggggggaccaagctggaaataaaac

SEQ ID NO: 9 - protein, 106 aa, synthetic construct
ENVLTQSPAIMSASPGEKVTMTCSASSSVRYMQWYQQKSNTSPKLWIYDTSKLASGVPGRFSGSGSGNSYFLTISSMEAEDVATYHCFQGSGYPLTFGGGTKLEIK

SEQ ID NO: 10 - protein, 5 aa, synthetic construct
SSVRY

SEQ ID NO: 11 - skipped

SEQ ID NO: 12 - protein, 9 aa, synthetic construct
FQGSGYPLT

SEQ ID NO: 13 - DNA, 349 bp, synthetic construct
  source 1..349: mol_type = other DNA
gaggtgcagctgcaggagtctggacctgagctggtgaagcctggggcttcagtgaggatttcctgcaaggcctctgggtacaccttcacaaactcctatatgcactgggtgaagcagaggcctggacagggacttgagtggattggatggatttatcctgaaaatattaatacttattacaatgagaagttcaagggcaaggccacactgactgcagacagatcctccagcacagcctacatgcagctcaacagcctggcctctgaggactctgcggtctatttctgtgcaagattttattacggcttcggctactggggccaaggcaccactctcacagtctcctcag

SEQ ID NO: 14 - protein, 116 aa, synthetic construct
EVQLQESGPELVKPGASVRISCKASGYTFTNSYMHWVKQRPGQGLEWIGWIYPENINTYYNEKFKGKATLTADRSSSTAYMQLNSLASEDSAVYFCARFYYGFGYWGQGTTLTVSS

SEQ ID NO: 15 - protein, 8 aa, synthetic construct
GYTFTNSY

SEQ ID NO: 16 - protein, 8 aa, synthetic construct
IYPENINT

SEQ ID NO: 17 - protein, 9 aa, synthetic construct
ARFYYGFGY

SEQ ID NO: 18 - DNA, 322 bp, synthetic construct
  source 1..322: mol_type = other DNA
gacattgtgatgacccagtctcacaagttcatgtccacatcagtgggagacagggtcatcatcacctgcaaggccagtcagaatgtgatttctgctgtagcctggtatcaacagaaaccaggacattctcctaaactactgatttattgggcatccacccggcacactggagtccctgatcgcttcacaggcagtggatctgggacagatttcactctcaccattaacagtgtgcagtctgaggacttggcagattatttctgtcagcaatatggcaggtatccttacacgttcgcaggggggaccaagctggaaataaaac

SEQ ID NO: 19 - protein, 107 aa, synthetic construct
DIVMTQSHKFMSTSVGDRVIITCKASQNVISAVAWYQQKPGHSPKLLIYWASTRHTGVPDRFTGSGSGTDFTLTINSVQSEDLADYFCQQYGRYPYTFAGGTKLEIK

SEQ ID NO: 20 - protein, 6 aa, synthetic construct
QNVISA

SEQ ID NO: 21 - skipped

SEQ ID NO: 22 - protein, 9 aa, synthetic construct
QQYGRYPYT

(19) United States
(12) Patent Application Publication
(10) Pub. No.: US 2026/0055190 A1
(43) Pub. Date: Feb. 26, 2026

(54) ANTIBODIES FOR PLATELET FCyRIIA AND RELATED METHODS OF USE

(71) Applicant: UNIVERSITY OF VERMONT AND STATE AGRICULTURAL COLLEGE, Burlington, VT (US)

(72) Inventor: David SCHNEIDER, Burlington, VT (US)

(21) Appl. No.: 19/104,163

(22) PCT Filed: Aug. 16, 2023
(86) PCT No.: PCT/US2023/072306

§ 371 Date: Feb. 14, 2025

Related U.S. Application Data
(60) Provisional application No. 63/371,636, filed on Aug. 16, 2022.

(51) Int. Cl. C07K 16/28; G01N 1/30; G01N 33/68

(52) U.S. Cl. CPC C07K 16/283; G01N 1/30; G01N 33/6854; C07K 2317/76; G01N 2333/70535

(57) ABSTRACT

Antibodies that interact with FcγRIIa are described. Methods of detecting the amount of FcγRIIa on platelets in a sample using antibodies to FcγRIIa are described. Methods of treating a selected subject at risk of thrombosis with an anti-thrombotic therapy are described involving administering aspirin, cilostazol or an anti-thrombotic agent that is an Adenosine diphosphate (ADP) receptor antagonist and/or a Protease-activated receptor (PAR) antagonist to the selected subject, wherein the subject is selected by determining a level of FcγRIIa expressed on platelets by detecting the amount of FcγRIIa in a sample using antibodies to FcγRIIa. In certain methods, elevated levels of FcγRIIa expression identify the subject as at risk of thrombosis and in need for anti-thrombotic therapy.

### CROSS-REFERENCE TO RELATED APPLICATIONS

This application claims priority to U.S. Provisional Application No. 63/371,636 filed on Aug. 16, 2022, the content of which is incorporated by reference herein in its entirety.

### REFERENCE TO AN ELECTRONIC SEQUENCE LISTING

The contents of the electronic sequence listing (179429.00023.xml; Size: 21,565 bytes; and Date of Creation: Aug. 15, 2023) is herein incorporated by reference in its entirety.

### BACKGROUND

Increased platelet reactivity contributes to a greater risk of thrombosis, the proximate cause of heart attack and stroke. Given the negative health effects of thrombosis, assays for platelet reactivity should be useful for identifying individuals who are at risk of thrombosis, and in selecting appropriate therapeutic regimens. However, current assays of platelet reactivity have not demonstrated that capacity and are sensitive to medications and other therapies which are in common use. Therefore, novel assays for platelet reactivity and related innovation, techniques, and medications are needed.

### SUMMARY

As described below, the present disclosure features antibodies that bind to FcγRIIa which are useful in assaying platelet reactivity, along with assays featuring the use of these antibodies. Disclosed herein are monoclonal antibodies able to recognize and bind to FcγRIIa. The monoclonal antibodies are able to bind to and interfere with, modulate, and/or inhibit the binding interactions between FcγRIIa and other molecules.

Also disclosed are further compositions and methods for quantifying FcγRIIa on platelets and thereby assaying platelet reactivity. Platelet reactivity is a powerful marker of medical risk but to date, available tests of platelet reactivity are sensitive to external factors and reproducibility concerns and have not proven useful as indicators of medical risk. In general, a blood sample is removed from an individual and a suitable anticoagulant added to the in vitro sample. Platelets in the sample are then fixed by adding a suitable fixative. Subsequently an antibody that binds to FcγRIIa (e.g., a primary antibody conjugated with a detectable label) and/or a secondary conjugated antibody is added to the sample. The sample may be analyzed with the use of flow cytometry. Platelets are identified by their characteristic size and the mean fluorescence intensity reflecting the surface expression of FcγRIIa may be quantified. In addition to mean fluorescence, a quantitative measurement, for instance, the number of copies of FcγRIIa, instance, FcγRIIa molecules per platelet, or an increase in level over prior levels or over a reference level may be determined. Expression of FcγRIIa at or above a predefined threshold may be used to identify patients with elevated expression of FcγRIIa and increased platelet reactivity.

In one aspect, the method is based on quantification of the surface protein FcγRIIa on platelets without activation of the platelets. In another aspect, the method may be performed on fixed platelets. In another aspect, the method avoids the results being influenced by assay conditions (e.g. the anticoagulants used during phlebotomy) and/or patient treatment. In another aspect, the method is performed such that neither anticoagulants nor a P2Y12 antagonist alters platelet expression of FcγRIIa and therefor does not alter test results.

In one aspect, a method of identifying a subject (e.g., human) with increased platelet reactivity is disclosed involving determining a level of FcγRIIa on platelets from the subject; and comparing the level of FcγRIIa on the platelets with a reference value, where an increased level compared to the reference value indicates that the subject has increased platelet reactivity. The level of FcγRIIa on platelets may be determined as a mean fluorescence intensity (MFI) measurement. Additionally, an MFI measurement maybe be standardized and converted into a measurement of the approximate or actual number of copies of FcγRIIa on the platelets in the sample.

In another aspect, a method of identifying a subject (e.g., human) having an increased risk of thrombosis is disclosed involving determining a level of FcγRIIa expressed on platelets from the subject; and comparing the level of FcγRIIa on the platelets with a reference value, where an increased level compared to the reference indicates that the subject has an increased risk of thrombosis.

In yet another aspect, a method of determining platelet reactivity is disclosed involving determining a level of FcγRIIa expressed on platelets from a subject, and comparing the level of FcγRIIa on the platelets with a reference value where an increased level compared to the reference is indicative of increased platelet reactivity.

In yet another aspect, a method of selecting anti-thrombotic therapy in a subject is disclosed involving determining a level of FcγRIIa expressed on platelets from the subject, and comparing the level of FcγRIIa to a reference value where an increased level compared to the reference value is indicative of a need for preventive therapy, anti-thrombotic therapy or additional anti-thrombotic therapy. In one embodiment, the anti-thrombotic therapy is one or more medications selected from the group consisting of aspirin, cilostazol, prasugrel, ticagrelor, clopidogrel, and vorapaxar.

In yet another aspect, a kit is disclosed for determining platelet reactivity containing an FcγRIIa specific reagent and instructions for use of the kit in the method of any of the above-aspects.

In yet another aspect, a method is disclosed for inhibiting platelet activation involving administering to a subject in need thereof an effective amount of an agent that inhibits FcγRIIa activation, thereby inhibiting platelet activation. In one embodiment, the agent is any one or more of a small molecule, an inhibitory nucleic acid, and an antibody or antigen-binding fragment thereof. In another embodiment, the inhibitory nucleic acid is any one or more of an antisense molecule, an shRNA, and an siRNA. In one embodiment, the inhibitory nucleic acid reduces the levels of FcγRIIa in megakaryoctes. In another embodiment, the subject is determined to be in need if platelets obtained from the subject have increased levels of FcγRIIa compared to a reference value.

In yet another aspect, a test device is disclosed for detecting FcγRIIa in a liquid sample, the device having a liquid permeable material defining the following portions in capillary communication: a) a first portion that is the site for application of a liquid sample, including a liquid permeable medium, and an FcγRIIa-binding conjugate; b) a second portion including a liquid permeable medium; and c) a third portion that is the site for detecting the binding of the FcγRIIa-binding conjugate at the test site, the third portion including a liquid permeable medium having the FcγRIIa fixed to the medium at the test site.

In a related aspect, a method is disclosed for determining platelet reactivity involving: determining a level of FcγRIIa expressed on platelets using a test device of the disclosure, and comparing the level of FcγRIIa on the platelets with a reference value where an increased level compared to the reference is indicative of increased platelet reactivity. In another related aspect, the disclosure provides a method for detecting FcγRIIa in a liquid sample, the method involving: a) applying a liquid sample to a device of the disclosure; and b) detecting presence or absence of an FcγRIIa-binding conjugate at a test site, where the absence of the FcγRIIa-binding conjugate at the test site identifies the presence of FcγRIIa in the sample and the presence of FcγRIIa-binding conjugate at the test site identifies the absence of the FcγRIIa in the sample.

In a related aspect, a kit is disclosed, comprising a test device according to the disclosure. In various embodiments, the kit includes instructions for the use of the device for the detection of an analyte. In other embodiments, the kit includes a means for measuring a liquid sample and a test vial.

In yet another aspect, a composition or kit is provided for identifying and treating a subject having increased platelet reactivity, the composition including an FcγRIIa specific reagent and directions for using the reagent to measure the level of FcγRIIa in a biological sample of a subject, where a level greater than about 7,500 copies of FcγRIIa per platelet identifies the subject as having increased platelet reactivity; and (b) a therapeutic reagent that is one or more of prasugrel, ticagrelor, clopidogrel, and vorapaxar.

In various embodiments of the above-aspects or any other aspect of the disclosure provided herein, the reference value is a measured or expected level of FcγRIIa on the surface of platelets from a disease-free, low-risk, or otherwise healthy individual. In one embodiment the reference value is at or about 1,500 copies of FcγRIIa per platelet and the increased level is at or about 1,600, 1,700, 1,800, 1,900, 2,000, 2,100, 2,200, 2,300, 2,400 or 2,500 copies of FcγRIIa per platelet, or more. The increased level may be any level above 1,600, 1,700, 1,800, 1,900, 2,000, 2,100, 2,200, 2,300, 2,400 or 2,500 copies of FcγRIIa per platelet, or more. In another embodiment, the level of FcγRIIa is determined using an FcγRIIa specific reagent. In another embodiment, the FcγRIIa specific reagent is an antibody or antigen-binding fragment thereof. In another embodiment, the level of platelet FcγRIIa is determined using an assay selected from the group consisting of flow cytometry, immunoassay, ELISA, western blotting, and radioimmunoassay. In another embodiment, the level of FcγRIIa is determined using fluorometric or colorimetric assay. In still other embodiments, the level of FcγRIIa is determined using flow cytometry.

In still other embodiments, the reference value represents an average level of FcγRIIa on platelets from a set of disease-free, low-risk, or otherwise healthy subjects. The increased level may be indicated by a measured level in a patient that is increased by at least about 1.5 fold, 1.6, 1.7, 1.8, 1.9, 2, 2.1, 2.2, 2.3, 2.4, and 2.5 fold or more, to include 5-10-fold, or 10-25 fold, as compared to the reference value determined based on an average value of disease-free, low-risk, or otherwise healthy patients. In various embodiments, the level of FcγRIIa is determined using an FcγRIIa specific reagent. In particular embodiments, the FcγRIIa specific reagent or FcγRIIa-binding conjugate is an antibody or antigen-binding fragment thereof. In various embodiments, the anti-thrombotic therapy is one or more of aspirin, cilostazol, prasugrel, ticagrelor, clopidogrel, and vorapaxar, or combinations thereof.

The reference value for the set of disease-free, low-risk, or otherwise healthy subjects may be determined by measuring, for instance, levels of FcγRIIa as MFI, numbers of copies of FcγRIIa, or otherwise, and determining an average for a set of for instance, 2-50, 5-30, 10-20, or at least 5, or at least 10, 15, 20, or 25 subjects, up to and including ranges of 30, 40, 50 or more, who have medical histories indicating that they are disease-free, low-risk, or otherwise healthy subjects, including those whose histories are free from disease, including cardiac disease, cardiac events, or cardiac therapy, or otherwise are free from indications that are or might be related to an increased risk of future cardiac events including stroke and thrombosis. In one embodiment a low-risk subject is one exhibiting no detectable disease state. In another embodiment a low-risk subject is one that has had no more than one known cardiac event, including a heart attack.

In various embodiments of any of the aspects delineated herein, the first portion of the test device further contains a control conjugate; and the third portion of the test device contains a control conjugate binder present at a control site for detecting the binding of the control conjugate. In additional embodiments, the analyte-binding conjugate and the control conjugate coat the surface of the liquid permeable membrane in the first portion. In other embodiments, the coating is absent from the sample application site. In additional embodiments, the test device further includes a fourth portion that acts as a wick, the fourth portion including sorbent material. In other embodiments, the second portion of the test device includes a liquid permeable material that acts as a filter to remove particulates. In still other embodiments, the first portion of the test device contains a conjugate that specifically binds platelets. In various embodiments, the conjugate that specifically binds platelets is one or more of an antibody to glycoprotein (GP) IIb, GP IIIa, GP V, GP Ib, GP IX, a lysosomal membrane protein, and platelet endothelial cell adhesion molecule (PECAM). In particular embodiments, the conjugate that specifically binds platelets is one or more of anti-CD41, anti-CD41a, anti-CD61, anti-CD42d, anti-CD42b, anti-CD42a, anti-CD63, and anti-CD31. In still other embodiments, the second portion of the test device includes an agent that alters the composition of the liquid as it contacts the second portion.

Compositions and methods for assaying platelet reactivity, as well as therapeutics working through FcγRIIa-binding are provided. Compositions and articles described herein were isolated or otherwise manufactured in accordance with the examples provided below. Other features and advantages of the disclosure will be apparent from the detailed description, and from the claims.

In one aspect of the present invention an isolated antibody or antigen binding fragment thereof that specifically binds to FcγRIIa is provided. The isolated antibody or antigen binding fragment thereof comprises at least one heavy chain complementarity-determining region (CDRH) and at least one light chain complementarity-determining region (CDRL). In some embodiments the CDRH is selected from SEQ ID NO: 5, SEQ ID NO: 6 and SEQ ID NO: 7 and the CDRL is selected from SEQ ID NO: 10, SEQ ID NO: 11 and SEQ ID NO: 12 wherein SEQ ID NO: 11 encodes the amino acid sequence DTS. In some embodiments the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22 wherein SEQ ID NO: 21 encodes the amino acid sequence WAS.

In some embodiments at least two heavy chain complementarity-determining regions (CDRH) and at least two light chain complementarity-determining regions (CDRL) are provided, wherein the CDRH is selected from SEQ ID NO: 5, SEQ ID NO: 6 and SEQ ID NO: 7 and the CDRL are selected from SEQ ID NO: 10, SEQ ID NO: 11 and SEQ ID NO: 12 or wherein the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22.

Some embodiments comprise an antibody or antibody binding fragment thereof comprising a CDRH1 region comprising SEQ ID NO: 5, a CDRH2 region comprising SEQ ID NO: 6, and a CDHR3 region comprising SEQ ID NO: 7; and a CDRL1 region comprising SEQ ID NO: 10, a CDRL2 region comprising SEQ ID NO: 11 and a CDRL3 region comprising SEQ ID NO: 12 and sequences at least 90% identical to SEQ ID NO: 5, SEQ ID NO: 6, SEQ ID NO: 7, SEQ ID NO: 10, SEQ ID NO: 11 and SEQ ID NO: 12. Some embodiments comprise an antibody or antibody binding fragment thereof comprising a CDRH1 region comprising SEQ ID NO: 15, a CDRH2 region comprising SEQ ID NO: 16, and a CDHR3 region comprising SEQ ID NO: 17; and a CDRL1 region comprising SEQ ID NO: 20, a CDRL2 region comprising SEQ ID NO: 21 and a CDRL3 region comprising SEQ ID NO: 22 and sequences at least 90% identical to SEQ ID NO: 15, SEQ ID NO: 16, SEQ ID NO: 17, SEQ ID NO: 10, SEQ ID NO: 11, SEQ ID NO: 12, SEQ ID NO: 20, SEQ ID NO: 21 and SEQ ID NO: 22.

In some embodiments, the antibody or antigen binding fragment thereof comprises a heavy chain complementarity-determining region encoded by SEQ ID NO: 4 and the light chain complementarity-determining region encoded by SEQ ID NO: 9 and sequences 90% identical to SEQ ID NO: 4 and SEQ ID NO: 9. In some embodiments, the antibody or antigen binding fragment thereof comprises the heavy chain complementarity-determining region is encoded by SEQ ID NO: 14 and the light chain complementarity-determining region is encoded by SEQ ID NO:19 and sequences 90% identical to SEQ ID NO: 14 and SEQ ID NO: 19.

In some embodiments, the antibody or antibody binding fragment thereof specifically binds to FcγRIIa on a platelet. In some embodiments, the antibody or antibody binding fragment thereof is linked to a detectable label. In some embodiments, the antibody is a monoclonal antibody. In some embodiments, the antibody or antibody binding fragment thereof is produced using recombinant antibody technology, nucleic acid aptamer technology or non-immunoglobulin protein scaffold technology. In some embodiments, the antibody or antibody binding fragment thereof specifically binds to FcγRIIa on a platelet that has been fix with a fixative before binding to the antibody or antibody binding fragment thereof.

In some embodiments, a method of detecting FcγRIIa comprising binding FcγRIIa to an antibody or antibody binding fragment thereof described herein is provided. In some embodiments a blood sample is treated with a fixative prior to or at the same time as the antibody or antibody binding fragment thereof described herein is introduced to the blood sample. In some embodiments the fixative is combined with the blood sample up to 2 days following collection of the blood sample. In some embodiments, the fixative is diluted in a solution comprising a buffer and glycerol prior to combining with a blood sample. In some embodiments, the fixative is diluted in a solution comprising a buffer and glycerol prior to combining with a blood sample. In some embodiments, the buffer is Phosphate Buffered Saline (PBS) and the glycerol comprises a range of approximately 2% to 7% of the total volume of the buffer-glycerol solution. In some embodiments, the glycerol is 5% of the total volume of the buffer-glycerol solution.

In some embodiments the bound FcγRIIa and antibody complex are detected via flow cytometry to measure the level of FcγRIIa in the sample. In some embodiments, the measured level of FcγRIIa in the sample is standardized based on a comparison of measured levels of fluorescence in samples with known levels of fluorescence markers.

A method detecting the presence of FcγRIIa in a blood sample comprising the steps of treating a blood sample with an anticoagulant, adding a fixative to the blood sample, separating and washing the platelets from the blood sample, incubating the platelets with an antibody, the antibody comprising any one of the antibodies or antigen binding fragments described herein and performing an analysis to quantify FcγRIIa in the sample is provided. In some embodiments, the analysis is selected from the group consisting of flow cytometry, immunoassay, ELISA, western blotting, and radioimmunoassay. In some embodiments, the method described herein further comprises the steps of reducing the concentration of the fixative in the blood sample and storing the blood sample from a period up to and including 1-14 days before incubating the platelets with the antibody.

In a second aspect the invention described herein provides a method of preparing a blood sample comprising the steps of, treating a blood sample with an anticoagulant, adding a fixative to the blood sample, subsequently diluting the concentration of the fixative in the blood sample, and storing the blood sample from a period up to and including 1-14 days. In some embodiments of the method, the fixative is added to a concentration of 5% and is subsequently diluted to 1.25%.

Another aspect of the invention provides a method of detecting FcγRIIa comprising binding FcγRIIa to an antibody, the antibody comprising at least one heavy chain complementarity-determining region (CDRH) and at least one light chain complementarity-determining region (CDRL). In some embodiments, the CDRH is selected from SEQ ID NO: 5, SEQ ID NO: 6 and SEQ ID NO: 7 and the CDRL are selected from SEQ ID NO: 10, SEQ ID NO: 11 and SEQ ID NO: 12 wherein SEQ ID NO: 11 encodes the amino acid sequence DTS. In some embodiments, the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22 wherein SEQ ID NO: 21 encodes the amino acid sequence WAS. In some embodiments FcγRIIa is detected on a platelet from a blood sample.

In some embodiments, the method further comprises comparing the level of detection of FcγRIIa between a blood sample of a human having, or suspected of having heart disease, thrombosis, coronary artery disease, renal disease, or myocardial infarction and a control human blood sample that does not have having heart disease, thrombosis, coronary artery disease, renal disease, or myocardial infarction.

In some embodiments a method of detecting platelet reactivity in a subject is provided. The method comprising detecting platelet reactivity with an antibody or antibody binding fragment thereof described herein.

### BRIEF DESCRIPTION OF THE DRAWINGS

FIGS. 1A-1E are western blots and graphs showing that platelet activation results in FcγRIIa being phosphorylated: Platelets (2×10⁸ in 0.5 ml) from low-risk subjects isolated with the use of gel filtration were exposed to no agonist (FIG. 1E) or to thrombin (50 nM) (FIG. 1A), convulxin (10 ng/ml) (FIG. 1B), ADP (25 μM) (FIG. 1C), and PAF (100 nM) (FIG. 1D) for selected intervals before preparation of platelet lysates. FcγRIIa was separated by immunoprecipitation and Western blots were probed with an antiphosphotyrosine antibody (4G10), stripped, and re-probed with anti-FcγRIIa to confirm equal loading (data not shown). Fluorescence intensity of bands was quantified with the use of a Li-Cor system. Results (n=3-5 for each condition) are peak intensity that is not altered by adjusting the contrast of the image or the size of the area of interest, and were compared with those obtained when no agonist was used (Student's t test, *p<0.05, **p<0.01). The inset shows a representative gel probed with the anti-phosphotyrosine antibody. In addition to the expected band at 45 kDa, bands at 55 kDa, 75 kDa, and 90 kDa showed evidence of phosphorylation. Activation with each agonist led to phosphorylation of FcγRIIa after 3 minutes. Results with thrombin and PAF were apparent earlier than those with ADP.

FIGS. 2A and 2B are a western blot and a gel. FIG. 2A is a western blot of an anti-phosphotyrosine immunoprecipitate showing that FcγRIIa is phosphorylated following platelet activation with thrombin. Tyrosine phosphorylated proteins (FIG. 2A) were isolated by immunoprecipitation with the use of an anti-phosphotyrosine antibody (4G10) from platelet lysates (2×10⁸ platelets) exposed to no agonist or thrombin (50 nM) for 3 min. Western blots of immunoprecipitated proteins in lanes 1 and 2 and a non-selected platelet lysate in lane 3 were probed with anti-FcγRIIa. Anti-FcγRIIa identified tyrosine phosphorylated proteins at 45 kDa, 55 kDa, 75 kDa, and 90 kDa. FIG. 2B is a representative gel of a lipid raft preparation showing results with non-activated (control) and activated (convulxin 10 ng/ml) platelets (4×10⁸ in 0.5 ml). Platelets were lysed after 1.5 min and lipid rafts were prepared (sucrose gradient). Lipid rafts were identified with the use of cholera toxin B (CTB). FcγRIIa was immunoprecipitated and Western blots were probed with an anti-phosphotyrosine antibody (4G10), stripped, and re-probed with anti-FcγRIIa. Bands were identified with the use of chemiluminescence.

FIG. 3 is a set of photomicrographs showing the subcellular localization of CD36 and FcγRIIa before and during platelet activation. Expression of FcγRIIa and CD36 by human platelets (top panel). PRP was prepared, and platelets were activated with thrombin (50 nM), convulxin (10 ng/ml), ADP (25 μM), and PAF (100 nM). After fixation with Optilyse (1.5% formaldehyde), platelets were pretreated with 1% BSA before incubation with a mouse anti-CD36 and a goat anti-FcγRIIa for 1 hr. After 3 washes in HT buffer, secondary antibodies were used to identify CD36 (Alexa 488 anti-mouse IgG) and FcγRIIa (Alexa 555 anti-goat IgG). Platelets were imaged with the use of a Zeiss LSM 510 META confocal/scanning laser microscope. For each condition, representative platelets on the right were imaged with differential interference contrast (phase contrast). Fluorescence imaging was used to identify CD36 (green signal) and FcγRIIa (red signal). Colocalization is identified by a yellow signal. Activation of platelets was associated with colocalization of lipid rafts and FcγRIIa. The effect of an antagonist of FcγRIIa (Fab fragment of IV.3) and an antagonist of GP IIb-IIIc (tirofiban) on the platelet expression of FcγRIIa (bottom panel). Non-activated platelets and thrombin-activated platelets were pretreated with IV.3 (100 μg/ml) or tirofiban (0.5 μg/ml). Representative platelets exhibited reduction in the clustering of FcγRIIa by pretreatment with IV.3. Magnification is 2,000×.

FIGS. 4A and 4B are graphs and western blots showing the expression of FcγRIIa (FIG. 4A) and the phosphorylation of FcγRIIa (FIG. 4B) in response to thrombin. Patients with previous myocardial infarction or coronary revascularization who were taking aspirin, but no other antiplatelet or anticoagulant agent, were screened to identify 3 patients who had elevated expression of FcγRIIa (mean fluorescence intensity 22). Platelet expression of FcγRIIa in low-risk subjects was found to be less than 1.5 units (n=5). To confirm results obtained with the use of flow cytometry, Western blot analysis was performed with platelet lysates (FIG. 4A insert). Platelets (2×10⁸ in 0.5 ml) isolated with the use of gel filtration were activated with thrombin (50 nM). Phosphorylation of FcγRIIa was quantified as described for FIGS. 1A-1E. Patients in whom expression of FcγRIIa was increased exhibited greater phosphorylation (p<0.05) after activation. Results are means±SD.

FIGS. 5A-5D are graphs showing the effect of a selective antagonist of FcγRIIa (Fab of IV.3) on the activation and aggregation of platelets. The activation of platelet was assessed with the use of flow cytometry by the binding of PAC-1 (reflecting activation of GP IIb-IIIa, FIG. 5A) or the surface expression of P-selectin (FIG. 5B). Whole blood from low-risk subjects (n=6) was added to reaction tubes containing fluorochrome labeled antibodies, selected agonists, and either control conditions or antagonist. Neither equimolar concentrations of non-immune IgG nor the Fab of non-immune IgG attenuated platelet activation (data not shown). IV.3 attenuated activation induced with each agonist, an effect that was most apparent when activation was identified by PAC-1 (FIG. 5A). The aggregation of platelets was assessed with light transmission aggregometry (FIG. 5C). Aggregation that was induced in platelet rich plasma (stirred and warmed to 37° C.) with collagen, thrombin receptor agonist peptide (TRAP), adenosine diphosphate (ADP), and platelet activating factor (PAF) (n=3 for each agonist) was inhibited by IV.3. FIG. 5D shows the effect of tirofiban (0.5 g/ml) on the activation of platelets (from n=3 subjects) identified by the surface expression of P-selectin. Tirofiban did not attenuate agonist-induced P-selectin expression, but consistent with its mechanism of action, tirofiban abolished binding of PAC-1 to platelets. Results are means±SD. Differences were identified with the use of paired Student's t test.

FIGS. 6A and 6B are graphs showing the effect of a non-selective antagonist of SRC kinase (PP2) on the activation of platelets. The activation of platelet was assessed with the use of flow cytometry by the binding of PAC-1 (FIG. 6A) or the surface expression of P-selectin (FIG. 6B). Whole blood from low-risk subjects (n=7) was added to reaction tubes containing fluorochrome labeled antibodies, selected agonists, and either control conditions (vehicle alone, dimethyl sulfoxide) or antagonist (PP2, 20 μM). Inhibition of tyrosine phosphorylation by PP2 inhibited activation induced by each agonist. Results are means±SD of the percentage inhibition (1-[PP2/control]). Differences between results with PP2 and control conditions were identified with the use of paired Student's t test.

FIG. 7 is a graph showing that the levels of FcγRIIa on the platelets from patients with end stage renal disease (ESRD) correlates with platelet reactivity.

FIGS. 8A and 8B are graphs showing levels of FcγRIIa on platelets of patients having coronary artery disease (CAD) or end stage renal disease (ESRD) relative to low-risk controls. FIG. 8A is a graph showing that platelets from patients with coronary artery disease (CAD) or end stage renal disease (ESRD) have higher levels of FcγRIIa relative to low-risk controls. FIG. 8B is a graph showing that platelets from patients with coronary artery disease (CAD) and 1 or more myocardial infarctions (MI) have higher levels of FcγRIIa relative to low-risk controls.

FIG. 9 is a table showing the list of inflammation associated cytokines and growth factors that effect FcγRIIa expression.

FIG. 10 shows that IFNγ increases the expression of FcγRIIa by megakaryocytes. The graph on the top demonstrates that IFNγ increases the expression of FcγRIIa by cells that exhibits characteristics of megakaryocytes. The graphs on the lower aspect of the figure show evidence of differentiation of the stem cells into megakaryocytes.

FIG. 11 shows that IFNγ increases the expression of FcγRIIa by human stem cell derived megakaryocytes. The graph on the top demonstrates that IFNγ increases the expression of FcγRIIa by a cell line that exhibits characteristics of megakaryocytes. The graphs on the lower aspect of the figure show evidence of differentiation of the cells into megakaryocytes.

FIG. 12 shows the effects of IFNγ on platelet expression of FcγRIIa.

FIG. 13 shows the effects of IFNγ on monocytic and myelocytic cell line expression of FcγRIIa.

FIG. 14 is a schematic diagram that illustrates the role of interrelation of FcγRIIa, platelet activation, and atherogenesis.

FIG. 15 is a graph showing that an FcγRIIa specific antibody activates platelets.

FIG. 16 provides a series of western blots showing FcγRIIa phosphorylation in thrombin activated platelets.

FIG. 17 shows the phosphorylation state of immunoprecipitated FcγRIIa from ADP activated platelets.

FIG. 18 is an illustration of lipid rafts.

FIG. 19 is an illustration showing the cytoskeletal rearrangement that happens during platelet activation.

FIG. 20 shows the effect of Coagulation Factor XIII on platelet activation.

FIG. 21 illustrates the role of FcγRIIa in platelet activation.

FIG. 22 is a table summarizing clinical events in patients with elevated platelet FcγRIIa expression.

FIG. 23 shows test results demonstrating the effect of time between blood draw and antibody incubation.

FIG. 24 shows test results where fixative was diluted before storage and later antibody incubation.

FIG. 25 shows test results with storage of blood samples from patients with elevated FcγRIIa expression.

FIG. 26 shows test demonstrating the effect of varying the time for fixation of the blood samples.

FIG. 27 shows test results demonstrating the effect of varying the dilution level of fixative following 15 minutes of fixation with 0.5% formaldehyde.

FIG. 28 shows test results demonstrating the effect of varying the initial concentration of fixative on blood samples that were fixed for 15 minutes with formaldehyde, within 2 hours of sample collection.

FIG. 29 shows test results for two different subjects comparing a commercially available FcγRIIa antibody with the 5G1 antibody described herein.

FIG. 30 shows that platelets can be fixed up to 2 days after blood is drawn from a subject.

FIG. 31 shows sample stability over time after fixation.

FIG. 32 shows sample stability over time after fixation PBS-glycerol diluted fixation buffer.

### DETAILED DESCRIPTION OF THE DISCLOSURE

The disclosure features compositions and methods that are useful for determining platelet reactivity in a biological sample of a subject, identifying subjects that are at increased risk of thrombosis, and selecting appropriate therapies for such high risk subjects, including subjects at high risk for subsequent cardiovascular events.

The disclosure is based, at least in part, on the discovery that FcγRIIa contributes to (e.g., amplifies) the activation of platelets and thus greater expression of FcγRIIa increases the extent of activation of platelets; that FcγRIIa protein levels per platelet correlate with disease state (e.g., levels of FcγRIIa protein/platelet are two-five fold increased in subjects with atherosclerosis and diabetes, and two-ten fold increased in subjects with end stage renal disease); and that FcγRIIa levels are useful in identifying subjects having an increased propensity to develop thrombosis and related thrombotic disease and in selecting appropriate therapies for such subjects. In particular, an increase in platelet reactivity is useful in identifying a subject that could benefit from more aggressive drug treatments (e.g., treatment with a more powerful anti-platelet agent, such as Clopidogrel, Prasugrel, Ticagrelor or Vorapaxar).

Accordingly, the disclosure provides methods for measuring FcγRIIa because it increases platelet reactivity and as such is a marker of elevated platelet reactivity, including flow cytometry and immunoassay-based methods, diagnostic methods employing FcγRIIa as a marker of platelet reactivity, and methods for selecting an appropriate therapeutic agent for a subject identified as having increased platelet reactivity relative to a reference. Advantageously, the diagnostic methods of the disclosure can be used on a biological sample (e.g., blood, serum, and plasma) obtained from a subject being treated with an antiplatelet or anticoagulant agent.

In certain embodiments, the disclosure provides a test device, such as a lateral flow device, that comprises a liquid permeable media that provides for the flow of a liquid sample (e.g., blood, serum, plasma) through the device. Test devices of the disclosure can be used for the detection of an analyte of interest (FcγRIIa) by a detectably labeled reactant capable of specifically interacting with the analyte (FcγRIIa). The test device described herein is particularly suitable for the detection of an antigen of interest using an antibody that specifically binds the antigen and conventional immunoassay procedures.

### FcγRIIa

As reported in more detail below, it was found that when platelet activation induces cytoskeletal rearrangement that FcγRIIa clusters in cytoskeletal lipid rafts. The clustering leads to phosphorylation when FcγRIIa is cross-linked with fibrinogen and coagulation Factor XIII. Phosphorylation of FcγRIIa leads to downstream phosphorylation and ultimately the release of calcium that augments the activation of platelets. Consistent with the association of FcγRIIa with membrane cytoskeletal proteins during activation, results with confocal microscopy and preparations of lipid rafts demonstrated clustering of FcγRIIa confined to membrane cytoskeletal lipid rafts. The results presented herein indicate that rearrangement of membrane cytoskeletal proteins during activation is associated with clustering of FcγRIIa that appears to promote its cross-linking by fibrinogen and Factor XIII. Cross-linking by fibrinogen and Factor XIII leads to phosphorylation by SRC kinases (e.g. Lyn).

Further, fibrinogen and Factor XIII co-immunoprecipitated with FcγRIIa from activated platelets and increased activation of platelets. Inhibition of the binding of fibrinogen to GP IIb-IIIa did not abolish amplification of activation by fibrinogen. Further, platelet activation induced by an activating anti-FcγRIIa antibody was not attenuated by tirofiban. These results indicate that interaction between FcγRIIa and GP IIb-IIIa is sufficient but perhaps not necessary for FcγRIIa to contribute to platelet activation.

Amplification of platelet activation induced by fibrinogen was abolished by IV.3 Fab, an antibody that is a specific inhibitor of FcγRIIa, but not by tirofiban. In contrast, the activation of platelets caused by coagulation Factor XIII was abolished by both IV.3 and tirofiban. Without being bound by any particular theory, this finding is consistent with the hypothesis that the cross-linking of FcγRIIa homodimers with fibrinogen or an anti-FcγRIIa antibody and the cross-linking of heterodimers (FcγRIIa and GP IIb-IIIa) by coagulation Factor XIII leads to phosphorylation of FcγRIIa that amplifies the activation of platelets.

Inhibition of phosphorylation of FcγRIIa appeared to have less effect with higher concentrations of thrombin. This observation is consistent with previous results (Canobbio I, et al., Cell Signal 2006; 18:861-70) and indicates that phosphorylation of FcγRIIa is not necessary for activation of platelets. Phosphorylation of FcγRIIa appears to amplify the activation of platelets much in the same way that the release of thromboxane A2 and ADP during the process of activation amplifies the extent of platelet activation (Murray R, et al., Proc Natl Acad Sci USA 1989; 86:124-8; and Storey R F, et al., Platelets 2001; 12:443-7). These results are consistent with greater platelet reactivity that has been observed when platelet expression of FcγRIIa is increased (Calverley D C, et al., Atherosclerosis 2002; 164:261-7; Canobbio I, et al., Cell Signal 2006; 18:861-70; and Serrano F A, et al., Thromb J 2007; 5:7).

Coagulation Factor XIII has a powerful effect on the activation of platelets, increasing the extent of activation by nearly 4-fold. These results indicate that the effect of Factor XIII is mediated by FcγRIIa. Furthermore, inhibition of SRC kinase (downstream kinases) by PP2 attenuated activation of platelets. In view of the essential role of Fcγ in GP VI mediated activation, the effect was most profound with convulxin-induced activation.

By “FcγRIIa” is meant the low affinity immunoglobulin gamma Fc region receptor II-a. An illustrative amino acid sequence of the FcγRIIa is provided at GenBank Accession No. NP_001129691.1: (SEQ ID NO 1)

| | |
|---|---|
| 1 | mtmetqmsqn vcprnlwllq pltvllllas adsqaaappk |
| | avlkleppwi nvlqedsvtl |
| 61 | tcqgarspes dsiqwfhngn lipthtgpsy rfkannndsg |
| | eytcqtgqts lsdpvhltvl |
| 121 | sewlvlqtph lefqegetim lrchswkdkp lvkvtffqng |
| | ksqkfshldp tfsipqanhs |
| 181 | hsgdyhctgn igytlfsskp vtitvqvpsm gssspmgiiv |
| | avviatavaa ivaavvaliy |
| 241 | crkkrisans tdpvkaaqfe ppgrqmiair krqleetnnd |
| | yetadggymt lnpraptddd |
| 301 | kniyltlppn dhvnsnn. |

An illustrative nucleic acid sequence encoding FcγRIIa is provided at GenBank Accession No.

ATGACTATGGAGACCCAAATGTCTCAGAATGTATGTCCCAGAAACCTGTG
GCTGCTTCAACCATTGACAGTTTTGCTGCTGCTGGCTTCTGCAGACAGTC
AAGCTGCAGCTCCCCCAAAGGCTGTGCTGAAACTTGAGCCCCCGTGGATC
AACGTGCTCCAGGAGGACTCTGTGACTCTGACATGCCAGGGGGCTCGCAG
CCCTGAGAGCGACTCCATTCAGTGGTTCCACAATGGGAATCTCATTCCCA
CCCACACGCAGCCCAGCTACAGGTTCAAGGCCAACAACAATGACAGCGGG
GAGTACACGTGCCAGACTGGCCAGACCAGCCTCAGCGACCCTGTGCATCT
GACTGTGCTTTCCGAATGGCTGGTGCTCCAGACCCCTCACCTGGAGTTCC
AGGAGGGAGAAACCATCATGCTGAGGTGCCACAGCTGGAAGGACAAGCCT
CTGGTCAAGGTCACATTCTTCCAGAATGGAAAATCCCAGAAATTCTCCCA
TTTGGATCCCACCTTCTCCATCCCACAAGCAAACCACAGTCACAGTGGTG
ATTACCACTGCACAGGAAACATAGGCTACACGCTGTTCTCATCCAAGCCT
GTGACCATCACTGTCCAAGTGCCCAGCATGGGCAGCTCTTCACCAATGGG
GATCATTGTGGCTGTGGTCATTGCGACTGCTGTAGCAGCCATTGTTGCTG
CTGTAGTGGCCTTGATCTACTGCAGGAAAAAGCGGATTTCAGCCAATTCC
ACTGATCCTGTGAAGGCTGCCCAATTTGAGCCACCTGGACGTCAAATGAT
TGCCATCAGAAAGAGACAACTTGAAGAAACCAACAATGACTATGAAACAG
CTGACGGCGGCTACATGACTCTGAACCCCAGGGCACCTACTGACGATGAT
AAAAACATCTACCTGACTCTTCCTCCCAACGACCATGTCAACAGTAATAA
CTAA.

By “FcγRIIa specific agent” is meant any small molecule compound, antibody, nucleic acid molecule, or polypeptide, or fragments thereof that specifically bind to FcγRIIa.

By “platelet reactivity” is meant the sensitivity of platelets to activation and clotting. Platelet reactivity testing is based on the distinction between low platelet reactivity and high platelet reactivity. Platelet activity or reactivity includes but is not limited to platelet adhesion and aggregation. Platelet reactivity is a determinant of thrombosis.

A clinical phenotype of increased platelet reactivity and evidence of an increased risk of thrombosis was identified when platelet expression of FcγRIIa is increased. Greater platelet expression of FcγRIIa was observed in blood from patients with previous stroke, myocardial infarction, and unstable angina. Somewhat more compelling evidence of a thrombotic phenotype is provided by the association of a greater risk of subsequent thrombotic events in patients with end stage renal disease who have greater platelet expression of FcγRIIa (EI-Shahawy M, et. al., Am. J Kidney Dis. 2007; 49:127-34). Among patients with myocardial infarction (MI), high platelet expression of FcγRIIa (≥11,000 copies/platelet) was associated with a greater risk (odds ratio, >4) of MI, stroke, and death. Platelet expression of FcγRIIa can be used to identify patients at high and low risk of events after an MI or for patients with CAD.

The results disclosed herein demonstrate that activated platelets have phosphorylated FcγRIIa associated with membrane cytoskeletal proteins, fibrinogen, and coagulation Factor XIII. These results indicate that the activation of platelets leads to plasma membrane cytoskeletal rearrangement, the clustering of FcγRIIa, and the cross-linking of FcγRIIa by fibrinogen and Factor XIII in association with lipid raft proteins. Lipid raft proteins (SRC kinases) phosphorylate FcγRIIa that leads to downstream signaling and serves to amplify the activation of platelets.

Based on the results reported herein, it was discovered that increased levels of FcγRIIa on platelets causes and is therefore an indicator of increased platelet reactivity. Moreover, the use of FcγRIIa is superior to other measures of platelet reactivity, the level of FcγRIIa is not influenced by commonly used therapies and medicines. Thus, the use of platelet FcγRIIa as a marker of platelet reactivity is not influenced by antiplatelet treatment.

### Diagnostics

The present disclosure features diagnostic assays for the identification of subjects having an increased level of FcγRIIa, which is indicative of high platelet reactivity, and an increased risk of thrombotic disease. In one embodiment, levels of platelet FcγRIIa are measured in a subject sample and used to characterize platelet reactivity in the subject. Any suitable method can be used to detect platelet FcγRIIa in a subject sample and used to characterize platelet reactivity in blood from the subject. Biological samples include bodily fluids (e.g., blood, blood serum, plasma, and saliva). Successful practice of the disclosure can be achieved with one or a combination of methods that can detect and/or quantify platelet FcγRIIa. Immunoassays in various formats (e.g., flow cytometry, ELISA) are popular methods for detection of analytes captured on a solid phase. Such methods typically involve use of an FcγRIIa-specific antibody.

Virtually any method known in the art can be used to detect FcγRIIa. For example, levels of platelet FcγRIIa are compared by procedures well known in the art, such as flow cytometry, immunoassay, ELISA, western blotting, radioimmunoassay, immunocytochemistry, binding to magnetic and/or antibody-coated beads, in situ hybridization, fluorescence in situ hybridization (FISH), flow chamber adhesion assay, microarray analysis, or colorimetric assays. Methods may further include, one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero.

Detection methods may include use of a biochip array. Biochip arrays useful in the disclosure include protein and polynucleotide arrays. One or more markers are captured on the biochip array and subjected to analysis to detect the level of the markers in a sample.

Platelet FcγRIIa may be captured with capture reagents fixed to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltammetry and amperometry methods. Radio frequency methods include multipolar resonance spectroscopy.

Mass spectrometry (MS) is a well-known tool for analyzing chemical compounds. Thus, in one embodiment, the methods of the present disclosure comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). The preferred procedure is MALDI with time of flight (TOF) analysis, known as MALDI-TOF MS. This involves forming a matrix on the membrane, e.g. as described in the literature, with an agent which absorbs the incident light strongly at the particular wavelength employed. The sample is excited by UV, or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Frazingham, Mass., USA) and are described in the literature, e.g. M. Kussmann and P. Roepstorff, cited above.

In other embodiments, levels of FcγRIIa are detected in combination with one or more additional markers. While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, methods of the present disclosure provide for the measurement of more than one marker or clinical parameter.

The use of multiple markers increases the predictive value of the test and provides greater utility in diagnosis, toxicology, patient stratification and patient monitoring. The process called “Pattern recognition” detects the patterns formed by multiple markers. The inclusion of additional markers may improve the sensitivity and specificity in determining a patient's risk for developing a thrombotic disease or disorder associated with an undesirable increase in platelet reactivity. Subtle variations in data from clinical samples indicate that certain patterns of protein level or expression (e.g., FcγRIIa level) can predict phenotypes such as an increase in platelet reactivity, or can identify a patient that could benefit from more aggressive drug treatments (e.g., treatment with a more powerful anti-platelet agent, such as Clopidogrel, Prasugrel, Ticagrelor, or Vorapaxar).

Expression levels of platelet FcγRIIa are correlated with platelet reactivity, and thus are useful in diagnosis. Antibodies that specifically bind FcγRIIa, or any other method known in the art may be used to monitor expression of platelet FcγRIIa. Detection of an alteration relative to a normal, reference sample can be used as a diagnostic indicator of platelet reactivity. In particular embodiments, a 1.3-2, 3, 4, 5, or 6-fold difference in the level of platelet FcγRIIa or MFI measurements is indicative of platelet reactivity.

In one embodiment, the level of platelet FcγRIIa is measured on at least two different occasions and an alteration in the levels as compared to normal reference levels over time is used as an indicator of platelet reactivity or the propensity to develop thrombosis. In general, levels of platelet FcγRIIa are present at low levels (about 1,500 copies per platelet) in a low-risk subject (e.g., those who do not have reactive platelets). In one embodiment an increased level of platelet FcγRIIa (of about 1,600, 1,700, 1,800, 1,900, 2,000, 2,100, 2,200, 2,300, 2,400 or 2,500 copies of FcγRIIa per platelet, or more, is indicative of platelet reactivity. The increased level may be any level at or above 1,600, 1,700, 1,800, 1,900, 2,000, 2,100, 2,200, 2,300, 2,400 or 2,500 copies of FcγRIIa per platelet, or more, including ranges of from 1,600-2500, 1,700-2,400, 1,800-2,300, 1,900-2,200 as well as all of the various combinations of these ranges and the points included therein.) Preferably, FcγRIIa copy/platelet is measured using FACS analysis.

In one embodiment the reference value is at or about 1,500 copies of FcγRIIa per platelet and the increased level is at or about 1,600, 1,700, 1,800, 1,900, 2,000, 2,100, 2,200, 2,300, 2,400 or 2,500 copies of FcγRIIa per platelet, or more. The increased level may be any level at or above 1,600, 1,700, 1,800, 1,900, 2,000, 2,100, 2,200, 2,300, 2,400 or 2,500 copies of FcγRIIa per platelet, or more.

The diagnostic methods described herein can be used individually or in combination with any other diagnostic method described herein for a more accurate diagnosis of the presence or severity of thrombotic disease.

The correlation may take into account the amount of platelet FcγRIIa in the sample compared to a control amount of platelet FcγRIIa (e.g., in low-risk subjects or in subjects where platelet reactivity is undetected). A control can be, e.g., the average or median amount of platelet FcγRIIa present in comparable samples of normal subjects. The control amount is measured under the same or substantially similar experimental conditions as in measuring the test amount. As a result, the control can be employed as a reference standard, where the normal phenotype is known, and each result can be compared to that standard, rather than re-running a control.

Accordingly, a marker profile may be obtained from a subject sample and compared to a reference value obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate determination of cancer status.

In certain embodiments, the methods further comprise selecting anti-thrombotic therapy. For example, where a 1.3-5 fold, 5-10 fold, or 10-25 fold increase in platelet reactivity relative to a reference as indicated by measurement of FcγRIIa MFI or actual copy numbers identifies a patient that could benefit from more aggressive drug treatments (e.g., treatment with a more powerful anti-platelet agent, such as Clopidogrel, Prasugrel, Ticagrelor, or Vorapaxar). The disclosure also provides for such methods where platelet FcγRIIa is measured again after anti-thrombotic therapy. In these cases, the methods are used to monitor the status of the platelet reactivity.

### Antibodies

Antibodies are glycoproteins. The basic functional unit of each antibody is an immunoglobulin (Ig) monomer (containing only one Ig unit). The Ig monomer is a “Y”-shaped molecule that consists of four polypeptide chains; two identical heavy chains and two identical light chains connected by disulfide bonds. Each chain is composed of structural domains called immunoglobulin domains. The immunoglobulin domains are composed of between 7 (for constant domains) and 9 (for variable domains). The type of heavy chain present defines the class of antibody; these chains are found in IgA, IgD, IgE, IgG, and IgM antibodies, respectively. Each heavy chain has two regions, the constant region and the variable region. The constant region is identical in all antibodies of the same isotype but differs in antibodies of different isotypes. The variable region of the heavy chain differs in antibodies produced by different B cells but is the same for all antibodies produced by a single B cell or B cell clone. In mammals there are two types of immunoglobulin light chain, which are called lambda and kappa. A light chain has two successive domains: one constant domain and one variable domain.

The variable domain is also referred to as the Fv region and is the most important region for binding to antigens. More specifically, variable loops (three each on the light (VL) and heavy (VH) chains) are responsible for binding to the antigen. These loops are referred to as the complementarity determining regions (CDRs).

By “antigen-binding fragment thereof” or “fragment thereof”, we refer to a portion of the polypeptide that retains its ability to specifically and selectively bind to the antigen or target. In this application, the antigen or target is FcγRIIa. Suitably, the antigen-binding fragment thereof will contain the antigen-binding regions of the VLR in order to maintain its ability to selectively and specifically bind to FcγRIIa. One skilled in the art, using the methods described in the examples below, will readily be able to determine suitable antigen-binding fragments which are able to bind to FcγRIIa.

As reported herein, antibodies that specifically bind FcγRIIa are useful in diagnostic, as well as therapeutic methods. For example, antibodies that act as platelet FcγRIIa antagonists (e.g., IV.3 Fab) are particularly useful in the methods of the disclosure. In particular embodiments, the disclosure provides methods of using anti-platelet FcγRIIa antibodies for the inhibition of platelet reactivity. IV.3 is a monoclonal anti-FcγRIIa antibody that inhibits the phosphorylation of platelet FcγRIIa during platelet activation.

Other antibodies useful in the disclosure are those that attenuate platelet FcγRIIa signaling. Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments that lack the Fc fragment of an intact antibody, clear more rapidly from the circulation, and may have less non-specific tissue binding than an intact antibody (Wahl et al., J. Nucl. Med. 24:316-325 (1983). The antibodies of the disclosure comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062,1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991).

Single chain Fv polypeptide antibodies include a covalently linked VH::VL heterodimer which can be expressed from a nucleic acid including VH- and VL-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, an antibody that binds platelet FcγRIIa is monoclonal. Alternatively, the anti-platelet FcγRIIa antibody is a polyclonal antibody. The preparation and use of polyclonal antibodies are also known to the skilled artisan. The disclosure also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)2” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing soluble polypeptides, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding human FcγRIIa or immunogenic fragments thereof, can be provided to the host in a delivery vehicle that is taken up by immune cells of the host. The cells will in turn express the human FcγRIIa thereby generating an immunogenic response in the host. Alternatively, nucleic acid sequences encoding human FcγRIIa or immunogenic fragments thereof, can be expressed in cells in vitro, followed by isolation of the human FcγRIIa and administration of the FcγRIIa to a suitable host in which antibodies are raised.

Alternatively, antibodies against platelet FcγRIIa may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface. Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the disclosure can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Antibody FcYRIIa-5G1

In one embodiment an antibody referred to as FcYRIIa-5G1 is provided. The heavy chain of the antibody FcYRIIa-5G1 is encoded by the following DNA sequence: (SEQ ID NO 3)

gaggtgcagctgcaggagtctggacctgagctggtgaagcctggggcttc
agtgaagatatcctgcaaggcttctggctacaccttcacaacctactata
tacactgggtgaagcagaggcctggacagggacttgagtggattggatgg
atttttcctggaagtagtaatactttctacaatgagaaattcaagggcaa
ggccacactgacggcagacacatccgccatcactgcctacatgcagctca
acagcctgacatctgaagactctgcagtctatttctgttcaagatattac
tacgactatggttactggggccaagggactctggtcactgtctctgcag

The predicted protein sequence based on this DNA sequence encoding the heavy chain of antibody FcYRIIa-5G1 is provided below, with the complementarity determining regions (CDRs) underlined. (SEQ ID NO 4)

EVQLQESGPELVKPGASVKISCKASGYTFTTYYIHWVKQRPGQGLEWIGW
IFPGSSNTFYNEKFKGKATLTADTSAITAYMQLNSLTSEDSAVYFCSRYY
YDYGYWGQGTLVTVSA

In particular, the CDRs for the heavy chain of antibody FcYRIIa-5G1 are:

(SEQ ID NO 5)
CDRH1: GYTFTTYY
(SEQ ID NO 6)
CDRH2: IFPGSSNT
(SEQ ID NO 7)
CDRH3: SRYYYDYGY

The light chain of the antibody FcYRIIa-5G1 is encoded by the following DNA sequence: (SEQ ID NO 8)

gaaaatgttctcacccagtctccagcaatcatgtctgcatctccagggga
aaaggtcaccatgacctgcagtgccagctcaagtgttcgttacatgcaat
ggtaccagcagaagtcaaacacctcccccaaactctggatttatgacaca
tccaaactggcttctggagtcccaggtcgcttcagtggcagtgggtctgg
aaactcttactttctcacgatcagcagcatggaggctgaagatgttgcca
cttatcactgttttcaggggagtggttacccactcacgttcggagggggg
accaagctggaaataaaac

The predicted protein sequence based on this DNA sequence encoding the light chain of antibody FcYRIIa-5G1 is provided below, with the complementarity determining regions (CDRs) underlined. (SEQ ID NO 9)

ENVLTQSPAIMSASPGEKVTMTCSASSSVRYMQWYQQKSNTSPKLWIYDT
SKLASGVPGRFSGSGSGNSYFLTISSMEAEDVATYHCFQGSGYPLTFGGG
TKLEIK

In particular, the CDRs for the light chain of antibody FcYRIIa-5G1 are:

(SEQ ID NO 10)
CDRL1: SSVRY
(SEQ ID NO 11)
CDRL2: DTS
(SEQ ID NO 12)
CDRL3: FQGSGYPLT

### Antibody FcYRIIa-11F9

In another embodiment an antibody referred to as FcYRIIa-11F9 is provided. The heavy chain of the antibody FcYRIIa-11F9 is encoded by the following DNA sequence: (SEQ ID NO 13)

gaggtgcagctgcaggagtctggacctgagctggtgaagcctggggcttc
agtgaggatttcctgcaaggcctctgggtacaccttcacaaactcctata
tgcactgggtgaagcagaggcctggacagggacttgagtggattggatgg
atttatcctgaaaatattaatacttattacaatgagaagttcaagggcaa
ggccacactgactgcagacagatcctccagcacagcctacatgcagctca
acagcctggcctctgaggactctgcggtctatttctgtgcaagattttat
tacggcttcggctactggggccaaggcaccactctcacagtctcctcag

The predicted protein sequence based on this DNA sequence encoding the heavy chain of antibody FcYRIIa-11F9 is provided below, with the complementarity determining regions (CDRs) underlined. (SEQ ID NO 14)

EVQLQESGPELVKPGASVRISCKASGYTFTNSYMHWVKQRPGQGLEWIGW
IYPENINTYYNEKFKGKATLTADRSSSTAYMQLNSLASEDSAVYFCARFY
YGFGYWGQGTTLTVSS

In particular, the CDRs for the heavy chain of antibody FcYRIIa-11F9 are:

(SEQ ID NO 15)
CDRH1: GYTFTNSY
(SEQ ID NO 16)
CDRH2: IYPENINT
(SEQ ID NO 17)
CDRH3: ARFYYGFGY

The light chain of the antibody FcYRIIa-11F9 is encoded by the following DNA sequence: (SEQ ID NO 18)

gacattgtgatgacccagtctcacaagttcatgtccacatcagtgggaga
cagggtcatcatcacctgcaaggccagtcagaatgtgatttctgctgtag
cctggtatcaacagaaaccaggacattctcctaaactactgatttattgg
gcatccacccggcacactggagtccctgatcgcttcacaggcagtggatc
tgggacagatttcactctcaccattaacagtgtgcagtctgaggacttgg
cagattatttctgtcagcaatatggcaggtatccttacacgttcgcaggg
gggaccaagctggaaataaaac

The predicted protein sequence based on this DNA sequence encoding the light chain of antibody FcYRIIa-11F9 is provided below, with the complementarity determining regions (CDRs) underlined. (SEQ ID NO 19)

DIVMTQSHKFMSTSVGDRVIITCKASQNVISAVAWYQQKPGHSPKLLIYW
ASTRHTGVPDRFTGSGSGTDFTLTINSVQSEDLADYFCQQYGRYPYTFAG
GTKLEIK

In particular, the CDRs for the light chain of antibody FcYRIIa-11F9 are:

(SEQ ID NO 20)
CDRL1: QNVISA
(SEQ ID NO 21)
CDRL2: WAS
(SEQ ID NO 22)
CDRL3: QQYGRYPYT

The disclosed monoclonal antibodies FcYRIIa-5G1 and FcYRIIa-11F9 can bind to FcγRIIa or certain subregions thereof, with high affinity, specificity, and selectivity.

Some embodiments comprise a FcYRIIa-5G1 antibody comprising CDRH1 (SEQ ID NO: 5), CDRH2 (SEQ ID NO: 6), CDRH3 (SEQ ID NO: 7), CDRL1 (SEQ ID NO: 10), CDRL2 (SEQ ID NO: 11), CDRL3 (SEQ ID NO: 12). Some embodiments comprise a FcYRIIa-5G1 antibody comprising VH (SEQ ID NO: 4) and VL (SEQ ID NO: 9) or SEQ ID NO: 3 and SEQ ID NO: 8.

Some embodiments comprise a FcYRIIa-11F9 antibody comprising CDRH1 (SEQ ID NO: 15), CDRH2 (SEQ ID NO: 16), CDRH3 (SEQ ID NO: 17), CDRL1 (SEQ ID NO: 20), CDRL2 (SEQ ID NO: 21), CDRL3 (SEQ ID NO: 22). Some embodiments comprise a FcYRIIa-11F9 antibody comprising VH (SEQ ID NO: 14) and VL (SEQ ID NO: 19) or SEQ ID NO: 13 and SEQ ID NO: 18.

Sequences of the antibodies FcYRIIa-5G1 and FcYRIIa-11F9 were determined as follows. Total RNA was isolated from the hybridoma cell line culture (2×10⁶ cells in each case). RNA was treated to remove aberrant transcripts and reverse transcribed using oligo(dT) primers. Samples of the resulting cDNA were amplified in separate PCRs using framework 1 and constant region primer pairs specific for either the heavy or light chain. Reaction products were separated on an agarose gel, and size-evaluated. PCR reactions were prepared for sequencing using a PCR clean up kit and sequenced at GENEWIZ using an Illumina® NovSeq 6000.

DNA sequence data from all constructs was analyzed and consensus sequences for heavy and light chain were determined. The consensus sequences were compared to all known variable region sequences to rule out artifacts and/or process contamination. Consensus sequences were then analyzed using an online tool to verify that the sequences could encode a productive immunoglobulin.

### Preparation of Antibodies

Purified FcγRIIa (produced by mammalian cells) was exposed to formaldehyde before injection into mice. Mouse serum and cell culture media from hybridomas were screened for antibodies that bound to FcγRIIa that had been exposed to formaldehyde. A direct immunoassay with the formaldehyde treated FcγRIIa was used to screen for the presence of antibodies of interest. To identify clones producing antibodies with high binding affinity, competition immunoassays were performed. Clones whose antibodies competed effectively with a commercial antibody were selected for further evaluation. For these immunoassays, FcγRIIa used had not been exposed to formaldehyde. This approach was used to identify clones that not only bound FcγRIIa on “fixed” platelets but did so with high affinity, demonstrating that the sensitivity of the antibodies developed is suitably high.

Candidate clones were evaluated with a series of additional tests. Chinese hamster ovary (CHO) cells were stably transfected with FcγRIIa to assess binding of the clones with the use of fluorescence microscopy and flow cytometry. In addition, antibodies from candidate clones were used to perform immunoprecipitation with platelet lysates. These experiments confirmed that binding was specific to FcγRIIa (no other proteins were immunoprecipitated). Based on these screening tests, high binding candidate clones were selected.

One objective was to identify an antibody that was capable of quantifying FcγRIIa on the surface of platelets that were fixed, similar to quantification of FcγRIIa on non-fixed platelets. To make this assessment, selected antibodies were fluorochrome labeled. An objective was to identify a clone that could quantify FcγRIIa on the surface of fixed platelets within 10% of the quantification of previously-known antibodies on non-fixed platelets. Based on these experiments, two clones were identified, they produced the antibodies FcYRIIa-5G1 and FcYRIIa-11F9 described above.

Antibodies from the selected FcγRIIa clones were tested for sensitivity and specificity. Each antibody was selected for not only its ability to bind FcγRIIa that had been exposed to fixative but also for its ability to competitively inhibit binding of a commercial FcγRIIa antibody to non-fixed FcγRIIa. Specificity was assessed by performing immunoprecipitation experiments with platelet lysates. In these experiments, FcγRIIa was the only protein precipitated.

Antibodies may also be obtained in conventional ways including steps of introducing FcγRIIa, subregions, peptides, or degenerate versions thereof, into a host animal, followed by isolation of the antibody-producing spleen cells and formation of a suitable hybridoma.

Also contemplated herein are antibodies such as, and including, those described above, that have been bound to a detectable label. In a preferred embodiment the label is bound to the antibody in a 1:1 ratio to allow for comparison of detection output measurements and comparison against a curve comparing instrument output (for instance fluorescence measurements) to known levels of detectable label, as described below.

### Inhibitory Nucleic Acids

Inhibitory nucleic acid molecules are those oligonucleotides that inhibit the expression or activity of platelet FcγRIIa for the prevention of thrombosis and the treatment of thrombosis-related disorders. Such oligonucleotides include single and double stranded nucleic acid molecules (e.g., DNA, RNA, and analogs thereof) that bind a nucleic acid molecule that encodes FcγRIIa (e.g., antisense molecules, siRNA, shRNA) as well as nucleic acid molecules that bind directly to a platelet FcγRIIa polypeptide to modulate its biological activity (e.g., aptamers).

### Ribozymes

Catalytic RNA molecules or ribozymes that target an antisense FcγRIIa sequence of the present disclosure can be used to inhibit expression of a FcγRIIa nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. Accordingly, the disclosure also features a catalytic RNA molecule that includes, in the binding arm, an antisense RNA having between eight and nineteen consecutive nucleobases. In preferred embodiments of this disclosure, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the disclosure and those skilled in the art will recognize that all that is important in an enzymatic nucleic acid molecule of this disclosure is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002). Given the sequence of a target gene, siRNAs may be designed to inactivate that gene. Such siRNAs, for example, could be administered directly to an affected tissue, or administered systemically. The nucleic acid sequence of an Parl gene can be used to design small interfering RNAs (siRNAs). The 21 to 25 nucleotide siRNAs may be used, for example, as therapeutics to treat lupus.

The inhibitory nucleic acid molecules of the present disclosure may be employed as double-stranded RNAs for RNA interference (RNAi)-mediated knock-down of platelet FcγRIIa expression. In one embodiment, platelet FcγRIIa expression is reduced in megakaryocytes. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the disclosure, a double-stranded RNA (dsRNA) molecule is made that includes between eight and nineteen consecutive nucleobases of a nucleobase oligomer of the disclosure. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Therapeutic Applications

Also disclosed is a method of treating an animal or human by administering an effective amount of an antibody against FcγRIIa. The antibody may include or be provided along with or conjoined to a platelet specific antibody, for instance in the form of a bi-specific antibody. Such antibodies are also considered within the scope of this disclosure. These antibodies may include FcYRIIa-5G1 and FcYRIIa-11F9 disclosed herein or portions thereof, for instance one or more of the CDRs. Also contemplated herein are antibodies which are 80%, 85%, 90%, 95%, 98% homologous to all or a portion of FcYRIIa-5G1 and FcYRIIa-11F9 disclosed herein.

Also disclosed herein are molecules which are or include an antigen binding fragment and/or derivative of FcYRIIa-5G1 and FcYRIIa-11F9 disclosed herein, which comprises any one or more of the CDRs disclosed herein, including CDRs from both of FcYRIIa-5G1 and FcYRIIa-11F9 disclosed herein in a single molecule.

Also disclosed are antigen binding proteins, such as an antibody or antigen binding fragment thereof which specifically binds FcYRIIa and comprises CDR's which are variants of the sequences set forth in CDR SEQ ID Nos 5, 6, 7, 10, 11, 12, 15, 16, 17, 20, 21, 22.

A variant includes a partial alteration of one or more CDRs, heavy chain and/or light chain amino acid sequence by deletion or substitution of one to several amino acids of the CDRs, heavy chain and/or light chain, and/or by addition or insertion of one to several amino acids to the CDR, heavy chain, and/or light chain, and/or by a combination thereof. The variant may contain 1, 2, 3, 4, 5, or 6 amino acid substitutions, additions and/or deletions (1-6 each) in the amino acid sequence of the CDRs, heavy chain, and/or light chain sequence. The substitutions in amino acid residues may be conservative substitutions, for example, substituting one hydrophobic amino acid for an alternative hydrophobic amino acid.

Antigen binding proteins which are variants of one or more CDRs, heavy chain, and/or light chain will have the same or similar functional properties to those comprising the CDR, heavy chain, and/or light chain described herein. Therefore, antigen binding proteins which comprise a variant CDR will bind to the same target protein or epitope with the same or similar binding affinity to the CDR, heavy chain, and/or light chain described herein.

The antibodies provided may also be formed into suitable pharmaceutical compositions, for administration to a human or animal patient to regulate FcYRIIa mediated conditions. Pharmaceutical compositions containing the antibodies provided, variations, and/or effective fragments thereof, may be formulated in combination with any suitable pharmaceutical vehicle, excipient or carrier that would commonly be used in this art, including such conventional materials for this purpose, e.g., saline, dextrose, water, glycerol, ethanol, other therapeutic compounds, and combinations thereof. The particular vehicle, excipient or carrier used will vary depending on the patient and the patient's condition, and a variety of modes of administration would be suitable for the compositions, as would be recognized by one of ordinary skill in this art. Suitable methods of administration of any pharmaceutical composition disclosed in this application include, but are not limited to, topical, oral, anal, vaginal, intravenous, intraperitoneal, intramuscular, subcutaneous, intranasal and intradermal administration.

The antibody compositions provided will thus be useful for interfering with, modulating, or inhibiting binding interactions between the FcYRIIa protein and its ligand protein prothrombin in blood and tissues, and will thus have particular applicability in developing compositions and methods of preventing or treating staphylococcal infection, and in inhibiting the activation of prothrombin.

The antibody compositions may be multi-specific, for instance, bispecific or trispecific, for instance including a portion that selectively binds platelets.

Accordingly, administration of the antibodies disclosed herein in any of the conventional ways described above (e.g., topical, parenteral, intramuscular, etc.), may provide an extremely useful method of treating or preventing certain conditions in human or animal patients. By effective amount is meant that level of use, such as of an antibody titer, that will be sufficient to bind FcYRIIa and/or to inhibit binding of another molecule to FcYRIIa, for instance, through steric inhibition or directly blocking the binding site of the other molecule and thus be useful in platelet regulation. As would be recognized by one of ordinary skill in this art, the level of antibody titer needed to be effective in treating or preventing staphylococcal infection will vary depending on the nature and condition of the patient, and/or the level of expression of FcYRIIa.

In addition to the use of disclosed antibodies and degenerative or homologs thereof as described above, we contemplate the use of these antibodies in a variety of ways, including the detection of the presence of FcYRIIa in diagnosis or in formulating treatment plans and options. Following isolation of a sample, diagnostic assays utilizing the disclosed antibodies may be carried out to detect the presence of FcYRIIa, and such assay techniques for determining such presence in a sample are well known to those skilled in the art and include methods such as, flow cytometry, radioimmunoassay, Western blot analysis and ELISA assays.

Accordingly, disclosed antibodies may be used for the specific detection or diagnosis of FcYRIIa mediated conditions, for the prevention or inhibition of conditions mediated by FcYRIIa, for diagnosis, or for use as research tools. The term “antibodies” as used herein includes monoclonal, polyclonal, chimeric, single chain, bispecific, simianized, and humanized or primatized antibodies as well as F(ab) fragments, such as those fragments which maintain the binding specificity of the antibodies to the FcYRIIa protein, including the products of an F(ab) immunoglobulin expression library. Accordingly, also contemplated is the use of single chains such as the variable heavy and light chains of the antibodies as will be set forth below. Generation of any of these types of antibodies or antibody fragments is well known to those skilled in the art.

In some embodiments, antibodies provided herein detect reactive platelets. Reactive platelets may comprise a determinant of thrombosis. Without wishing to be bound by any theory, measurement of FcYRIIa with the antibodies provided herein may inform subsequent therapeutic treatments, including treatment of thrombosis. For example, measurement of FcYRIIa antibody may be used to inform the use of blood thinners, aspirin, anticoagulants, surgery and/or other medications.

### Design of a Test Device

The test device can take any form desired that provides for the flow of a liquid test sample from the point of contact with the test sample past the test and/or control sites. In general, the test device of the present disclosure includes an interior flow pathway that includes one or more liquid permeable materials. In a first portion, the device includes a site for the application of a liquid sample. This first portion of the device also includes an analyte-binding conjugate, such as an antibody that specifically binds an antigen of interest (e.g., FcγRIIa, platelet surface proteins). The analyte binding conjugate typically binds the analyte to form a complex. Complex formation (e.g., formation of an antigen/antibody conjugate complex) may occur at any point in the interior flow pathway after the analyte contacts the analyte-binding conjugate. For example, complex formation may occur or continue as the sample flows from the first portion to the second portion of the device.

The second portion of the device has a variety of features that enhance functionality. In one embodiment, the second portion is composed of a material capable of filtering the sample to prevent the flow of particulate matter through the device. In another embodiment, the second portion facilitates complex formation by increasing the time required for the liquid to flow from the site of application to the test site. Accordingly, the dimensions of the second portion may be altered (e.g., increased or decreased) to empirically determine for each application those dimensions that enhance sensitivity while reducing false positives, i.e., optimizing the signal-to-noise ratio. In yet another embodiment, the second portion of the device can be used to deliver a desired agent to the liquid as it flows through the device. For example, the second portion may be impregnated with a buffer (e.g., TRIS, sodium carbonate), surfactant (e.g., Tween, Triton), preservative (e.g., Na azide, thimerosol), salt, or other agent, such that contact of the sample with the second portion of the device alters the sample. Exemplary alterations include an increase or decrease in the pH of the sample, in the salt concentration, in the buffering capacity, or in the binding between the conjugate and the analyte.

The third portion of the device includes a test site, which acts as a readout zone that provides for detection of an analyte in the sample. Various means for detecting the presence of an analyte at a test site are known in the art. In a competitive assay, a labeled probe competes with an analyte of interest for binding to a detector at the test site. The more analyte that is present in the sample, the more effectively it will be able to compete with, and/or displace, the binding of a detector. The hallmark of most competitive assays is that an increase in the amount of analyte in the sample results in a decrease of signal in the readout zone. In contrast, a “sandwich” format typically involves mixing the test sample with a detection probe conjugated with a specific binding member (e.g., antibody). In this embodiment, the conjugate and the analyte bind to form a complex. These complexes are then allowed to contact a receptive material (e.g., antibody) that is immobilized at the test site. The analyte/conjugate complex binds to the immobilized receptive material to form a “sandwich complex” (e.g., antibody conjugate/antigen/antibody). In this approach, detection of the “sandwich complex” indicates the presence of analyte in the sample.

It may be desirable to include a positive control to indicate that the liquid sample has traversed the interior flow path from the site of application past the test site. In a competitive assay format, the first portion of the device further includes a control conjugate and the third portion of the device includes a control site with a receptive material that binds the control conjugate. The control site is situated in the third portion of the device downstream from the test site. Detection of control conjugate binding at the control site indicates that the liquid sample flowed from the application site past the test site to the control site. In a sandwich assay format, a control antibody that binds the anti-antigen antibody is fixed at the control site. In the presence or absence of an antigen, excess anti-antigen antibody is detected at the control site.

The device may also include in a fourth portion a wicking pad that contains sorbent material capable of absorbing or adsorbing excess liquid present in the liquid sample.

In one embodiment, the test device contains a liquid permeable material defining the following portions in capillary communication:
a) a first portion that is the site for application of a liquid sample, comprising a liquid permeable medium, an anti-antigen antibody conjugate and a control antibody conjugate, where the first portion is between 5 mm and 20 mm in length; for example, the length of the first portion is equal to any integer between 5 and 20 (5, 10, 15, 20 mm in length);
b) a second portion comprising a liquid permeable medium, where the second portion overlaps the first portion by at least 1, 2, 3, 4, or 5 mm; and the length of the second portion is between 10 mm and 40 mm; for example, the length of the second portion is any integer between 10 and 40 (e.g., 10, 15, 20, 25, 30, 35, 40); and
c) a third portion that is the site for detecting the binding of the anti-antigen antibody conjugate at a test site and the binding of the control antibody conjugate at a control site, the third portion comprising a liquid permeable medium having the antigen fixed to the medium at the test site, and having an antibody that binds the control antibody present at a control site, wherein the third portion is between 15 and 40 mm in length; for example, is any integer between 15 and 40 (e.g., 15, 20, 25, 30, 35, 40); and the second portion overlaps the third portion by at least 1, 2, 3, 4, or 5 mm.

In a fourth portion the device may contain sorbent material. The sorbent material has a length between 25 and 75 mm. For example, the length is an integer between 25 and 75 (e.g., 25, 35, 50, 60, 70, 75). In one embodiment, the fourth portion overlaps the third portion by at least 1, 2, 3, 4, or 5 mm.

In general the interior flow path is between 1 mm and 10 mm in width; for example, the width of a test device (e.g., test strip) is any integer between 1 and 10 (e.g., 1, 2, 3, 4, 5, 6, 7, 8, 9, 10). In one embodiment the width of the strip is 3.8 mm. Desirably, a test device of the disclosure has increased sensitivity relative to a conventional test device. Sensitivity of a test device of the disclosure is increased by at least 5%, 10%, 25%, 50%, 75%, 100%, 150%, or 200% relative to a conventional test device.

As described herein, the test device includes a conjugate that binds an analyte. In one approach, the conjugate is an antibody capable of binding FcγRIIa, either alone or when conjugated to another compound. Any antibody, antibody conjugate, or fragment thereof that binds an antigen of interest may be used in the present disclosure. Such antibodies or antibody conjugates are present within the interior flow path of the test device. Suitable antibodies include, but are not limited to, polyclonal antibodies, monoclonal antibodies, or fragments thereof.

To detect the antibody/antigen complex within the test device, a detector reagent, or conjugate, must be coupled to the antibody or antigen. Exemplary conjugates include colored reagents, fluorescent compounds, enzymes, and radioactive isotopes. Colored or fluorescent compounds include gold particles, colored or fluorescent latex particles, polystyrene beads, and dyes, such as fluorescein isothiocyanate, BODIPY FL, Oregon Green, Alexa Fluor 488, phycoerythrin and phycocyanin. Colloidal metals, metal sols and other types of colored particles useful as marker substances in immunoassay procedures are known in the art. See, for example, U.S. Pat. No. 4,313,734. Antibody conjugates are widely available, for example, from a variety of well-known commercial sources (e.g., Molecular Probes (e.g., Zenon® labeling technology), Nanoprobes (e.g., Nanogold® Gold-Antibody Conjugates). The preferred label used as a detector reagent is the fluorochrome phycoerythrin (“PE”).

Enzymes that may be coupled to an antibody include peroxidases (such as horseradish peroxidase), phosphatases (such as acid or alkaline phosphatase), β-galactosidase, urease, glucose oxidase, carbonic anhydrase, acetylcholinesterase, glucoamylase, lysozyme, malate dehydrogenase, glucose-6-phosphate dehydrogenase, β-glucosidase, proteases, pyruvate decarboxylase, esterases, luciferase, or any other enzyme known to the skilled artisan. Enzymes are not in themselves detectable but must be combined with a substrate to catalyse a reaction the end product of which is detectable.

Antibodies, antibody conjugates, protein-antigen conjugates, and protein-hapten conjugates are fixed within the interior flow path using standard methods known to the skilled artisan. Protein immobilization protocols are known to the skilled artisan. See, for example, Laboratory Techniques in Biochemistry and Molecular Biology, Tijssen, Vol. 15, Practice and Theory of Enzyme Immunoassays, Chapter 13, The Immobilization of Immunoreactants on Solid Phases, pp. 297-328, and the references cited therein. In one approach, an antibody is immobilized directly on a solid support by physical adsorption or is bound covalently or through bridging molecules such as protein A, polylysine or to a solid support.

### Interior Flow Path

A test device may be provided that comprises an interior flow path that facilitates the flow of a liquid sample through the device. This interior flow path contains one or more liquid permeable materials or membranes composed of any relatively inert material or a combination of materials suitable for transporting a liquid (e.g., glass fibers, polyester, nitrocellulose, fibers of cellulose or derivatives thereof, non-cellulose hydrocarbon materials, ceramics) from the contact site past the test and/or control sites and, optionally, into a reservoir. Suitable materials for use in the interior flow path are wettable and exhibit low non-specific binding. Materials having increased sorptivity promote the flow of liquid. Different materials having different absorption characteristics or sorptivities may be used in various portions of the flow path. If desired, the materials to be used are screened for optimal pore size and density to facilitate the controlled distribution of an antibody within a membrane, to optimize reaction kinetics, or to optimize the sensitivity, discriminatory ability, or signal-to-noise ratio of the device.

### Solid Supports

For most applications, the test device includes an interior flow pathway fixed to a solid support. The physical shape of the solid support is not critical, although some shapes may be more convenient than others for the present purpose. Accordingly, the solid support may be in the shape of a paper strip, dipstick, membrane (e.g. a nylon membrane or a cellulose filter), a plate (e.g. a microtiter plate) or solid particles (e.g. latex beads). The solid support may be made of any suitable material, including but not limited to a plastic (e.g., polyethylene, polypropylene, polystyrene, latex, polyvinylchloride, polyurethane, polyacrylamide, polyvinylalcohol, nylon, polyvinyl acetate, or any suitable copolymers thereof), cellulose (e.g. various types of paper, such as nitrocellulose paper and the like), a silicon polymer (e.g. siloxane), a polysaccharide (e.g. agarose or dextran), or an ion exchange resin (e.g. conventional anion or cation exchange resins).

### Sorbent Reservoir

The test device optionally includes a fourth portion that forms a reservoir of adsorbent or absorbent material. This reservoir sorbs excess liquid as it flows through the test device. For some applications, such as where the concentration of antigen in a test sample is particularly low, it may be desirable to apply large volumes of a liquid test sample to the test device. In such cases, the presence of the adsorbent material may enhance the sensitivity of antigen detection. Optionally, the region of the flow path in the test cell defining the test and control sites is restricted in cross-sectional area relative to other regions of the flow path. This feature produces a “bottle-neck” effect wherein the antigen in the entire volume of adsorbed sample must pass through an area of restricted flow immediately above the test site. This “bottle-neck” may facilitate sandwich formation. Suitable sorbent materials include virtually any commercial material (e.g., synthetic or natural materials, such as cotton) capable of absorbing many times its weight in water. Such materials are widely available in commerce.

### Methods of Using the Test Device

The disclosure provides methods of using a test device of the disclosure for the detection of an analyte (e.g., an antigen) in a test sample. In one example, the assay is conducted by placing the leading edge (first portion) of a lateral flow device in contact with a liquid test sample. In another example, the sample is brought into contact with the device by applying a liquid test sample to the first portion of the lateral flow device in a drop-wise fashion.

### Test Samples

Methods and compositions of the disclosure are useful for the identification of an analyte (FcγRIIa) in a test sample. In one embodiment, the methods of the disclosure are suitable for detecting analytes of biological origin. Test samples include, but are not limited to, any liquid containing a dissolved or dispersed analyte (FcγRIIa) of biological origin. Exemplary test samples include body fluids (e.g. blood, plasma, amniotic fluid, sputum, urine, cerebrospinal fluid, lymph, tear fluid, feces, or gastric fluid), tissue extracts, or any liquid or biologic fluid containing a platelet. Exemplary conjugates that specifically bind platelets include without limitation antibodies to glycoprotein (GP) IIb (e.g., anti-CD41 or CD41a; antibodies to GP IIIa (e.g., anti-CD61); antibodies to GP V (e.g., anti-CD42d); antibodies to GP Ib (e.g., anti-CD42b); antibodies to GP IX such as anti-CD42a; antibodies to lysosomal membrane proteins (e.g., anti-CD63); antibodies to PECAM (e.g., anti-CD31). In various embodiments, a test device of the disclosure detects a FcγRIIa peptide or protein (e.g., on a platelet). If the test sample is not in itself sufficiently fluid for the present purpose, it may be admixed with a suitable fluid to the desired fluidity, for instance by homogenization.

### Kits

In another aspect, the disclosure provides kits for aiding in assessing platelet reactivity (e.g., determining a level of FcγRIIa expressed on platelets in a sample, identifying a subject having thrombosis or at risk of having thrombosis, selecting a treatment method for a subject having thrombosis or at risk of having thrombosis, and the like), which kits are used to detect biomarkers according to the disclosure. In one embodiment, the kit comprises agents that specifically recognize FcγRIIa. In specific embodiments, the agents are antibodies. Fluorescently labeled antibodies level are useful when flow cytometry methods are used to determine the level of FcγRIIa expressed on platelets in a sample. In a further embodiment, such a kit can comprise instructions for use in any of the methods described herein. In various embodiments, the instructions provide suitable operational parameters in the form of a label or separate insert. For example, the instructions may inform a consumer about how to collect the sample, how to wash the probe or the particular biomarkers to be detected, or how to determine platelet reactivity based on a measurement the level of FcγRIIa. In yet other embodiments, the kit can comprise one or more containers with controls (e.g., biomarker samples) to be used as standard(s) for calibration. In still other embodiments, the kit can comprise one or more therapeutic agents for the treatment of thrombosis (e.g., ADP receptor antagonists, PAR antagonists, and the like).

In additional embodiments, the disclosure provides kits that include a test device for the detection of an analyte in a sample. In one embodiment, the kit includes a lateral flow device described herein. In some embodiments, the kit comprises a container, which contains the lateral flow device; such containers can be boxes, ampoules, bottles, vials, tubes, bags, pouches, blister packs, or other suitable container forms known in the art. In one embodiment, such containers may be sterile. Such containers can be made of plastic, glass, laminated paper, metal foil, or other materials suitable for holding medicaments.

If desired the device is provided together with instructions for using it to identify the presence or absence of FcγRIIa in a sample. The instructions will generally include information about the use of the device for the identification of a particular analyte, such as FcγRIIa. The instructions may be printed directly on the container (when present), or as a label applied to the container, or as a separate sheet, pamphlet, card, or folder supplied in or with the container. If desired, the kit may also include a standard measure pipet, a test vial, and/or a liquid (e.g., ethanol, methanol, organic solvent, suitable buffer, such as phosphate buffered saline, or water) to be used in the extraction of a sample.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

### Assay Methodology

In another aspect, this disclosure provides an in vitro assay for the use of antibodies such as those disclosed herein. In this assay: 1) blood is anticoagulated with citrate or EDTA and fixed shortly after phlebotomy (advantageously this allows platelets to be stabilized and shipped to a laboratory for analysis, even up to and including approximately 1-2 weeks after the blood is drawn); 2) Fixed platelets are separated and washed; 3) Platelets are exposed to: (i) an antibody such as those described herein to quantify FcγRIIa and (ii) preferably also an antibody to identify platelets; and 4) After dilution, FcγRIIa may be quantified with the use of flow cytometry and the output may be standardized. Testing has demonstrated that this assay maintained excellent precision (coefficient of variation of repeated tests <5%). The assay will be of use in assessing and treating acute coronary syndrome, stroke and cancer.

Bangs Beads (Bang Laboratories, Inc.), Quantibrite™ (BD Biosciences) or other suitable fluorescent microspheres may be used to allow translation of fluorescence intensity units to molecules of FcγRIIa on the platelet surface. (Kay S, Herishanu Y, Pick M, et al. Quantitative flow cytometry of ZAP-70 levels in chronic lymphocytic leukemia using molecules of equivalent soluble fluorochrome. Cytometry B Clin Cytom. 2006; 70(4):218-226. Also, Quadrini K I, Hegelund A C, Cortes K E, et al. Validation of a flow cytometry-based assay to assess C5aR receptor occupancy on neutrophils and monocytes for use in drug development. Cytometry B Clin Cytom. 2016; 90(2):177-190.) In this way, platelet expression of FcγRIIa may be measured as the number of molecules of FcγRIIa/platelet.

A more detailed description of an exemplary assay methodology follows. A blood sample is drawn from a patient, for instance at a clinical site, into a standard citrate anticoagulated tube (sodium citrate liquid provided in the tube). Subsequently an aliquot of blood may be added to the fixative tube containing a formaldehyde or other suitable fixative. Optionally the sample tube may contain only an anticoagulant and a formaldehyde solution. This tube may be stored and may be shipped for analysis.

Processing of the sample may include the steps of:
Diluting sample with approximately 1,400 μl of HT buffer
Centrifuge for 2 minutes at 1,200 g
Decant liquid
Re-suspend platelet pellet in 1,000 μl HT buffer
Centrifuge for 2 minutes at 1,200 g
Decant liquid
Re-suspend platelet pellet in 1,000 μl HT buffer
Centrifuge for 2 minutes at 1,200 g
Re-suspend platelet pellet in 500 μl HT buffer
Assay tube final volume, typically approximately 30 μl, including
   2 μl of re-suspended platelet
   2 μg of 5G1 antibody or 11F9 antibody or other suitable antibody
   2 μl of CD42bPECy5 (Becton Dickinson)
   QS (quantity sufficient) with HT buffer
Incubate antibodies and platelets for 2 hours at room temperature
Dilute
Analyze with flow cytometry

Another method is provided for preparing a blood sample and includes the following steps:

A blood sample is treated with a suitable anticoagulant. Examples of suitable anticoagulants include those used for specimen collection, instruments, blood transfusions and equipment, as well as medical and surgical equipment. Examples include ethylenediaminetetraacetic acid (EDTA), citrate may be provided in liquid form in a sample tube, for instance, preloaded in the tube or in a blood transfusion bag or other device. Forms of citrate may include sodium citrate or acid-citrate-dextrose as well as certain oxalates (ethanedioates), for instance in salt or other suitable form.

A fixative, preferably of formaldehyde, may be added to a concentration of approximately 0.5% formaldehyde. Other suitable fixatives may be used, for instance, other aldehydes, such as glutaraldehyde. Certain alcohols may serve as a fixative, such as ethanol and methanol, as well as other known fixatives.

The blood sample may be incubated, for instance for 15 minutes, followed by dilution of the sample.

In one embodiment the sample is diluted to a fixative concentration such as 0.1%. In some embodiments, the fixative is diluted in a buffer. Typical buffers include phosphate buffered saline (PBS), Dulbecco's phosphate-buffered saline (DPBS), Hanks' Balanced Salt Solution (HBSS), HEPES and MOPSO. The buffer may comprise additional factors to aid in the stability of the sample. In some embodiments, the buffer will be combined with glycerol. Glycerol may be combined into solution with PBS creating a glycerol-buffer solution. This glycerol-buffer solution may comprise approximately 10% glycerol, 9% glycerol, 8% glycerol, 7% glycerol, 6% glycerol, 5% glycerol, 4% glycerol, 3% glycerol, 2% glycerol, 1% glycerol, 0.5% glycerol, 0.1% glycerol or any range in-between.

In some embodiments, the glycerol-buffer solution may be used to dilute a fixative. The fixative may be diluted with the glycerol-buffer solution at a ratio of approximately 1 (fixative):1(glycerol-buffer) solution, 1:2, 1:4, 1:8, 1:10, 10:1, 8:1, 4:1, 2:1 or any ratio in between.

In another embodiment, 100 μl of 1.5% formaldehyde fixative is added to a 2 ml screw cap tube. 200 μl of citrate anticoagulated whole blood is added so that the formaldehyde final concentration is approximately 0.5%. The mixture of fixative and citrate anticoagulated whole blood is gently mixed, for instance by flicking bottom of the tube a few times, and then incubated for 15 minutes at room temperature. 1.2 ml of phosphate buffered saline or other suitable diluent is then added, diluting the sample and effectively reducing the formaldehyde concentration to approximately 0.1%. The resulting mixture may then be mixed, for instance by inversion of the tube and then stored, for instance at approximately +4′C for later analysis and optionally shipment. Additional steps may be added.

Another method is provided for preparing a blood sample and includes the following steps:

Initial steps are performed (for instance at blood collection location). Blood is placed in a standard blue top tube, preferably a tube containing a 3.2% sodium citrate anticoagulant, which may be a vacutainer tube. This sample is then mixed to ensure effectiveness of the anticoagulant and 200 μl of blood is added to a collection tube containing 100 μl of fixative, for instance a formaldehyde fixative so that the formaldehyde final concentration is approximately 0.5%. In some embodiments, the fixative is diluted in buffer or a buffer-glycerol solution. The contents of collection tube may then be mixed, for instance as described above by tapping or flicking the bottom of the tube. The blood and fixative may then be incubated for 15 minutes at room temperature, and the sample diluted so that the formaldehyde final concentration is approximately 0.1%. After mixing the sample may be stored, for instance at approximately +4° C.

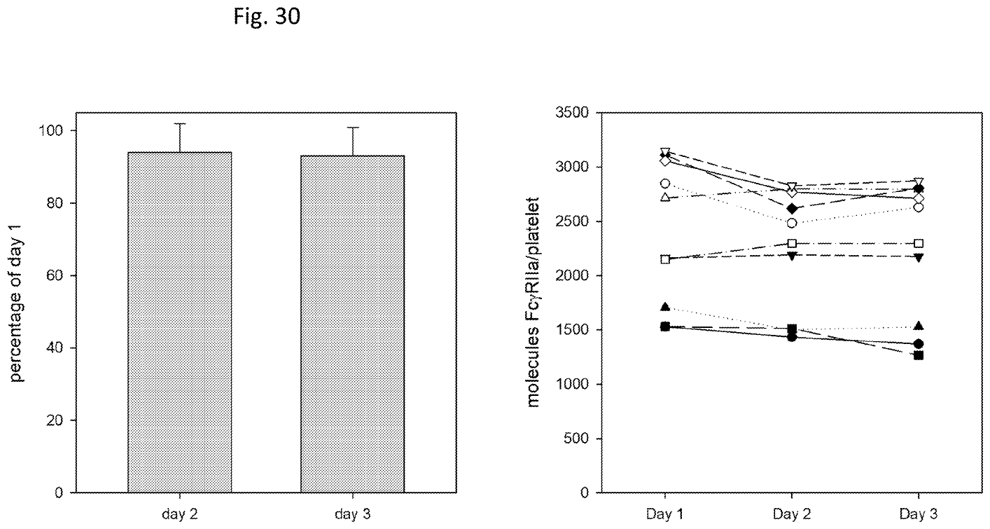

In some embodiments, blood can be fixed according to the methods provided herein one day, two days or up to three days after collection. FIG. 30 shows stability when anticoagulated blood is stored at room temperature up to three days.

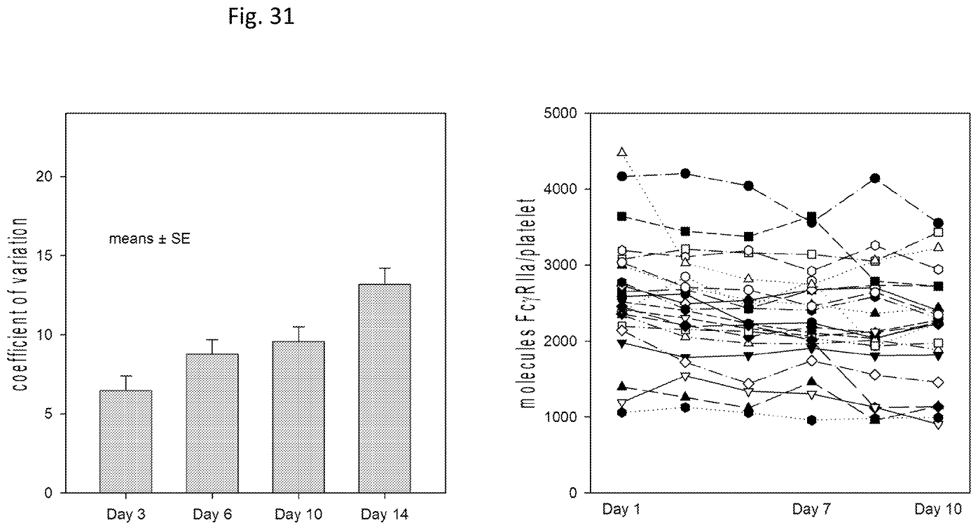

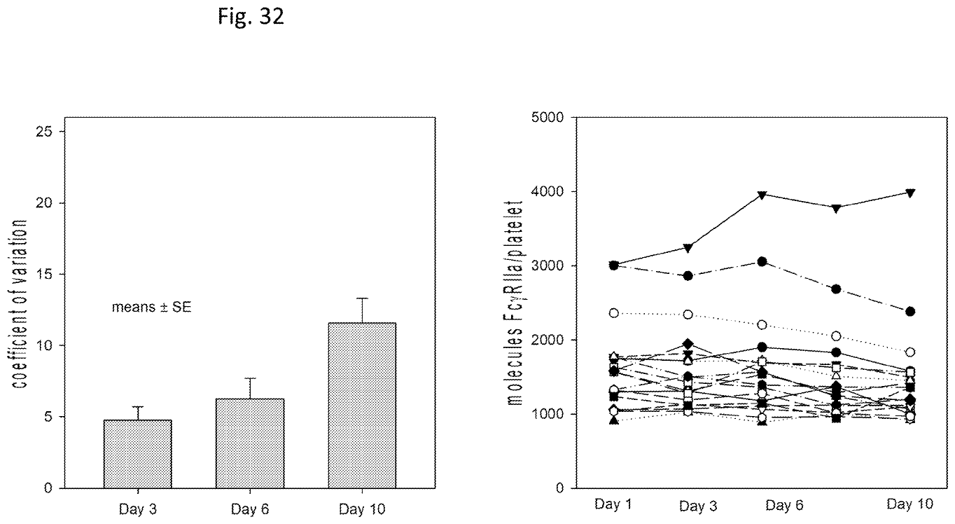

FIGS. 31 and 32 show the difference in stability over time after fixation with glycerol in the diluent.

Following storage, and, if necessary, shipment (optionally on wet ice), analysis of the sample may be performed, even at a facility remote from the blood draw location. The tube may be spun down in a centrifuge for 2 minutes at 1,200 g, the liquid decanted and the resulting pellet resuspending in phosphate buffered saline (“PBS”) and the full contents transferred to an Eppendorf tube. Further steps include:
Add 100 μl of ‘perm’ solution (ThermoFisher). This solution induces lysis of red blood cells with standard reagents that include either ammonium chloride or acetic acid
Ammonium chloride lyse (10× concentration)
NH₄Cl (ammonium chloride) 8.02 gm
NaHCO₃ (sodium bicarbonate) 0.84 gm
EDTA (disodium) 0.37 gm
QS to 100 ml with Millipore water.

Lysis of red blood cells can also be performed by the addition of 3% acetic acid (120 μl) to 5 μl of blood.
Incubate for 15 minutes
Add 800 μl PBS
Centrifuge for 2 minutes at 1,200 g
Decant liquid
Re-suspend platelet pellet in 1,000 μl PBS
Centrifuge for 2 minutes at 1,200 g
Decant liquid
Re-suspend platelet pellet in 1,000 μl PBS
Centrifuge for 2 minutes at 1,200 g
Re-suspend platelet pellet in 800 μl PBS with 0.2% BSA and store platelets at room temperature for at least 1 hour before assay (blocking step)
Assay tube final volume=35 μl, adding:
5 μl of re-suspended platelet
0.5 μg of 5G1-PE antibody
2 μl of CD42bPECy5 (Becton Dickinson)
QS with PBS/BSA 0.2%
Incubate antibodies and platelets for 1 hours at room temperature
Dilute with 500 μl of PBS
Analyze with flow cytometry

### Flow Cytometry Methods

Analyze platelet samples using template 032522 FCquant 5G1R-PE 375V_T. Set acquisition volume to 200 ul and run samples at 200 ul/min. count 10,000 events in platelet gate (CD42b positive events).
Platelets are first gated on a FSC SSC plot
A second gate for CD42b PECY5 positive events is created
A histogram plot gated on the CD42b positive events is created. A gate is set to the right of a non-immune IgG control. The x-mean of the histogram gate positive for 5G1R-PE binding is reported
Assay tube for IgG control, adding:
5 μl of re-suspended platelet
2 μl of IgG-PE
2 μl of CD42bPECy5 (Becton Dickinson)
26 μl PBS/BSA 0.2%

### Standardization of Flow Cytometry Output

By standardizing the flow cytometry output measurements, samples can be measured using different instruments, even at different labs, and on different days or times and under different conditions. Standardization allows for quantification of the number of molecules of FcγRIIa per platelet rather than relying on a measurement of mean fluorescence. Issues with reliance on mean fluorescence alone include results not being reproducible due to differences in, for instance, individual instruments or reaction conditions.

By standardizing the mean fluorescence measurements from a flow cytometry instrument, the flow cytometry output measurements can be converted into reliable, reproducible data, for instance the number of antibody molecules bound per cell. The standardization process uses beads with known ratios of phycoerythrin (“PE”) molecules to beads allowing the instrument or user to create a curve, histogram, or other tool or indicator that allows a determination of number PE molecules against measured fluorescence outputs. Binding antibodies conjugated with PE and running an assay sample through the flow cytometry instrument at the same settings then allows one to covert the measured fluorescence outputs to the number of antibodies bound. When antibody is bound to PE in a 1:1 ratio, the number of PE molecules per platelet is the number of copies of FcγRIIa per platelet.

A detailed method using Quantibrite™ is described below.
Remove 1 Quantibrite™ tube (lyophilized pellet of beads conjugated with four levels of phycoerythrin (PE) available from Beckton Dickinson™) in foil packet from fridge
Add 500 ul PBS/BSA 0.2% to Quantibrite™ tube. Mix gently.

Analyze beads in flow cytometer using the same voltage settings as for the platelet analysis, using template Quantibrite™ BD 375V_T. Run beads at 200 ul/min and count 20,000 events. In this way, measurements of samples with known levels of fluorescence markers (here PE) can be taken and used to standardize the output measurements for samples with unknown levels of fluorescence markers.

In particular, measurement of fluorescence with PE labeled-beads having known levels of PE allows one to establish a curve useful to convert mean fluorescence measurements into actual numbers of molecules of FcγRIIa per platelet.

Flow cytometry output (mean fluorescence intensity) may also be standardized with the use of Bang's Beads to enable specific quantification of the number of molecules of FcγRIIa per platelet.

One goal of the research described herein was to extend the time between phlebotomy and performance of the assay, without negatively affecting the assay. This means the antibody should still bind FcγRIIa on the platelets and an appropriately accurate measurement of FcγRIIa/platelet can still be achieved. Preferably the resulting measurement of FcγRIIa/platelet after a delayed time period before running the assay remains relatively close the measurements achieved after a short or zero delay before running the assay. In preferred embodiments the variation between FcγRIIa/platelet measurements is no more than 15%, or no more than 10%, or no more than 5% of measurements including 7, 10, 14, 21 days of delay before the assay vs. after only a short or zero delay between phlebotomy and performance of the assay. The increase in time between phlebotomy and performance of the assay allows for useful flexibility in performing the assay. For instance, the sample may be fixed and then stored or shipped before the assay is performed.

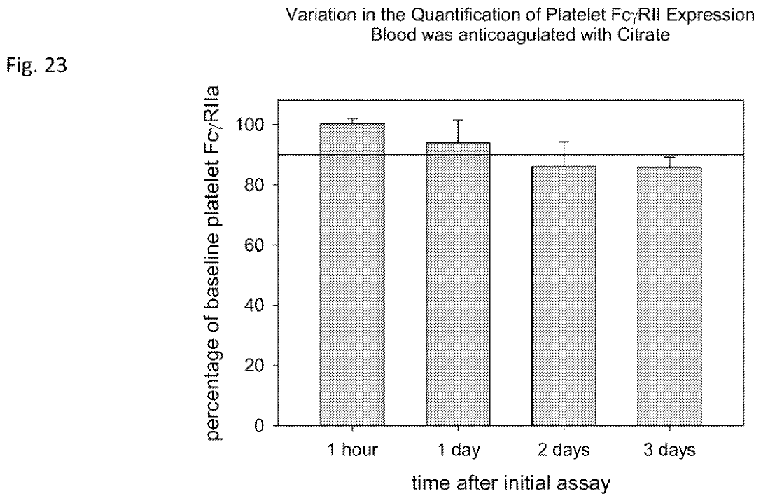

In another aspect the disclosure reflects the discovery that particular steps in preparing the platelet sample impact the stability of the sample and ability to store the sample before performing the assay. FIG. 23 shows results from varying the time period between blood draw and performance of the assay to quantification of platelet FcγRIIa, in tests where blood was stored in citrate anticoagulant. As shown, the measured levels of platelet FcγRIIa reduced with each day of storage. The line shows the 10% mark.

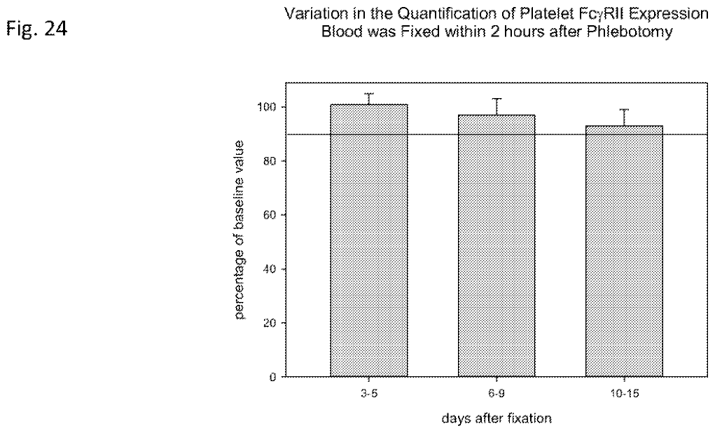

Remarkably, it was discovered that starting with a low concentration of fixative, for instance, formaldehyde (0.5%), waiting 15 minutes, and then diluting the concentration in the sample to 0.1% formaldehyde allows accurate quantification of platelet FcγRIIa even when the sample is stored for up to 2 weeks after blood was drawn and platelets were fixed, while maintaining less than 10% variation in measured platelet FcγRIIa compared with platelet FcγRIIa measured by assay performed immediately after blood was drawn. See FIG. 24. The line shows the 10% mark and that even after as long as 10-15 days following fixation, the level of platelet FcγRIIa measured had not dropped below 10% of the early 2-5 day measurements.

Preferably the blood sample is treated with the fixative at two hours or less after being drawn from the human patient into the blood sample tube.

In preferred variations, certain steps of a method of preparing a blood sample are performed. These steps of the method include fixation of the blood sample with a fixative at a concentration of 1.0-2.5% incubating to allow fixation, then reducing the fixative concentration, for instance to 0.5%, and then optionally storing the blood sample for 1-14 days, and further processing the sample.

Suitable methods may include:
adding a fixative to the blood sample at a concentration of fixative by weight percent of about 0.1-2.5%, preferably about 0.2-2.0% or about 1.0-1.5%, more preferably about 0.5% and preferably 0.5%.

Suitable concentrations may also include about 2.5%, 2.0%, 1.5% and 1.0%. Preferred fixatives are formaldehyde or a formaldehyde-based fixative.
incubating with the fixative for from 5-70 minutes, preferably from 5-60 minutes, 10-30 minutes, 10-20 minutes and most preferably 15 minutes.
diluting the sample with an appropriate buffer, for instance to about range of from about 0.2%-0.02%, or about 0.5%-0.05%, preferably to about 0.1% or 0.1% fixative. Optionally, the sample may be washed to bring the fixative to an amount below 0.02%.

Optionally, the sample may be stored, for instance at 4° C., or at room temperature, for a period of from about 1 day to about 21 days, preferably a period of from 1 day to about 14 days, most preferably 1 day to 14 days.

Following optional storage, and, if necessary, shipping, an antibody assay may then be performed, optionally include removal of formaldehyde (centrifuge to pellet platelets and 2 washes with PBS).

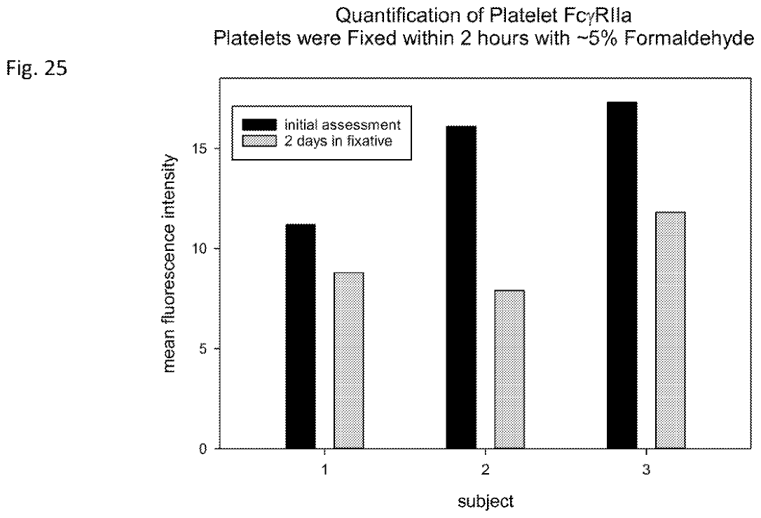

FIG. 25 relates to test results with approximately 5% formaldehyde fixative. Tests were performed on blood from generally low-risk human subjects (not shown). Such subjects generally have lower platelet FcγRIIa expression. In those subjects, fixation with approximately 5% formaldehyde within two hours showed less than 10% variation in results (not shown). Subsequent analytic variation was performed with platelets from subjects with higher expression (shown). In patients with higher platelet expression of FcγRIIa, a substantial decrement in mean fluorescence intensity was observed after 2 days of storage.

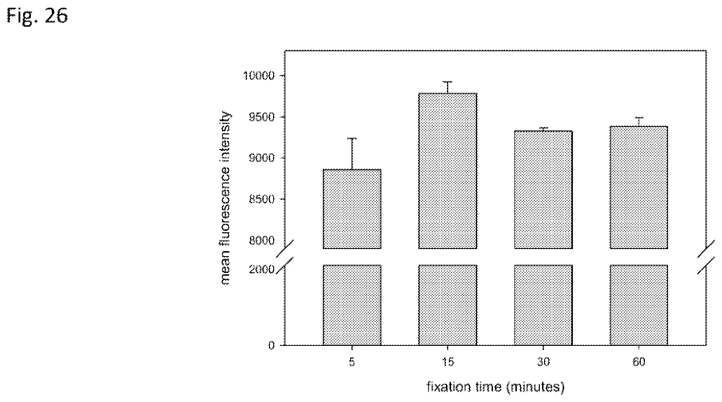

FIG. 26 shows results of testing based on varying the duration of time that platelets were fixed. In this testing, platelets in an in vitro citrate anticoagulated blood sample were fixed by introducing a low concentration of fixative to the sample, here formaldehyde at 0.5%, incubated for the chart-indicated time interval in minutes (5, 15, 30, or 60), and then diluting the fixative concentration in the sample to 0.1%. Mean fluorescence intensity based on FcγRIIa expression was measured in accordance with the procedures described herein. As shown, fixative incubation periods of 15 minutes and longer resulted in significantly reduced standard deviation across test results, as indicated by the error bars.

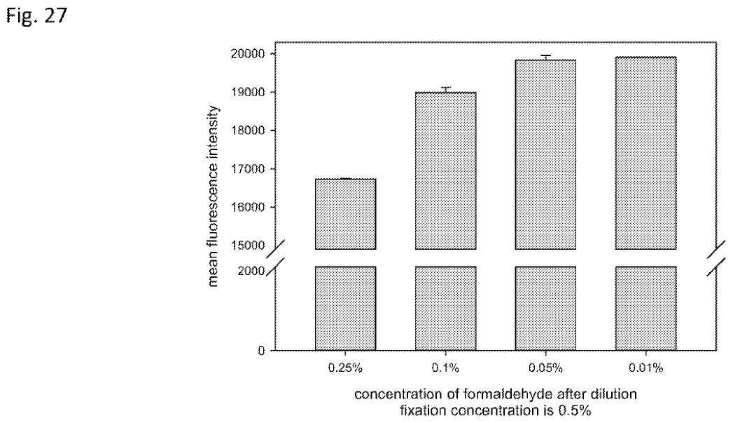

FIG. 27 shows results of testing to determine the impact of fixative dilution prior to sample storage. In this testing, platelets in an in vitro citrate anticoagulated blood sample were fixed by introducing a low concentration of fixative to the sample, here formaldehyde at 0.5%, incubated for 15 minutes, and then the sample was diluted with PBS to the chart-indicated concentration of formaldehyde fixative (0.25%, 0.1%, 0.05%, or 0.01%) and stored for 4 days before testing for FcγRIIa expression. FcγRIIa was quantified 4 days after fixation/dilution. The resulting measurements of FcγRIIa expression (as indicated by mean fluorescence intensity) showed a correlation between mean fluorescence intensity and increased dilution of the formaldehyde fixative. In particular, significantly lower measurements of mean fluorescence intensity were shown for the samples where the concentration of formaldehyde fixative was only reduced to 0.25%, as compared with higher measurements of mean fluorescence intensity for the samples where the concentration of formaldehyde fixative was more diluted, for instance to 0.1%, 0.05%, or 0.01%. These results show that dilution of the fixative impacts the ability to accurately measure FcγRIIa expression, particularly with dilution to 0.1%, 0.05%, or 0.01%.

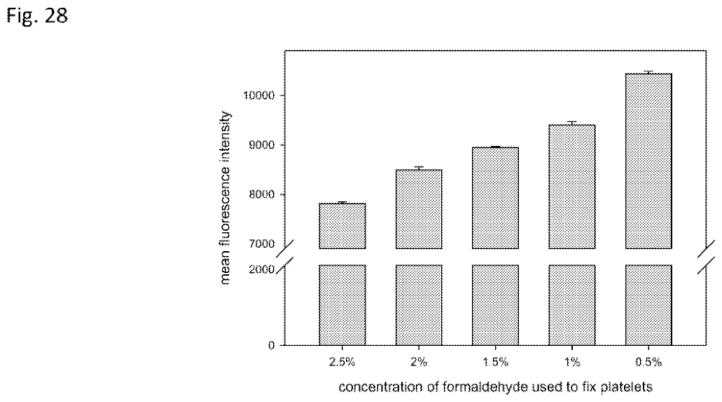

FIG. 28 shows results of testing to determine the impact of the concentration of formaldehyde fixative on quantification of FcγRIIa expression. In this testing, platelets in an in vitro citrate anticoagulated blood sample were fixed by introducing formaldehyde fixative to the concentration level indicated in the chart (2.5%, 2%, 1.5%, 1%, or 0.5%) for a period of 15 minutes, within 2 hours of sample collection. Subsequently, each sample was diluted to a final formaldehyde concentration of 0.1%. FcγRIIa expression assays were performed 4 days after fixation. The results showed an inverse correlation between the concentration of formaldehyde fixative used during the 15 minute formaldehyde fixation step and the resulting measurements of FcγRIIa expression (as indicated by mean fluorescence intensity). The results for 0.5% showed a significant improvement in the resulting measurements of FcγRIIa expression.

Previously known tests of platelet function were often influenced by platelet count. In contrast, the innovative methods described herein limit the impact of platelet count. Flow cytometry assesses each particle that falls into the selected gate. In the present context, platelets are identified by their size and the expression of a ubiquitous platelet marker. The mean fluorescence intensity is the average of all platelets counted (generally a group of at or about 10,000 platelets). Because each individual platelet is assessed, the measured expression is not influenced by platelet count.

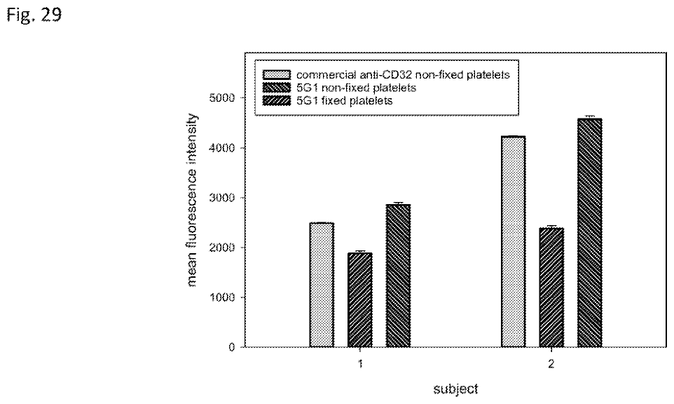

FIG. 29 shows results of testing for two different subjects using a commercially available FcγRIIa antibody as compared against using the 5G1 antibody described herein. The commercially available antibody is FLI8.26 monoclonal antibody from BD Pharmingen™. The test shows that with non-fixed platelets, the 5G1 antibody binds to a lesser extent than the commercially available antibody. But the 5G1 antibody binds at a higher level on fixed platelets. Remarkably, the results show that the 5G1 antibody disclosed herein binds to a greater extent to FcγRIIa on fixed platelets compared with FcγRIIa on non-fixed platelets.

Measurement accuracy and variability is known to increase when FcγRIIa expression is >25,000/platelet. In contrast, the innovative methods described herein ensure that the amount of antibody present is saturating. Greater variability associated with high expression suggests that the antibody is not saturating with high expression. This is addressed by increasing the concentration of antibody in the incubation. Additional steps in the assay may including repeating the assay after an additional dilution of platelets (effectively reducing the total amount of FcγRIIa and ensuring that the antibody is again saturating).

The innovative methods described herein provide an ability to accurately quantify FcγRIIa across a broad range of patient expression levels, for instance—1,500 to 30,000 molecules/platelet. Testing with microsphere beads has demonstrated the methods are capable of detecting from 500 to 42,000 molecules/bead. The in-vitro tests described herein may be applied on blood samples drawn from a human or animal. As would be appreciated by a person of skill in the art, when conducting the tests described herein, the blood sample being tested is preferably isolated from the human or animal patient. Additionally, the platelets may be isolated from the blood sample.

### Calibration of Flow Cytometry Output—Mean Fluorescence Intensity

Calibration of the flow cytometer may be and is preferably performed when the mean fluorescence intensity (MFI) for the medium low bead of the Quantibrite™ beads or Bang's MESF beads is outside the nominal expression ±5%.

Each flow cytometer instrument has individual characteristics. Additionally, results from a given flow cytometer instrument may vary, even on a day to day basis. To ensure that results between instruments are directly comparable, the output (MFI) may be synchronized. Standardization beads (Quantibrite™, MESF™) may be used to assess MFI. The medium low bead exhibits an MFI that is generally in the range of platelet expression of FcγRIIa. If the MFI of this bead is outside the nominal range ±5%, then calibration of the instrument is preferably performed before analysis of clinical samples. Each lot of beads may be compared with the previous lot to ensure that the measured signal remains stable. Similarly, each lot of Rainbow beads will be compared with previous lot to ensure a consistent signal.

### Materials

BD Rainbow Calibration Beads: BD cat #556286 (beads dyed to eight different fluorescence intensities and are excited across a range of wavelengths)
BD Quantibrite™; Fisher catalog #: 50-620-179 (standardization beads) or Bang's MESF Beads, Bang's catalog #827

### Procedure

Create a new experiment in the flow cytometer instrument
Set a forward scatter/side scatter (FSC/SSC) linear plot
Set 2 histogram plots, one for BL2 (phycoerytrin—PE) channel, and the other one for RU (PECy5) channel
In Instrument settings set the voltage that has been used for assessing Quantibrite™ Beads (as well as control and platelet samples). General starting points are a BL2 voltage of 400, and RU voltage of 350
Set threshold at 25
Add 3 drops of vortexed Rainbow Calibration beads to 1 ml phosphate buffered saline (PBS), and vortex before testing in flow cytometer
Set acquisition volume to 100 μl and flow rate to 25 μl/min.
Adjust the FSC/SSC voltage until beads are visible in FSC/SSC (approximately 350V for BL2 and 350V for RU)
Draw a gate around single bead population (R1) or name the gate
Set histograms on R1
Rerun the beads and collect 10,000 beads in R1
Draw 5 gates in each histogram plot and record the MFI
Adjust voltages for BL2 and RU channel until they fall in the ranges defined below
   PE channel
   R2: 10,500-11,500
   R3: 33,000-36,000
   R4: 105,000-115,000
   R5:315,000-345,000
   R6: 760,000-840,000
   PECy5 Channel
   R7: 3,000-3,600
   R8: 11,000-12,000
   R9: 36,000-39,000
   R10: 95,000-105,000
   R11: 160,000-176,000
Save voltage settings in the FcγRIIa Platelet Assay template that is used for Quantibrite™, control beads, and platelet samples.

### Preparation of High, Low, and Negative FcγRIIa Control Beads

In another method, FcγRIIa control beads are prepared for use in flow cytometry.

### Materials:

Bang's Polystyrene Microspheres 2.36 μm diam. catalog #: PC05002
Bang's PolyLink Protein Coupling Kit. Cat #PL01N

Preferably control beads having three different levels of FcγRIIa are prepared:
Negative control bead (BSA control): zero FcγRIIa
Low FcγRIIa bead: 0.5 μg FcγRIIa: 125 μl of bead solution
High FcγRIIa bead: 4 μg FcγRIIa: 125 μl of bead solution.

Example procedure for preparing low and high control beads:
Warm microparticles, coupling buffer (available from Bang's), and wash/storage buffer (available from Bang's) to room temp. The coupling buffer is designed to activate the beads and prepare them for covalent linking. Common buffers include MES (2-(N-morpholino)ethanesulfonic acid) buffer, (MES Buffer), pH range 5.7 to 7.2
Dissolve 19.2 g of MES free acid (MW 195.2) in ˜900 mL of pure water. Titrate to desired pH with 1 N HCl or 1 N NaOH and adjust final volume to 1000 mL with pure water
Vortex beads on high speed 4 times for ˜5 seconds each before pipetting 125 μl into a microcentrifuge tube
Pellet beads in microcentrifuge (3 min @1200 g)
Aspirate supernatant, and wash beads by adding 400 μl coupling buffer (CB)
Pellet beads again (centrifuge for 3 min @1200 g)
Aspirate supernatant, and resuspend beads with 170 μl CB, transfer to 2 ml microcentrifuge tube. Sonicate beads (maximum setting) for 60 seconds
Prepare EDAC (1-Ethyl-3-(3-dimethylaminopropyl) carbodiimide Hydrochloride) solution: EDAC is preferably stored at −20° C. and should be freshly prepared just before addition to beads. Weigh out approximately 10 mg of EDAC and QS to 200 mg/ml with CB
Add 20 μl to the 170 μl microparticle suspension while vortexing on medium speed for ^{˜}5 seconds and allow the activation step to proceed for 15 minutes
Sonicate (max setting) sample for 1 minute
Dilute FcγRIIa protein in 100 μl CB and add to activated beads while vortexing on medium speed
Mix end over end for 30 minutes. Sonicate (max setting) the sample 1 minute after 15 minutes and 30 minutes for 60 seconds
Add 50 μl of 1% BSA/PBS to tube and continue incubation for 15 minutes
Vortex at high speed for ^{˜}5 seconds
Pellet beads (centrifuge for 5 min @1200 g)
Aspirate supernatant and resuspend beads with 400 μl Wash/Storage buffer (WSB), vortex for ^{˜}10 seconds
Pellet beads (centrifuge for 5 min @1200 g)
Aspirate supernatant and resuspend beads with 400 μl W/SB.
Vortex on high speed for ^{˜}10 seconds
Add 100 μl 1% BSA in W/SB. Final concentration of BSA is 0.2%, final volume is 500 μl
Analyze 1 μl with flow cytometers to assess for single bead suspension. Sonicate beads for 2 minutes if a substantial amount of aggregates are apparent

### Procedure for BSA Negative Control Beads:

BSA negative control beads are saturated with BSA by absorption. This prevents non-specific binding of the FcγRIIa antibody
Pipette 125 μl of vortexed beads into a microcentrifuge tube
Pellet beads in microcentrifuge (3 min @1200 g)
Aspirate supernatant, and wash beads by adding 400 μl coupling buffer (CB)
Pellet beads (centrifuge for 3 min @1200 g)
Aspirate supernatant, and resuspend beads with 400 μl 1% BSA W/SB
Vortex beads at high speed for ^{˜}5 seconds and mix end over end for 1 hour
Sonicate beads (max setting) for 1 minute after 30 minutes and at end of incubation
Pellet beads (centrifuge for 5 min @1200 g)
Aspirate supernatant and resuspend beads with 400 μl W/SB
Vortex on high speed for ˜10 seconds
Add 100 μl 1% BSA in W/SB. Final concentration of BSA is 0.2%
Analyze 1 μl with flow cytometers to assess for single bead suspension. Sonicate beads for 2 minutes if a substantial amount of aggregates are apparent

### Assay for Quantification of Platelet FcγRIIa Expression

### Key Commercial Reagents

ThermoFisher Fix Medium A; ThermoFisher catalog #GAS001S100
ThermoFisher Permeabilization Medium B; ThermoFisher catalog #GAS002S5
Phosphate Buffered Saline (PBS); Fisher catalog #: 21031CM
Bovine Serum Albumin 10% in PBS (BSA); Fisher cat #: P137525
BD Quantibrite; Fisher catalog #: 50-620-179 or Bang's MESF Beads, Bang's catalog #827
CD42b PECY5; Fisher catalog #: BDB551141
Control IgG PE; Fisher catalog #: BDB340013

### Steps to be Performed at Clinical Site

Collect in-vitro blood sample into in a standard blue top (citrate) tube or other suitable container
Mix blood in blue top tube before adding 200 μl of blood to collection tube (for instance a screw top tube with colored cap). Collection tube contains 100 μl of Fix that is diluted 1:4 in PBS
Mix contents of collection tube by tapping the bottom of the tube
Incubate blood with Fix for 15 minutes at room temperature
Transfer entire contents of Diluent (phosphate buffered saline) (1.2 ml) to the collection tube with the supplied transfer pipette.
Mix contents by inversion
Store collection tube at 4° C. until shipped
Ship tube on wet ice

### Steps to be Performed at Laboratory

Step 1: Calibration of Flow Cytometer: Preferably Performed Each Day that Assays are Performed

Each flow cytometer has individual characteristics. To ensure that results between instruments are directly comparable, the output (mean fluorescence intensity—MFI) is preferably synchronized. Standardization beads (Quantibrite™) will be used to assess MFI. The medium low bead has an MFI that is in the range of platelet expression of FcγRIIa. Each lot of Quantibrite™ beads is preferably compared with the previous lot to ensure that the signal remains stable. If the MFI of the medium low bead is outside the range of 27,000-30,000, then calibration of the instrument is preferably performed before analysis of clinical samples (follow calibration protocol).

### Preparation of Quantibrite™ Standard Curve

Remove 1 Quantibrite™ tube in foil packet from fridge
Add 500 μl PBS/BSA 0.2% to Quantibrite™ tube. Mix gently
Analyze beads in flow cytometer using the same voltage settings used for the platelet analysis. Run beads at 200 μl/min and count 20,000 events.
   Preparation of Bang's™ MESF R-PE standard curve (alternative to Quantibrite™)
Label 5 eppendorf tubes with B, 1, 2, 3, 4
Add 500 μl PBS to each tube.
Remove MESF beads from fridge, mix them gently by inversion and tapping the bottom of the tube.
Add 20 μl of Bang's MESF beads to the 500 ul PBS.
vortex tubes
Analyze beads in flow cytometer using the same voltage settings used for the platelet analysis.
   Step 2: Analyze Control Samples: To be Performed Each Day that Assays are Performed Analyze High and Low Control Beads in Duplicate. Assay Procedure:
Add 50 μl of 1% BSA/PBS to assay tube
Add 0.5 μg phycoerythrin labeled 5G1 antibody (5G1PE)
Add 1 μl of FcγRIIa control beads
Incubate beads with antibody for 30 minutes
Add 1 ml PBS, spin @1200 g for 3 minutes
Aspirate supernatant, and resuspend with 1000 μl PBS
Flow cytometry analysis should be performed 1 hour after wash

### Flow Cytometry Analysis of Control Beads

Control beads should be analyzed with the same settings as defined for Quantibrite™ and platelet assays
Create a forward scatter/side scatter (FSC/SSC) log plot. Gate around single bead population.
Create histogram plot on 5G1PE gated on single bead population gate.
Run labelled beads, setting a gate excluding the BSA peak (nonspecific binding), and record the MFI of the low and high control.
The MFI of the low and high control will be translated into molecules of FcγRIIa/bead using calculation template (described below).
If the high and low control values (molecules of FcγRIIa/bead) are outside the prespecified range (provided with beads), then calibration (Quantibrite™ and flow cytometry output) should be performed.

### Step 3: Platelet FcγRIIa Assay

Centrifuge collection tube for 2 minutes at 1,200 g
Decant liquid
Re-suspend platelet pellet in 100 μl PBS, transfer full content to an Eppendorf tube.
Add 100 μl of ‘perm’ solution
Incubate for 15 minutes
Add 800 μl PBS
Centrifuge for 2 minutes at 1,200 g
Decant liquid
Re-suspend platelet pellet in 1,000 μl PBS
Centrifuge for 2 minutes at 1,200 g
Decant liquid
Re-suspend platelet pellet in 1,000 μl PBS
Centrifuge for 2 minutes at 1,200 g
Re-suspend platelet pellet in 800 μl PBS with 0.2% BSA and store platelets at room temperature for at least 1 hour before assay (blocking step)
Assay tube final volume=35 μl
   5 μl of re-suspended platelet
   0.5 μg of 5G1-PE
   2 μl of CD42bPECy5 (Becton Dickinson)
   QS with PBS/BSA 0.2%
Incubate antibodies and platelets for 1 hour at room temperature
Add 50 μl of Medium A fixative and incubate for 15 minutes at room temperature
Dilute with 500 μl of PBS
Analyze with flow cytometry

### Flow Cytometry

Analyze platelet samples using template. Set acquisition volume to 200 μl and run samples at 200 μl/min. Count 10,000 events in platelet gate (CD42b positive events).
Platelets are first gated on size (FSC/SSC plot)
A second gate for CD42b PECY5 positive events is created
A histogram plot gated on the CD42b positive events is created. A gate is set to the right of a non-immune IgG control (51% of platelets are in gate). The MFI of the histogram gate that bind 5G1PE is reported
Assay tube for IgG control
   5 μl of re-suspended platelet
   2 μl of IgG-PE
   2 μl of CD42bPECy5 (Becton Dickinson)
   26 μl PBS/BSA 0.2%

### Calculation of Molecules FcγRIIa/Platelet

BD Quantibrite™ beads include 4 MFI peaks with a specific number of PE molecules/bead. These results will be used to create a calibration curve that translates MFI into molecules of FcγRIIa/platelet. A calculation template is provided.
The calculation template (Excel file) will be used to enter the MFI from the histogram statistics
Lot specific values of the Quantibrite™ PE molecules per bead will be used
A linear regression formula that relates Log PE molecules/bead to Log of the MFI will be generated
The slope and intercept of the standard curve will be calculated
The formula will translate MFI into molecules of FcγRIIa/platelet (or molecules of FcγRIIa/control bead).

### Additional Definitions

By “Adenosine diphosphate (ADP) receptor” is meant a purinergic G protein-coupled receptors, stimulated by the nucleotide Adenosine diphosphate (ADP). ADP receptors include P2Y12 which regulates thrombosis. Adenosine diphosphate (ADP) receptor antagonists are agents that inhibit adenosine diphosphate receptors. P2Y12 is the target of the anti-platelet drugs including prasugrel, clopidogrel, and other thienopyridines.

As used herein the term “about” means+/−5% of the stated value, except as stated otherwise, and always expressly includes the stated value.

By “clopidogrel” is meant (+)-(S)-methyl 2-(2-chlorophenyl)-2-(6,7-dihydrothieno[3,2-c]pyridin-5(4H)-yl)acetate which is a potent platelet aggregation inhibitor.

By “prasugrel” is meant (RS)-5-[2-cyclopropyl-1-(2-fluorophenyl)-2-oxoethyl]-4,5,6,7-tetrahydrothieno[3,2-c]pyridine-2-yl acetate which is a potent platelet aggregation inhibitor.

By “ticagrelor” is meant (1S,2S,3R,5S)-3-[7-[(1R,2S)-2-(3,4-Difluorophenyl)cyclopropylamino]-5-(propylthio)-3H-[1,2,3]triazolo[4,5-d]pyrimidin-3-yl]-5-(2-hydroxyethoxy)cyclopentane-1,2-diol which is a potent platelet aggregation inhibitor.

By “vorapaxar” is meant Ethyl N-[(3R,3aS,4S,4aR,7R,8aR,9aR)-4-[(E)-2-[5-(3-fluorophenyl)-2-pyridyl]vinyl]-3-methyl-1-oxo-3a,4,4a,5,6,7,8,8a,9,9a-decahydro-3H-benzo[f]isobenzofuran-7-yl]carbamate which is a potent platelet aggregation inhibitor.

By “anti-thrombotic therapy” is meant any treatment used to inhibit or reduce thrombosis or to inhibit or reduce platelet aggregation in a subject.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “analyte” is meant any compound under investigation using an analytical method.

By “analyte-binding conjugate” is meant a detectable molecule that binds a compound under investigation.

By “capillary communication” is meant facilitating the flow of a liquid between liquid permeable materials.

By “capture reagent” is meant a reagent that specifically binds a polypeptide or nucleic acid molecule to select or isolate the polypeptide or nucleic acid molecule. In various embodiments, the capture reagent for an FcγRIIa polypeptide is an anti-FcγRIIa antibody. In other embodiments, a platelet capture reagent specifically binds a platelet cell surface polypeptide (e.g., useful for binding platelets to a solid phase). Exemplary platelet capture reagents include without limitation antibodies to glycoprotein (GP) IIb (e.g., anti-CD41 or CD41a; antibodies to GP IIIa (e.g., anti-CD61); antibodies to GP V (e.g., anti-CD42d); antibodies to GP Ib (e.g., anti-CD42b); antibodies to GP IX such as anti-CD42a; antibodies to lysosomal membrane proteins (e.g., anti-CD63); antibodies to PECAM (e.g., anti-CD31).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Contacting” as used herein, e.g., as in “contacting a sample” refers to contacting a sample directly or indirectly in vitro, ex vivo, or in vivo (i.e. within a subject as defined herein). Contacting a sample may include addition of an antibody to a sample, a compound to a sample, or administration to a subject. Contacting encompasses administration to a solution, cell, tissue, mammal, subject, patient, or human. Further, contacting a cell includes adding an agent to a cell culture.

By “a control conjugate” is meant a detectable molecule that does not substantially bind a compound under investigation.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens, including, for instance, phycoerythrin.

By “disease” is meant any condition or disorder that damages or interferes with the normal function of a cell, tissue, or organ. Examples of diseases include thrombotic disease associated with an undesirable increase in platelet reactivity and/or the formation of a thrombus, such as a thrombus that results in an ischemic event.

By “effective amount” is meant the amount of an agent required to ameliorate the symptoms of a disease relative to an untreated patient. The effective amount of active compound(s) or composition used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “flow cytometry” is meant a technique for counting and examining microscopic particles that allows for multiparametric analysis of the physical and/or chemical characteristics of the microscopic particles.

As used herein, the terms fixed, fixation, fixative, and various forms thereof, refer to the treatment of biological cells to preserve their properties, or to prevent or at least slow decay or other degradation. In certain embodiments, fixing is aimed at terminating or slowing biochemical reactions and may also increase the treated cells' mechanical strength or stability. Formaldehyde fixation is preferred and useful in flow cytometry. A suitable alternative, glutaraldehyde, exhibits autofluorescence. In the embodiments described herein formaldehyde was used for fixation of platelets, unless stated otherwise.

The disclosure provides a number of targets that are useful for the development of highly specific drugs to treat a thrombotic disease or disorder characterized by the methods delineated herein (e.g., characterized by an undesirable increase in platelet reactivity). In addition, the methods of the disclosure provide a facile means to identify therapies that are safe for use in subjects. In addition, the methods of the disclosure provide a route for analyzing virtually any number of compounds for effects on a thrombotic disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “lateral flow device” is meant a test device that relies on the flow of a liquid via capillary action, wicking, or wetting a liquid permeable media present in the device.

By “liquid permeable material” is meant a material susceptible to wetting, wicking, or transport of a liquid by capillary action.

By “marker” is meant any protein or polynucleotide having an alteration in expression level or activity that is associated with a disease or disorder. For example, an increase in FcγRIIa level, activity, phosphorylation, or expression is associated with increased platelet reactivity and/or an increased propensity to develop a thrombotic disease or disorder.

As used herein to indicate ingredient amounts in compositions, percentages refer to weight percentages, except as otherwise indicated.

By “portion” is meant some fraction of a whole. A portion of a test device, for example, may be 0.1, 0.2, 0.3, 0.4, 0.5, 0.6, 0.7, 0.8, 0.9 of the length of the interior flow path of the device.

By “Protease-activated receptor (PAR)” is meant a G protein-coupled receptor that is activated by cleavage of a portion of its extracellular domain. PARs are highly expressed in platelets, including the thrombin receptors PAR1, PAR3 and PAR4. PARs are activated by the action of serine proteases such as thrombin (e.g., activating PARs 1, 3 and 4). Cleavage of the N-terminus of the receptor, generates a tethered ligand (SFLLRN) that acts as an agonist, causing a physiological response. The cellular effects of thrombin are mediated by protease-activated receptors (PARs). Thrombin signaling in platelets contributes to hemostasis and thrombosis. Thrombin receptor antagonists include Vorapaxar (SCH 530348) which is a PAR1 antagonist.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100% in a parameter.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. “Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 75%. Preferred percent identity of polypeptides can be any integer from 75% to 100%. More preferred embodiments include at least 75%, 80%, 85%, 86%, 87%, 88%, 89%, 90%, 91%, 92%, 93%, 94%, 95%, 96%, 97%, 98%, 98.7%, or 99%.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, (1981) Adv. Appl. Math. 2:482, by the homology alignment algorithm of Needleman and Wunsch, (1970) J. Mol. Biol. 48:443, by the search for similarity method of Pearson and Lipman, (1988) Proc. Natl. Acad. Sci. USA 85:2444, by computerized implementations of these algorithms (GAP, BESTFIT, FASTA, and TFASTA), or by visual inspection (see generally, Current Protocols in Molecular Biology, F. M. Ausubel et al., eds., Current Protocols, Greene Publishing Associates, Inc. and John Wiley & Sons, Inc., (1995 Supplement)).

Examples of algorithms that are suitable for determining percent sequence identity and sequence similarity are the BLAST and BLAST 2.0 algorithms, which are described in Altschul et al., (1990), J. Mol. Biol. 215: 403-410 and Altschul et al., (1977) Nucleic Acids Res. 3389-3402, respectively. Software for performing BLAST analyses is publicly available through the National Center for Biotechnology Information website.

### Similar DNA and Protein Sequences

Two DNA sequences are “substantially similar” when approximately 70% or more (e.g., at least about 80%, at least about or at 85% 89%, 90%, 91%, 92%, 93%, 94%, 95%, 96%, 97%, 98%, or 99%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., Molecular Cloning: A Laboratory Manual, 1982; DNA Cloning, Vols. I & II, supra; Nucleic Acid Hybridization, [B. D. Hames & S. J. Higgins eds. (1985)].

By “substantially similar” is further meant a DNA sequence which, by virtue of the degeneracy of the genetic code, is not identical with that shown in any of the sequences disclosed, but which still encodes the same amino acid sequence; or a DNA sequence which encodes a different amino acid sequence that retains the activities of the proteins, either because one amino acid is replaced with a similar amino acid, or because the change (whether it be substitution, deletion or insertion does not affect the active site of the protein.

Two amino acid sequences or two nucleic acid sequences are “substantially similar” when approximately 70% or more (e.g., at least about 80%, at least about or at 85%, 89%, 90%, 91%, 92%, 93%, 94%, 95%, 96%, 97%, 98%, or 99%) of the amino acids match over the defined length of the sequences.

As demonstrated by evidence herein, modification and changes may be made in the structure of the peptides and DNA segments which encode them and still obtain a functional molecule that encodes a protein or peptide with desirable characteristics. The changing the amino acids of a protein may be used to create an equivalent, or even an improved, second generation molecule.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, J. Mol Biol, 157(1):105-132, 1982.

It is known in the art that certain amino acids may be substituted by other amino acids having a similar hydropathic index or score and still result in a protein with similar biological activity, i.e., still obtain a biological functionally equivalent protein. In making such changes, there is a general trend toward substitution of amino acids whose hydropathic indices are within about ±0.5 to about ±2. It is also understood in the art that the substitution of like amino acids can be made effectively on the basis of hydrophilicity. For example, local average hydrophilicity of a protein, as governed by the hydrophilicity of its adjacent amino acids, may correlate with a biological property of the protein.

It is understood that an amino acid can be substituted for another having a similar hydrophilicity value and still obtain a biologically equivalent, and in particular, an immunologically equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include but are not limited to: arginine and lysine; glutamate and aspartate; serine and threonine; glutamine and asparagine; and valine, leucine and isoleucine.

The polypeptides can be chemically synthesized. The synthetic polypeptides are prepared using the well-known techniques such as but not limited to solid phase, liquid phase, or peptide condensation techniques, or any combination thereof, and can include natural and unnatural amino acids.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42′ C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30′ C. in 750 mM NaCl, 75 mM trisodium citrate, and 1% SDS. In a more preferred embodiment, hybridization will occur at 37′ C. in 500 mM NaCl, 50 mM trisodium citrate, 1% SDS, 35% formamide, and 100 μg/ml denatured salmon sperm DNA (ssDNA). In a most preferred embodiment, hybridization will occur at 42′ C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68′ C. In a preferred embodiment, wash steps will occur at 25′ C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68′ C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “subject” is meant a mammal, including, but not limited to, a human or non-human mammal, such as a bovine, equine, canine, ovine, or feline. In some embodiments the subject is at an increased risk of thrombosis. In some embodiments, the subject is suspected of having an increased risk of thrombosis or platelet reactivity or any condition associated with thrombosis or reactive platelets. The subject may also have or be suspected of having coronary artery disease, renal disease, or end stage genal disease, one or more myocardial infarctions, cardiovascular disease, stroke, diabetes, or atherosclerosis.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “test device” is meant a device used in the detection of an analyte in a sample.

By “wick” is meant to sorb a liquid.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Other Embodiments

In certain embodiments this disclosure relates to any of the following examples 1. An isolated antibody or antigen binding fragment thereof that specifically binds to FcγRIIa comprising at least one heavy chain complementarity-determining region (CDRH) and at least one light chain complementarity-determining region (CDRL), wherein the CDRH is selected from SEQ ID NO: 5, SEQ ID NO: 6 and SEQ ID NO: 7 and the CDRL are selected from SEQ ID NO: 10, SEQ ID NO: 11 and SEQ ID NO: 12 wherein SEQ ID NO: 11 encodes the amino acid sequence DTS, or wherein the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22 wherein SEQ ID NO: 21 encodes the amino acid sequence WAS.

2. The isolated antibody or antigen binding fragment thereof of example 1, comprising at least two heavy chain complementarity-determining regions (CDRH) and at least two light chain complementarity-determining regions (CDRL), wherein the CDRH is selected from SEQ ID NO: 5, SEQ ID NO: 6 and SEQ ID NO: 7 and the CDRL are selected from SEQ ID NO: 10, SEQ ID NO: 11 and SEQ ID NO: 12 or wherein the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22.

3. The antibody or antibody binding fragment thereof of example 1 or 2, comprising a CDRH1 region comprising SEQ ID NO: 5, a CDRH2 region comprising SEQ ID NO: 6, and a CDHR3 region comprising SEQ ID NO: 7; and a CDRL1 region comprising SEQ ID NO: 10, a CDRL2 region comprising SEQ ID NO: 11 and a CDRL3 region comprising SEQ ID NO: 12 and sequences at least 90% identical to SEQ ID NO: 5, SEQ ID NO: 6, SEQ ID NO: 7, SEQ ID NO: 10, SEQ ID NO: 11 and SEQ ID NO: 12; or a CDRH1 region comprising SEQ ID NO: 15, a CDRH2 region comprising SEQ ID NO: 16, and a CDHR3 region comprising SEQ ID NO: 17; and a CDRL1 region comprising SEQ ID NO: 20, a CDRL2 region comprising SEQ ID NO: 21 and a CDRL3 region comprising SEQ ID NO: 22 and sequences at least 90% identical to SEQ ID NO: 15, SEQ ID NO: 16, SEQ ID NO: 17, SEQ ID NO: 10, SEQ ID NO: 11, SEQ ID NO: 12, SEQ ID NO: 20, SEQ ID NO: 21 and SEQ ID NO: 22.

4. The antibody or antigen binding fragment thereof of any of examples 1-3, wherein the heavy chain complementarity-determining region is encoded by SEQ ID NO: 4 and the light chain complementarity-determining region is encoded by SEQ ID NO: 9 and sequences 90% identical to SEQ ID NO: 4 and SEQ ID NO: 9; or the heavy chain complementarity-determining region is encoded by SEQ ID NO: 14 and the light chain complementarity-determining region is encoded by SEQ ID NO:19 and sequences 90% identical to SEQ ID NO: 14 and SEQ ID NO: 19.

5. The antibody of any of the previous examples, wherein the FcγRIIa is on a platelet.

6. The antibody or antigen binding fragment of any of examples 1-5 wherein the antibody is linked to a detectable label.

7. The antibody or antigen binding fragment of any of examples 1-6, wherein the FcγRIIa is on a platelet and the platelet has been fixed with a fixative before binding the antibody to the FcγRIIa.

8. The antibody or antigen binding fragment of any of the previous examples, wherein the antibody is an isolated monoclonal antibody.

9. The antibody or antigen binding fragment of any of the previous examples, wherein the antibody is a monoclonal antibody.

10. The antibody or antigen binding fragment of any of the previous examples, wherein the antibody is or is produced using recombinant antibody technology, nucleic acid aptamer technology or non-immunoglobulin protein scaffold technology.

11. A conjugate of an antibody bound to FcγRIIa, wherein the antibody comprises any antibody or antigen binding fragment of any of the previous examples.

12. A method of detecting FcγRIIa comprising binding FcγRIIa to an antibody, the antibody comprising the antibody or antigen binding fragment of any of the previous examples.

13. A method of detecting the presence of FcγRIIa in a blood sample comprising binding an antibody to FcγRIIa, the antibody comprising the antibody or antigen binding fragment of any of the previous examples.

14. The method of example 13, where the platelets in the blood sample are treated with a fixative prior to or at the same time the antibody is introduced to the blood sample.

15. The method of example 13 or 14, wherein the fixative is combined with the blood sample up to 2 days following collection of the blood sample.

16. The method of any of examples 13-15 wherein the fixative is diluted in a solution comprising a buffer and glycerol prior to combining with a blood sample.

17. The method of any of examples 13-16, wherein the buffer is Phosphate Buffered Saline (PBS), and the glycerol comprises a range of approximately 2% to 7% of the total volume of the buffer-glycerol solution.

18. The method of examples 16 or 17 wherein the glycerol is 5% of the total volume of the buffer-glycerol solution.

19. The method of any of examples 12-18, where the bound FcγRIIa and antibody complex are detected via flow cytometry to measure the level of FcγRIIa in the sample.

20. The method of example 19, where the measured level of FcγRIIa in the sample is standardized based on a comparison of measured levels of fluorescence in samples with known levels of fluorescence markers.

21. A method of detecting the presence of FcγRIIa in a blood sample comprising the steps of:
treating a blood sample with an anticoagulant;
adding a fixative to the blood sample;
separating and washing the platelets from the blood sample;
incubating the platelets with an antibody, the antibody comprising any one of the antibodies or antigen binding fragments of any of examples 1-11; and
performing an analysis to quantify FcγRIIa in the sample.

22. The method of example 21, wherein the analysis is selected from the group consisting of: flow cytometry, immunoassay, ELISA, western blotting, and radioimmunoassay.

23. The method of example 22, wherein the analysis is flow cytometry.

24. The method of any of examples 21-23, further comprising the steps of:
reducing the concentration of the fixative in the blood sample; and
storing the blood sample from a period up to and including 1-14 days before incubating the platelets with the antibody.

25. A method of preparing a blood sample comprising the steps of:
treating a blood sample with an anticoagulant;
adding a fixative to the blood sample;
subsequently diluting the concentration of the fixative in the blood sample; and
storing the blood sample from a period up to and including 1-14 days.

26. The method of example 25, wherein the fixative is added to a concentration of 5% and is subsequently diluted to 1.25%.

27. A method of detecting FcγRIIa comprising binding FcγRIIa to an antibody, the antibody comprising at least one heavy chain complementarity-determining region (CDRH) and at least one light chain complementarity-determining region (CDRL), wherein the CDRH is selected from SEQ ID NO: 5, SEQ ID NO: 6 and SEQ ID NO: 7 and the CDRL are selected from SEQ ID NO: 10, SEQ ID NO: 11 and SEQ ID NO: 12 wherein SEQ ID NO: 11 encodes the amino acid sequence DTS, or wherein the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22 wherein SEQ ID NO: 21 encodes the amino acid sequence WAS.

28. The method of example 27, wherein FcγRIIa is detected on a platelet from a blood sample.

29. The method of example 27 or 28, further comprising comparing the level of detection of FcγRIIa between a blood sample of a human having, or suspected of having heart disease, thrombosis, coronary artery disease, renal disease, or myocardial infarction and a control human blood sample that does not have having heart disease, thrombosis, coronary artery disease, renal disease, or myocardial infarction.

31. The antibody or antigen binding fragment of any of examples 1-11, for use in a method of detecting platelet reactivity in a subject.

All patents and publications mentioned in this specification are herein incorporated by reference to the same extent as if each independent patent and publication was specifically and individually indicated to be incorporated by reference.

From the foregoing description, it will be apparent that variations and modifications may be made to the subject matter described herein to adopt it to various usages and conditions. Such embodiments are also within the scope of the following claims.

The following examples are put forth to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: FcγRIIa is Phosphorylated Following Platelet Activation

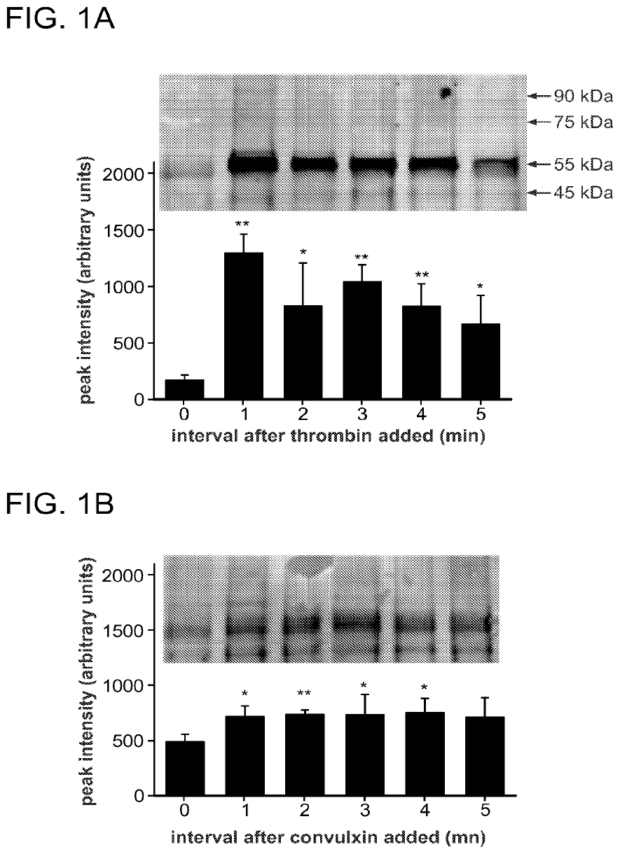

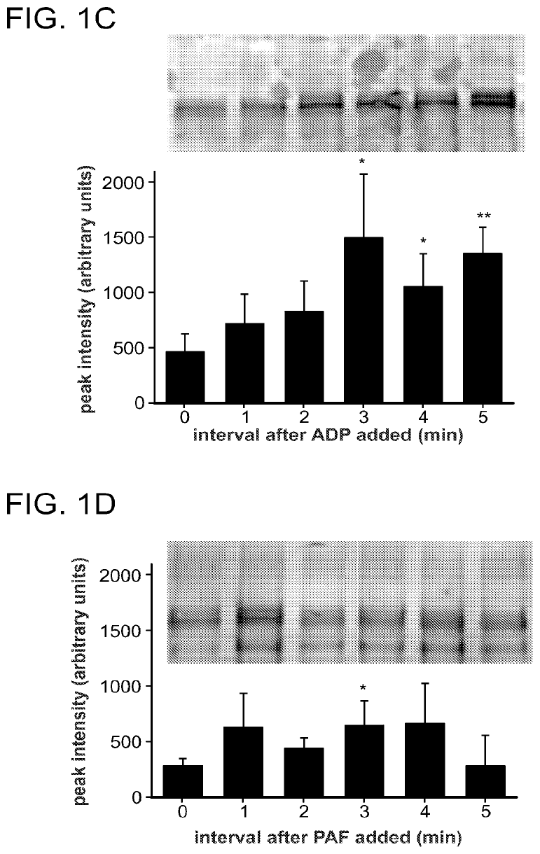

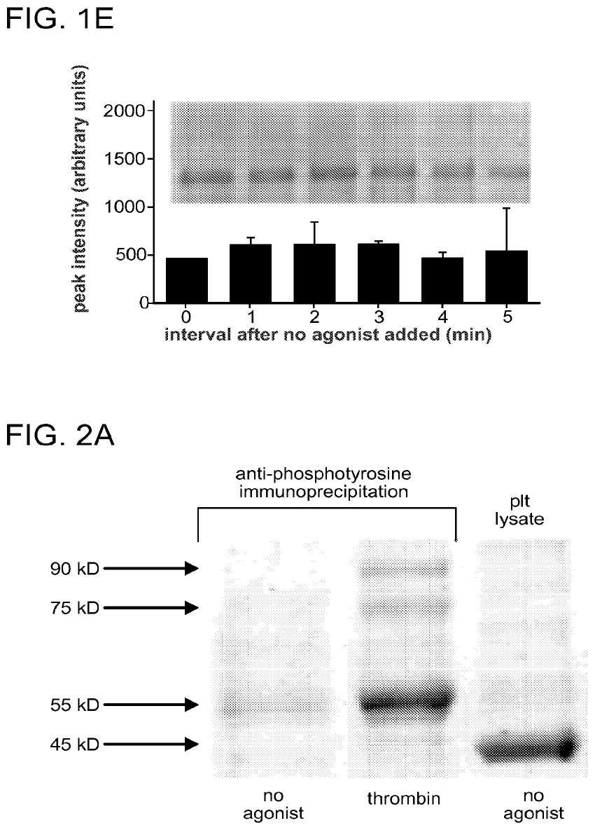

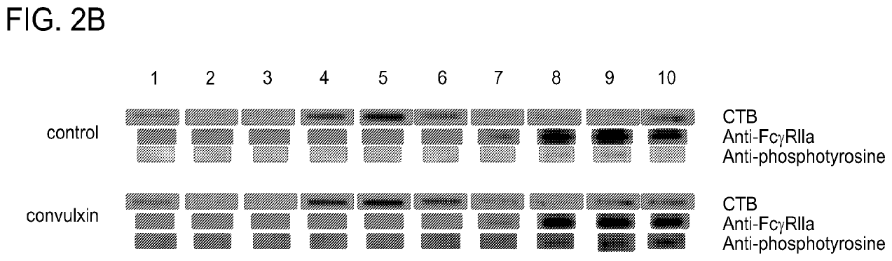

Phosphorylation of immunoprecipitated FcγRIIa following activation with thrombin, convulxin, ADP, and PAF was quantified from platelets isolated from low-risk subjects (FIGS. 1A-1E). Although a band corresponding to the expected molecular weight of FcγRIIa was apparent at 45 kDa, additional bands at 55 kDa, 75 kDa, and 90 kDa demonstrated phosphorylation. In subsequent experiments, immunoprecipitation was performed with the use of an anti-phosphotyrosine antibody (4G10). These gels confirmed that phophorylated FcγRIIa was associated with each of the 4 bands originally identified (FIG. 2A). Phosphorylation of FcγRIIa was greatest early (1-2 min) after exposure to thrombin, convulxin, and PAF whereas maximal effects of ADP were not apparent until later (3-5 min).

Mass spectrometry was used to identify proteins or fragments of proteins that coimmunoprecipitated with FcγRIIa after activation of platelets. As expected, approximately 45% of the protein content of the band at 55 kDa was IgG (used for the immunoprecipation). Fibrinogen (predominantly 0 chain, 33%), actin (3%) and the SRC kinase Lyn (1%) were the proteins co-immunoprecipitated in the 55 kDa band. Without being bound to a particular theory, modest phosphorylation of FcγRIIa is associated with substantial phosphorylation of Lyn. Proteins comprising the 75 kDa band included coagulation Factors XIII (A subunit, 38%) and V (4%), fibrinogen (α, β, and γ chains 24%), nexilin (3%), actin (1%) and myosin (1%). Proteins comprising the 90 kDa band included fibrinogen (γ chain, 80%), gelsolin (6%), filamin (1%), and coagulation Factor XIII (1%). Accordingly, with activation of platelets FcγRIIa was associated predominantly with fibrinogen, coagulation Factor XIII, and constituents of the platelet cytoskeleton (actin, myosin, nexilin, gelsolin, and filamin).

### Example 2: FcγRIIa Co-Localizes with Lipid Rafts with Platelet Activation

Lipid rafts were isolated and extracts from platelets that had not been activated demonstrated FcγRIIa in fractions 8-10, the membrane cytoskeleton (FIG. 2B). After activation, FcγRIIa remained associated with the membrane cytoskeleton (fractions 8-10), phosphorylation of FcγRIIa was seen in fractions 8-10, and plasma membrane lipid rafts were seen in fractions 9 and 10 (FIG. 2B). FcγRIIa was not seen in association with soluble lipid rafts (fractions 4-6).

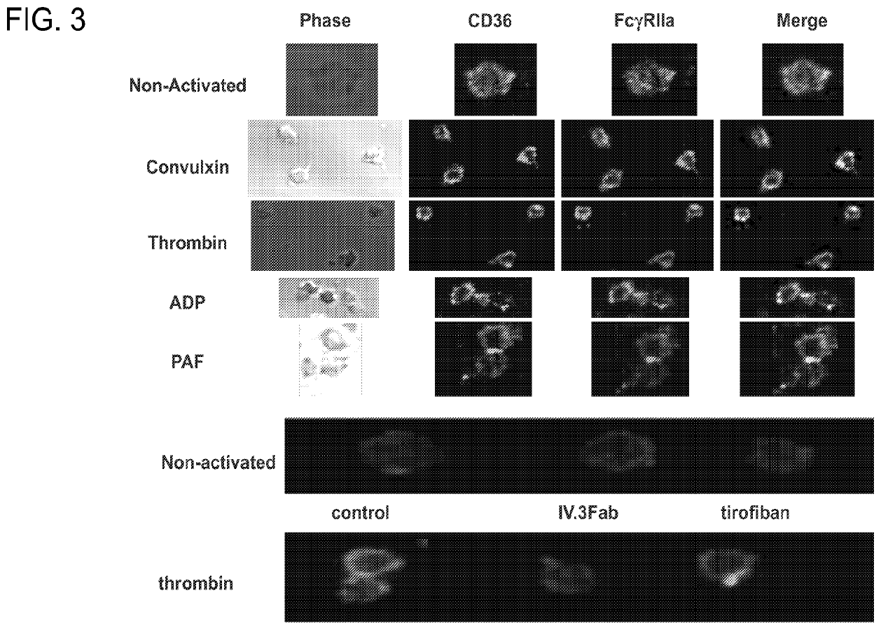

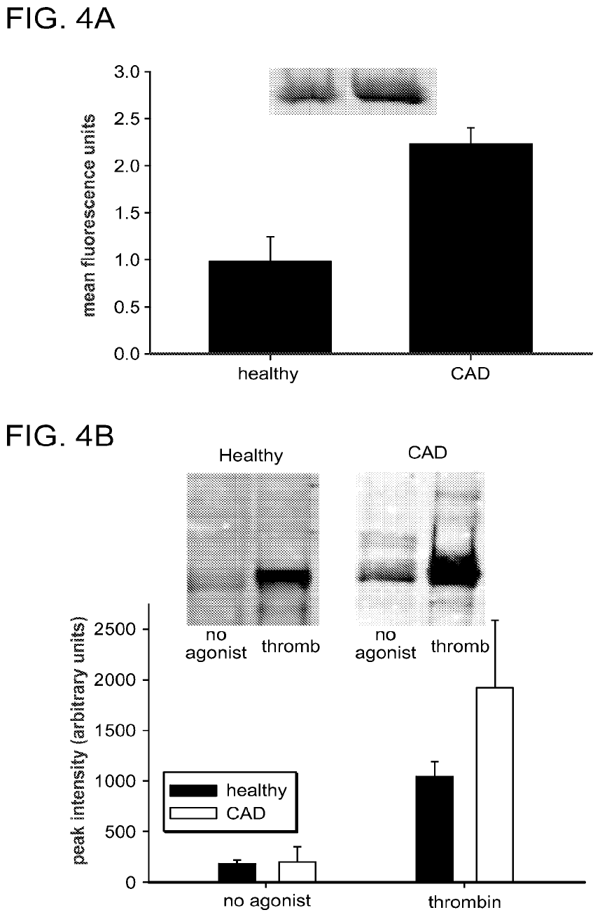

Confocal microscopy demonstrated a homogeneous surface expression of FcγRIIa in the absence of activation and clustering of FcγRIIa after activation (FIG. 3). Consistent with previous results demonstrating that CD36 is confined to the plasma membrane (Roper K, et al., Nat Cell Biol 2000; 2:582-592), nonactivated platelets exhibited a homogeneous surface expression of CD36 and activated platelet exhibited clustering of CD36. Co-localization of lipid rafts with FcγRIIa was greater with activated platelets (FIG. 3).

### Example 3: Inhibition of FcγRIIa Phosphorylation Attenuates Platelet Activation

Patients (n=3) with coronary artery disease (CAD) in whom platelet expression of FcγRIIa was 2-fold greater than the average expression by platelets from low-risk subjects were chosen for inclusion in this study. Expression of FcγRIIa was identified with the use of flow cytometry and confirmed with Western blot analysis (FIG. 4A). Extracts from platelets with greater expression of FcγRIIa exhibited greater phosphorylation of FcγRIIa (by ˜2-fold) after activation with thrombin (FIG. 4B).

Concentrations of IV.3 Fab (100 μg/ml) and the SRC kinase antagonist PP2 (20 μM) that prevented phosphorylation of FcγRIIa during platelet activation were identified. Tirofiban was used to block binding of fibrinogen to GP IIb-IIIa. Goat anti-FcγRIIa cross-links FcγRIIa and was used as a direct acting activator of FcγRIIa.

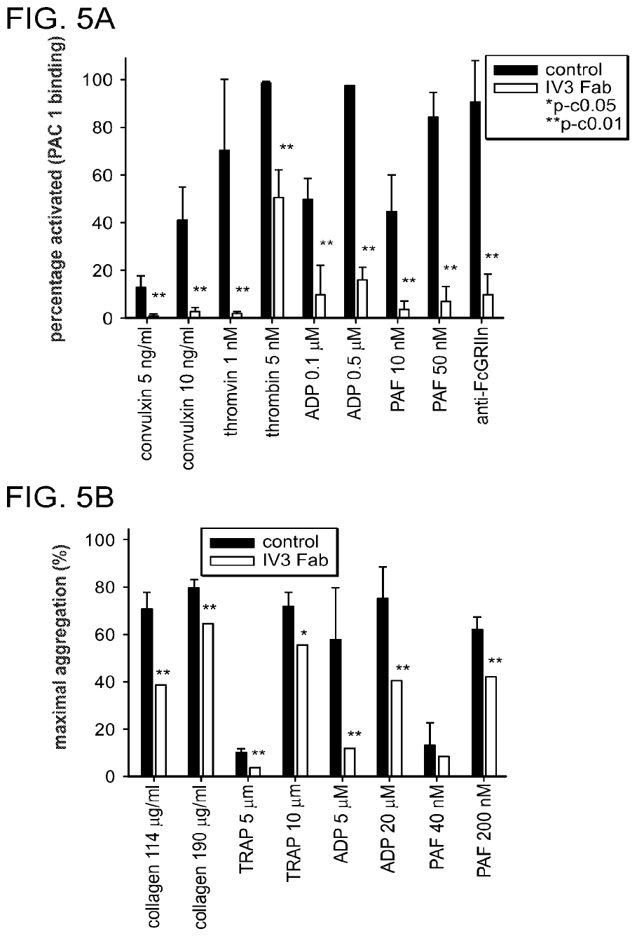

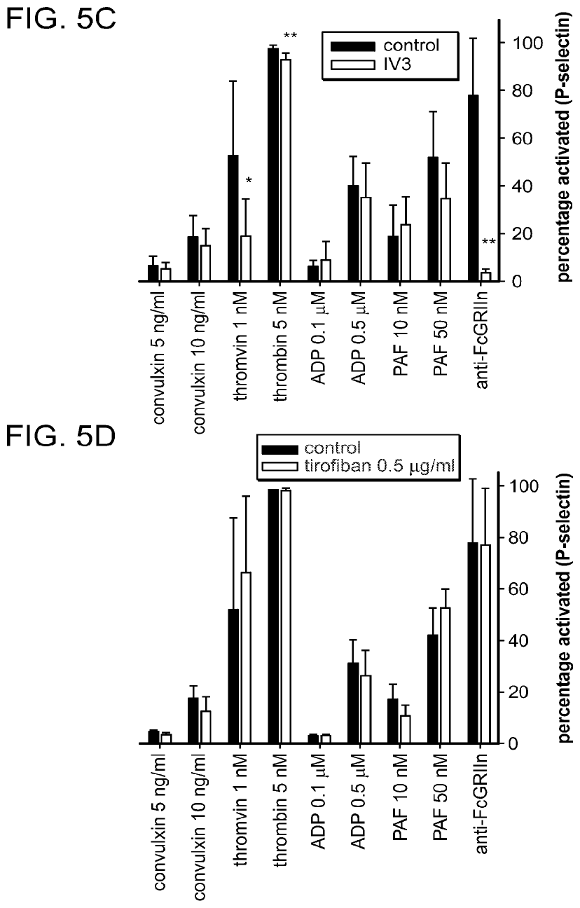

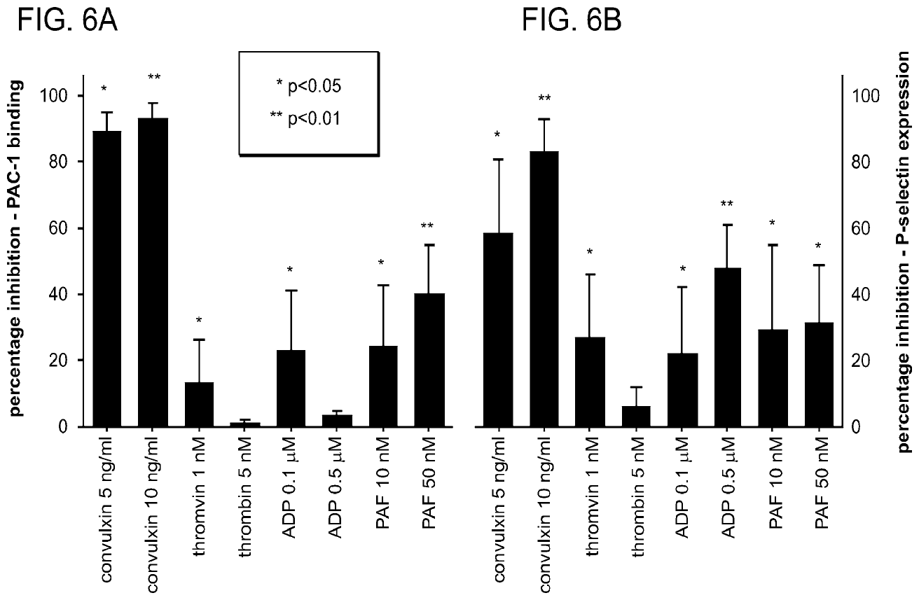

Inhibition of phosphorylation of FcγRIIa by IV.3 attenuated the activation of platelets, particularly when the activation was identified by the binding of PAC-1 (FIGS. 5A & 5B). Results were confirmed with the use of light transmission aggregometry (FIG. 5C). When thrombin or TRAP was the agonist, the extent of inhibition caused by IV.3 was more apparent when lower concentrations of agonist were used (FIG. 5D). As expected, IV.3 effectively blocked the majority of platelet activation induced by goat anti-FcγRIIa. By contrast, a concentration of tirofiban (0.5 μg/ml) that blocked completely the binding of PAC-1 to platelets did not attenuate the activation of platelets induced by any of the agonists nor did it alter activation induced by goat anti-FcγRIIa. Results with confocal microscopy suggested that IV.3 attenuated clustering of FcγRIIa (FIG. 3). Consistent with the results seen with the specific antagonist IV.3, inhibition of SRC kinases by PP2 attenuated the activation of platelets (FIG. 6).

Purified human fibrinogen was added to whole blood from low-risk subjects (to increase the concentration by 500 mg/dl) and the activation of platelets in response to 0.5 μM ADP was assessed by the surface expression of P-selectin. The addition of fibrinogen increased ADP induced activation of platelets (fold induction—1.50.2, n=3). This effect was blocked by pretreatment with IV.3 (100 μg/ml, fold induction—0.90.1, p<0.05) and appeared to be attenuated by tirofiban (0.5 μg/ml, fold induction—1.20.1, p=0.11).

Human coagulation Factor XIII was added to blood from low-risk subjects (to increase the concentration by 1 μM) and the activation of platelets in response to 0.5 μM ADP was assessed by the surface expression of P-selectin. The addition of Factor XIII increased ADP-induced activation of platelets (fold induction—3.90.1, n=3). This effect was blocked by pretreatment with IV.3 (100 μg/ml, fold induction—0.70.1, p<0.001) and by tirofiban (0.5 μg/ml, fold induction—0.80.2, p<0.001).

### Example 4: FcγRIIa and Platelet Activation in Disease States

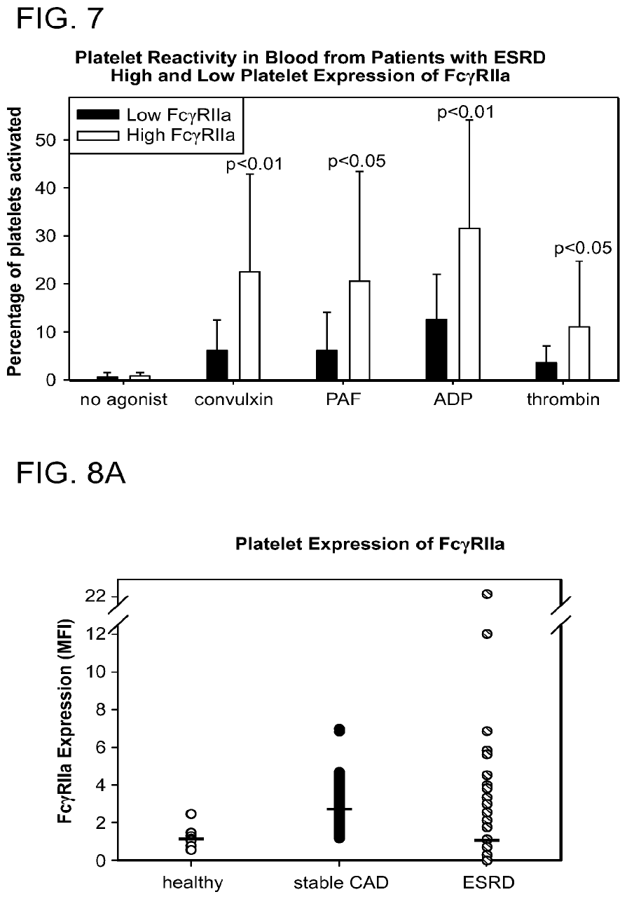

As shown in FIG. 7 platelets derived from patients with end stage renal disease (ESRD) that express high levels of FcγRIIa demonstrated higher percentage of activated platelets than platelets that express lower levels of FcγRIIa.

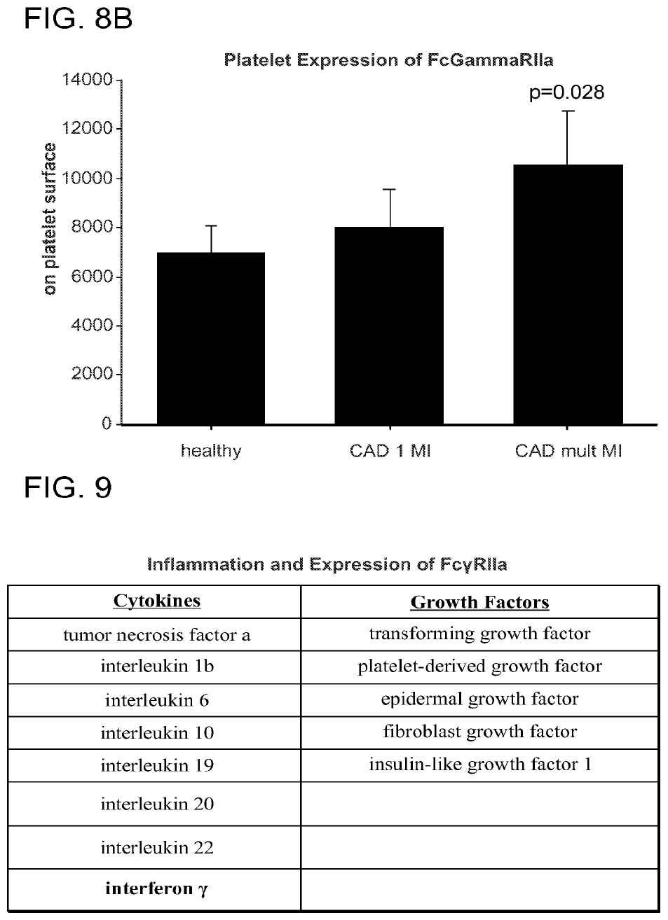

As shown in FIG. 8A, platelets from patients with coronary artery disease (CAD) and end stage renal disease (ESRD) express higher levels of FcγRIIa than platelets from low-risk controls. As shown in FIG. 8B, platelets from patients with coronary artery disease (CAD) and one or more myocardial infarctions express higher levels of FcγRIIa than platelets from low-risk controls. In particular, patients with multiple myocardial infarctions have the highest expression of FcγRIIa and all patients with CAD have higher expression of FcγRIIa.

As shown in FIG. 9, a number of inflammation related cytokines and growth factors influence the expression of FcγRIIa.

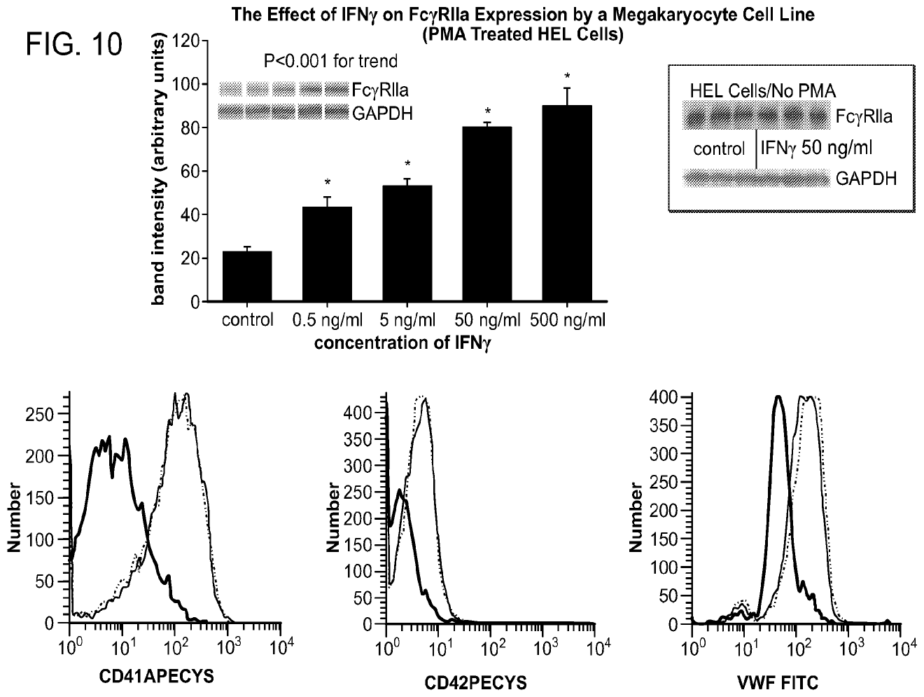

As shown in FIG. 10, IFNγ treatment increases the expression of FcγRIIa by a Megakaryocyte cell line.

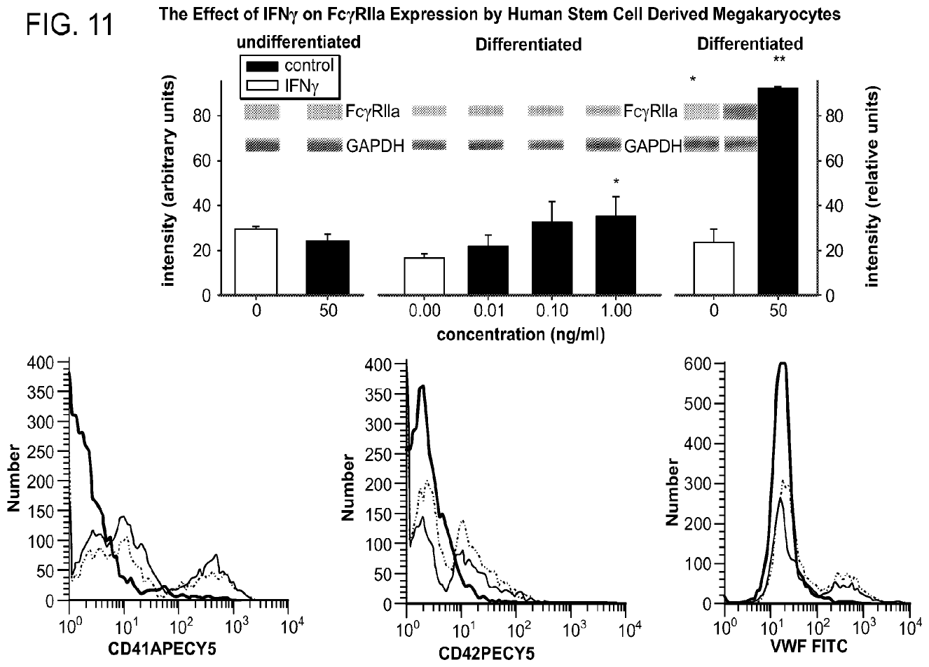

As shown in FIG. 11, IFNγ treatment increases the expression of FcγRIIa by human stem cell derived Megakaryocytes.

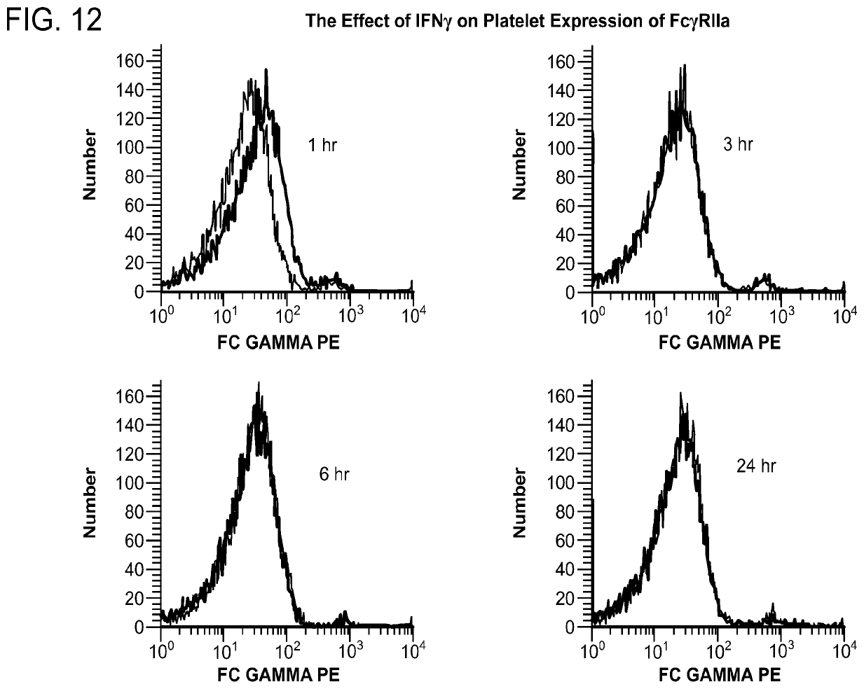

FIG. 12 shows the effect of IFNγ treatment on platelet expression of FcγRIIa.

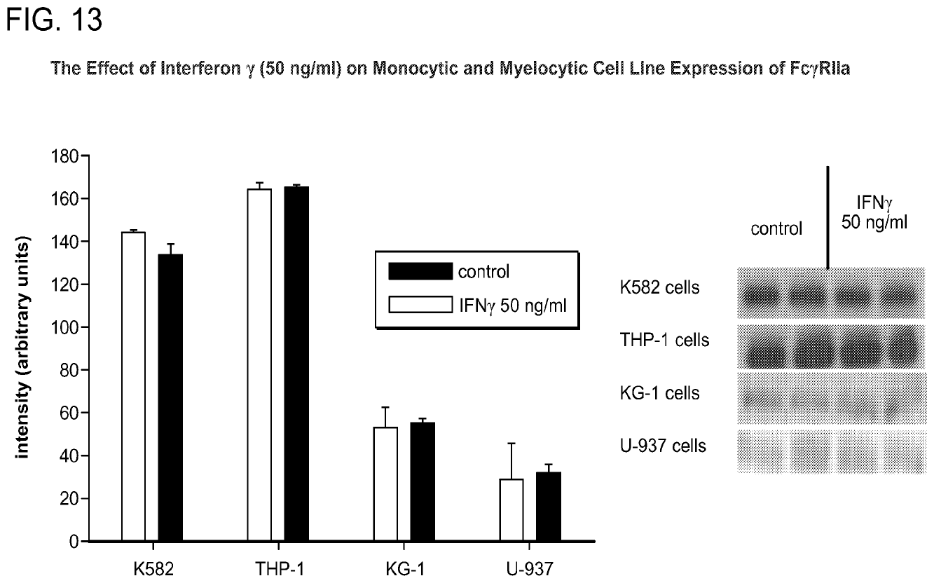

FIG. 13 shows the effect of IFNγ treatment on monocytic and myelocytic cell line expression of FcγRIIa.

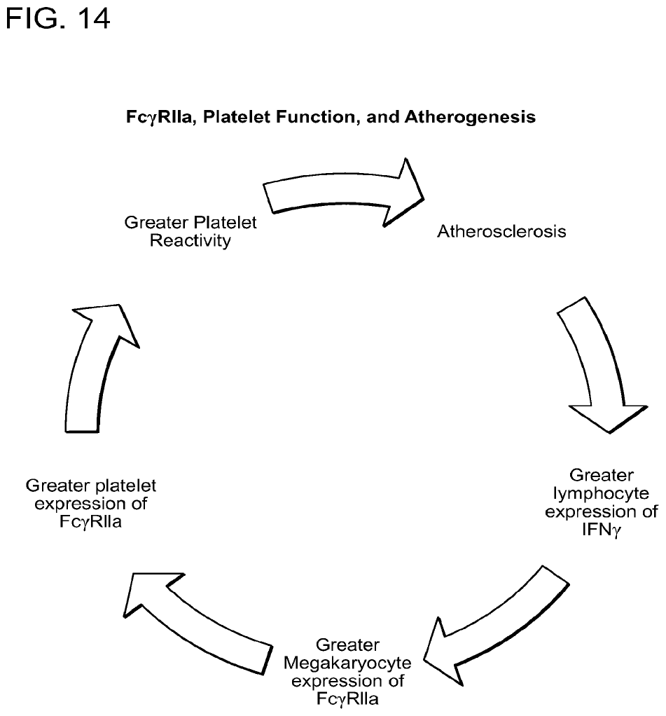

The relationship of FcγRIIa, platelet activation, and atherogenesis is illustrated in FIG. 14.

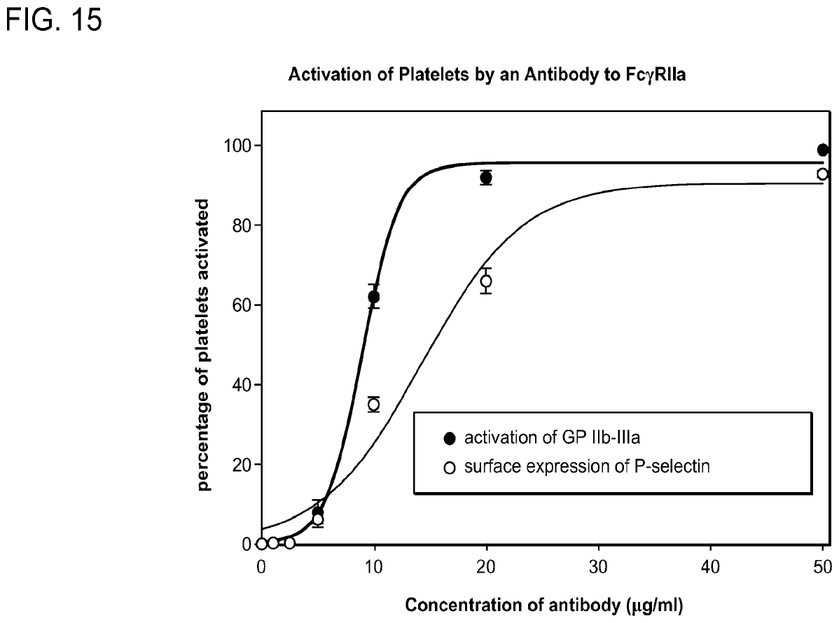

As shown in FIG. 15, an antibody specific for FcγRIIa is able to cause platelet activation.

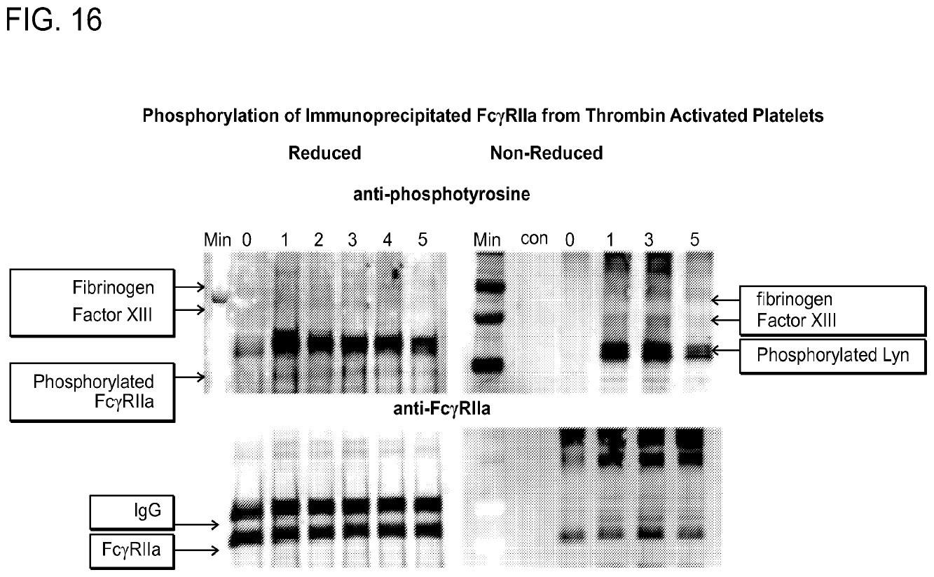

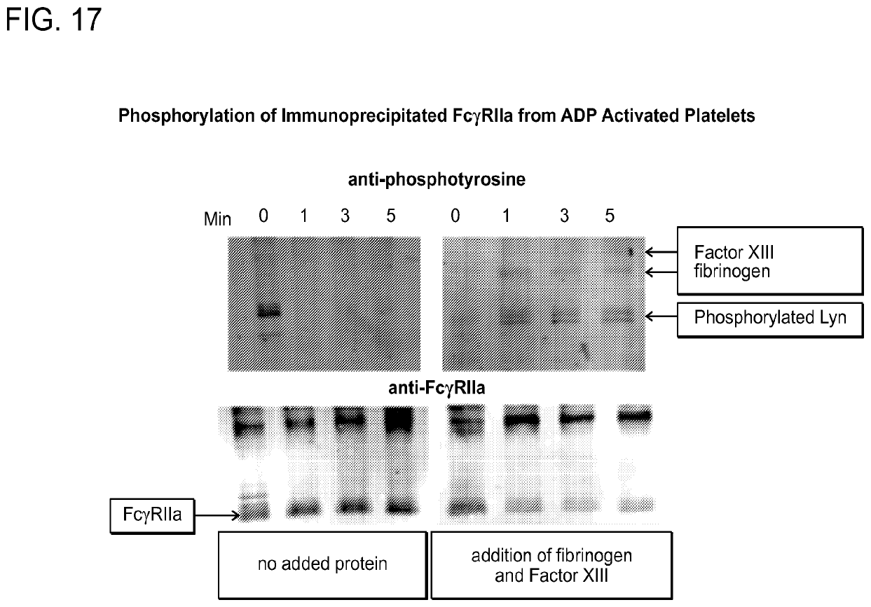

As shown in FIGS. 16 and 17, FcγRIIa becomes phosphorylated in thrombin and ADP activated platelets respectively.

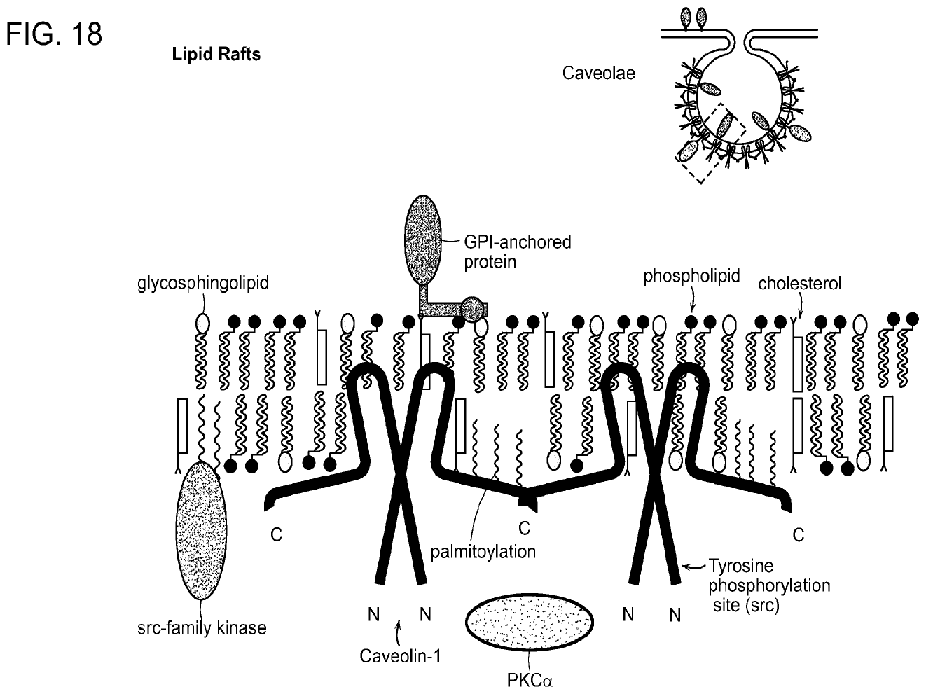

The structure of lipid rafts is illustrated in FIG. 18.

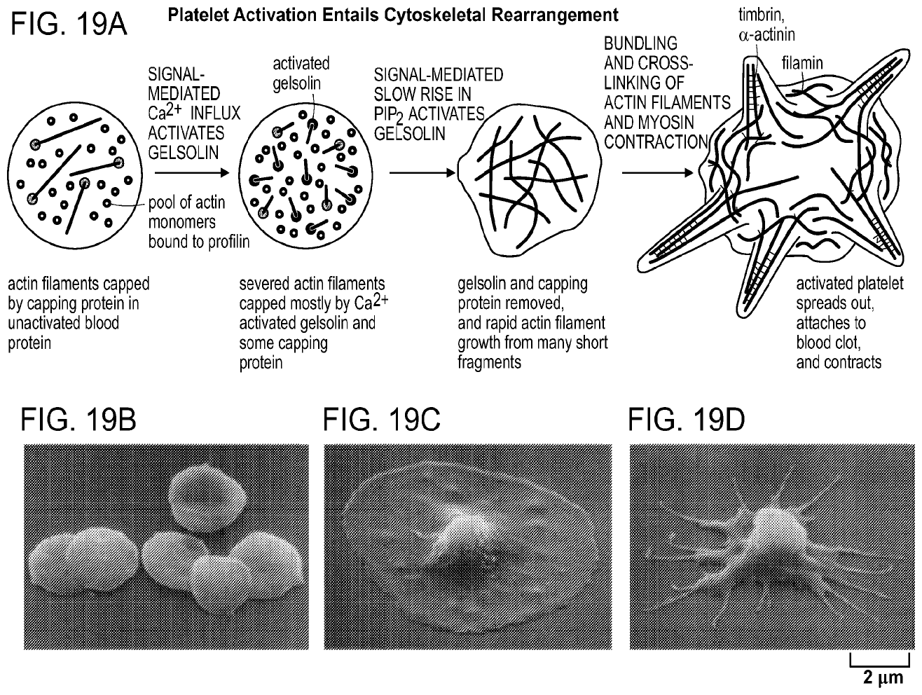

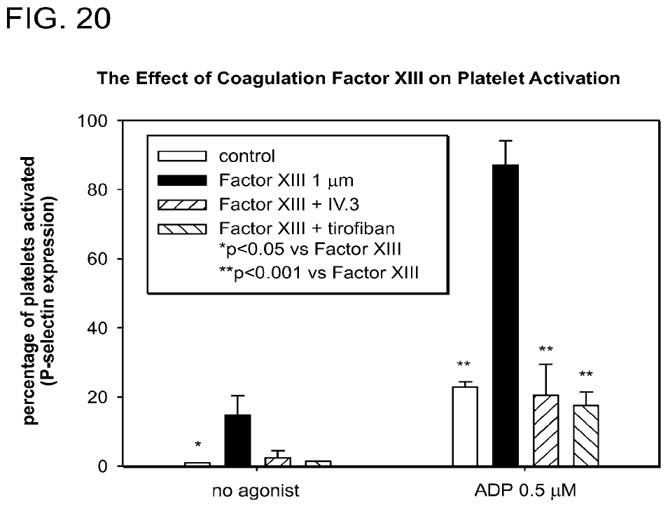

As shown in FIG. 19, platelet activation entails cytoskeletal rearrangement.

FIG. 20 shows the effect of coagulation factor XIII on platelet activation.

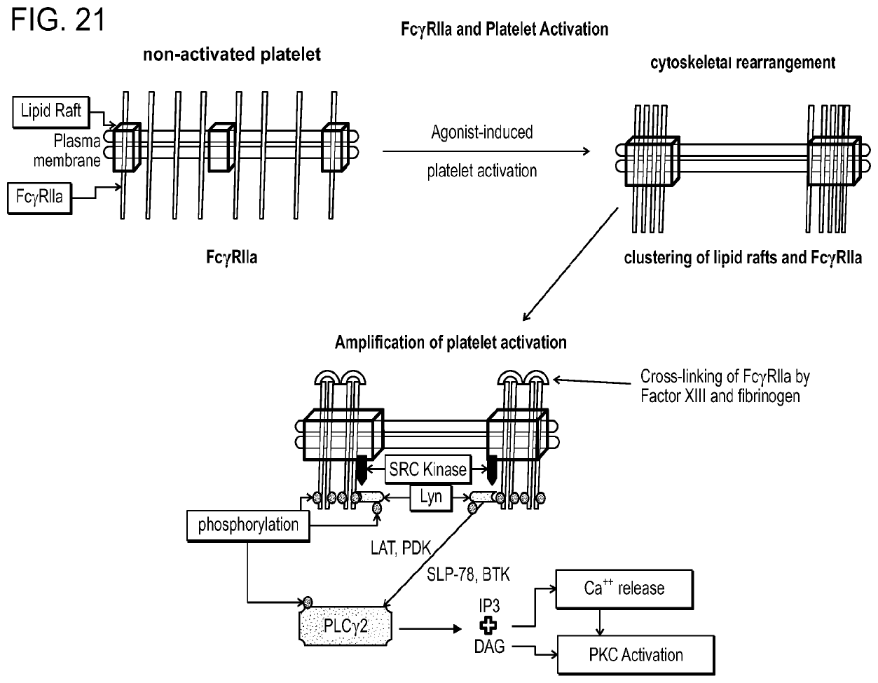

The role of FcγRIIa in platelet activation is illustrated in FIG. 21.

The examples presented in this application were performed using the following materials and methods, except as otherwise indicated.

### Blood Sample Collection

In accordance with a protocol approved by the University of Vermont Institutional Review Board, blood was taken from low-risk subjects or from patients with coronary artery disease after they provided written informed consent. Patients with coronary artery disease had a previous myocardial infarction or coronary revascularization plus elevated platelet expression of FcγRIIa and were treated with aspirin but not other antiplatelet or anticoagulant medication. Phlebotomy was performed with a 21 gauge butterfly needle, tourniquets were applied for less than 90 seconds, and the first 3 ml of blood were discarded. Blood (1 ml) for assay of platelet function was anticoagulated with 32 μg/ml of corn trypsin inhibitor (CTI, Haematologic Technologies Inc, Essex Junction, Vt.), a specific inhibitor of Factor XIIa without effect on other coagulation factors (Rand M D, et al., Blood 1996; 88:3432-45), that does not alter the activation of platelets (Schneider D J, et al., Circulation 96:2877-83, 1997). Washed platelets were prepared from blood anticoagulated with acid citrate dextrose (ACD, trisodium citrate, 0.085 M; citric acid, 0.071 M; glucose 0.1 M, pH 4.5, 1:10 v/v).

### Assessment of Platelet Function:

To assess platelet function, 5 μl aliquots of whole blood were added to tubes containing HEPESTyrodes (HT) buffer (5 mM HEPES, 137 mM NaCl, 2.7 mM NaHCO₃, 0.36 mM NaH2PO4, 2 mM CaCl2, 4 mM MgCl2, and 5 mM glucose, pH 7.4) and fluorochrome-labeled ligands. Volumes used minimize aggregation of platelets during activation. Activation of platelets was identified by anti-CD62-phycoerythrin (PE, identifies P-selectin) and fluorescein isothiocyanate (FITC)-conjugated PAC-1 (binds to activated GP IIb-IIIa) as described (Serrano F A, et al., Thromb J 2007; 5:7; Schneider D J, et al., Circulation 96:2877-83, 1997; Kabbani S S, et al., Circulation 2001; 104:181-6; Aggarwal A, et al., Am J Kidney Dis 2002; 40:315-22; and Schneider D J, et al., Diabetes Care, 32:944-9, 2009). Platelet expression of FcγRIIa was quantified with the use of anti-CD32-PE. For both assays (activation and quantification of expression of FcγRIIa) PE-Cy5-anti-CD42b was used as an activation independent marker of platelets. When thrombin or coagulation Factor XIII were used, the peptide GPRP (Gly-Pro-Arg-Pro) was added to prevent polymerization of fibrinogen (Achyuthan K E, et al., Biochim Biophys Acta 1986; 872:261-8). Fluorochrome labeled antibodies were from Becton Dickinson (San Jose, Calif.). Human α-thrombin and coagulation Factor XIII were from Haematologic Technologies Inc (Essex Junction, Vt.), PAF from EMD Biosciences (Gibbstown, N.J.), fibrinogen (>95% pure) from Sigma (St. Louis, Mo.), and ADP from BioData (Horsham, Pa.). Convulxin is a collagen-mimetic lectin that binds to GP VI (Clemetson J M, et al., J Biol Chem 1999; 274:29019-24, Pentapharm, Basel, Switzerland). The Fab of an antagonist to FcγRIIa (IV.3) was prepared (Pierce Fab preparation kit, Thermo Scientific, Rockford, Ill.) from IgG produced by hybridoma cells (HB-217 cells, American Tissue Culture Center, Manassas, Va.) (Looney, R. J. et al., J Exp Med 1986; 163:826-836). The SRC kinase antagonist PP2 (EMD Biosciences) was dissolved in dimethylsulfoxide (DMSO, Sigma). Tirofiban was obtained from Merck Research Laboratories (Whitehouse Station, N.J.).

The reaction mixture was incubated for 15 minutes at room temperature without stirring before platelets were fixed and erythrocytes lysed by addition of Optilyse-C solution (Beckman Coulter). Flow cytometric analysis was performed with the use of a Beckman Coulter FC500 (Miami, Fla.). Platelets were identified on the basis of size (forward and side scatter) and the binding of an activation independent ligand (anti-CD42b). As described, control samples to define a threshold for activation-dependent binding used non-immune IgG (Serrano F A, et al., Thromb J 2007; 5:7; Schneider D J, et al., Circulation 96:2877-83, 1997; Kabbani S S, et al., Circulation 2001; 104:181-6; Aggarwal A, et al., Am J Kidney Dis 2002; 40:315-22; and Schneider D J, et al., Diabetes Care, 32:944-9, 2009). Activation of platelets is reported as the percentage of platelets that bound an activation dependent ligand, a result that we have shown correlates directly with mean fluorescence intensity (Schneider D J, et al., Thromb Haemost 2001; 85:309-13).

Turbidometric platelet aggregation was performed with the use of a PAP-4 aggregometer (BioData, Horsham Pa.). Maximal aggregation after 4 min was reported. For aggregometry, thrombin receptor agonist peptide (TRAP, Bachem, Torrance, Calif.) was to mimic effects of thrombin.

### Assessment of Platelet Proteins:

Washed platelets (2×10⁸ in 0.5 ml) isolated by gel filtration (Sepharose CL-2B, Sigma) from platelet rich plasma (PRP-centrifugation of 140 g×15 min at room temperature) used to demonstrate phosphorylation of FcγRIIa were activated at room temperature without stirring for selected intervals. Whole platelet lysates were prepared by the addition of an equal volume of 2× lysis buffer (2% Nonidet P-401, 300 mM NaCl, 50 mM Tris, 2 mM Na3VO4, halt protease and phosphatase inhibitor cocktail [Pierce Biochemicals], pH 7.3) in an ice bath for 30 min. Lysates to be used for immunoprecipitation were pre-cleared of antibodies by the addition of protein G Dynabeads (Invitrogen, Carlsbad, Calif.).

Immunoprecipitation was performed overnight at 4° C. with the use of goat anti-CD32A/C (Santa Cruz Biotechnology, Santa Cruz, Calif.) or a mouse anti-phosphotyrosine conjugated with magnetic beads (4G10, Millipore, Billerica, Mass.). When anti-CD32A/C was used, antigen antibody complexes were isolated with the use of protein G-coated magnetic beads (Dynabeads, Invitrogen, Carlsbad, Calif.) for 3 hours. Beads with antigen-antibody complexes were washed once with 0.5× lysis buffer and twice with phosphate buffered saline. Proteins were separated from beads in sample buffer before electrophoresis and wet transfer to Immobilon FL membranes (Millipore, Billerica, Mass.). Phosphorylation was identified with a mouse anti-phosphotyrosine (clone 4G10, Millipore) or with goat anti-CD32A/C (Santa Cruz Biotechnology, Santa Cruz, Calif.). Bands were detected with the use of use of a Li-Cor Odyssey Infrared Imaging system (Li-Cor Biosciences, Lincoln, Nebr.) or chemiluminescence (Amersham/General Electric Healthcare, Piscataway, N.J.).

Lipid rafts were isolated as described (Lee F A, et al., J Biol Chem 2006; 281:39330-8) from washed platelets (4×10⁸ in 0.5 ml) that were activated for 90 sec at room temperature without stirring and then lysed by the addition of 2× lipid raft lysis buffer (20 mM Tris, 100 mM NaCl, 60 mM sodium pyrophosphate, 20 mM sodium glycerophosphate, 0.02% w/v sodium azide, 0.025 Triton X-100, 2 mM sodium vanadate protease inhibitor tablet, pH 8.0). After 30 min on ice, samples were mixed with equal volumes of 80% w/v sucrose and a sucrose gradient was prepared by addition of 5 ml of 36% w/v sucrose followed by 5 ml of 5% w/v. Each sucrose solution contained 0.025% w/v Triton X-100 and separation was accomplished by centrifugation (200,000 g for 18 hr at 4° C.). Sequential 1 ml fractions were collected.

Proteomic assessment of proteins co-immunoprecipitated with FcγRIIa was performed by the University of Vermont Proteomics Core (Dwight Matthews, Co-Director). Bands visible after GelCode staining (Pierce Biotechnology, Rockford, Ill.) were excised, trypsin digested (ProteaseMAX protocol, Promega, Madison, Wis.) and identified with the use of liquid chromatography-mass spectrometry. High pressure liquid chromatography (Shimadzu, Columbia, Md.) was used to separate protein digests on a 100 μm×50 mm column packed with Michrom 3μ C18 AQ (Auburn, Calif.). Peptides eluted from the column were analyzed with the use of a linear ion trap mass spectrometer (Thermo Scientific, San Jose, Calif.) fitted with a Michrom Advance electrospray source. Proteins were identified from peptide amino acid sequences that were determined using the Thermo Scientific SEQUEST algorithm.

### Confocal Microscopy

Confocal microscopy was performed as described previously (Schneider D J, et al., J Am Coll Cardiol 1999; 33:261-6). Platelets exposed to selected conditions were fixed with Optilyse-C for 15 minutes and then pretreated with 1% bovine serum albumin (BSA) in HT buffer for 15 min before incubation with primary antibodies. A primary goat anti-FcγRIIa/CD32a was used to identify FcγRIIa and anti-CD36 was used to identify lipid rafts (Gousset K, et al., J Cell Physiol 2002; 190:117-28). CD36 was chosen to identify lipid rafts because it is differentially associated with lipid rafts located in the platelet plasma membrane (Gousset K, et al., J Cell Physiol 2002; 190:117-28). Platelets separated by centrifugation (1,500 g×10 min) were washed 3 times (HT) before addition of secondary antibodies to identify CD36 (Alexa 488 anti-mouse IgG) and FcγRIIa (Alexa 555 anti-goat IgG). After centrifugation (1,500 g×10 min), platelets were resuspended and applied to a glass microscopic slide for 30 minutes before 2 washes (HT). A cover slip was applied and platelets were imaged with the use of a Zeiss LSM 510 META confocal/scanning laser microscope (Zeiss Microimaging, Thornwood, N.Y.). Control slides with primary antibody alone and secondary antibody alone were used to identify auto fluorescence and non-specific association of secondary antibodies.

### Statistical Analysis

Results are means±standard deviation. Significance of differences was assessed with the use of Student's t tests. Significance was identified by p<0.05.

### FcγRIIa as a Biomarker to Identify High and Low Cardiovascular Risk

A single center prospective trial was conducted to assess the implications of quantifying platelet expression of FcγRIIa. Quantification of platelet expression of FcγRIIa was performed on citrate anticoagulated whole blood. A 2 μl aliquot of blood was added to a tube with two antibodies—one antibody to identify platelets and one antibody to identify FcγRIIa. Samples were then fixed and red blood cells lysed. After dilution sufficient to create a single particle stream, the samples were analyzed with the use of flow cytometry. The output (mean fluorescence intensity) was standardized to quantify molecules of FcγRIIa/platelet.

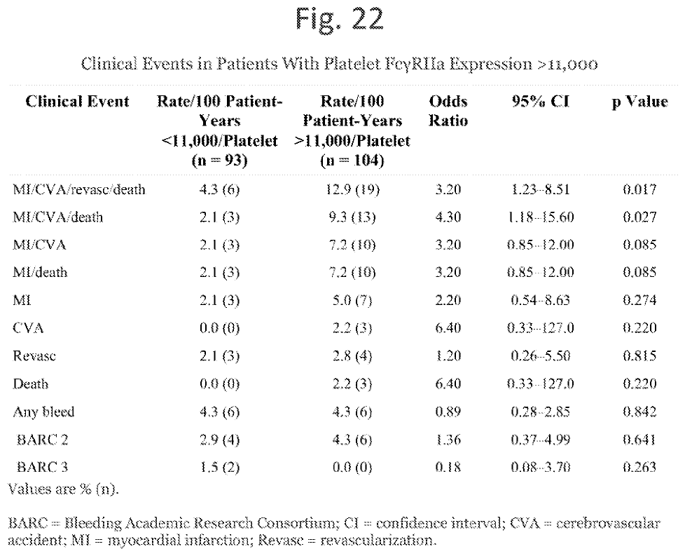

The prospective study was conducted to determine whether platelet expression of FcγRIIa would identify patients with high and low cardiovascular risk (Schneider D J, McMahon S R, Chava S, et al. FcγRIIa: A New Cardiovascular Risk Marker. J Am Coll Cardiol. 2018; 72(2):237-238). Patients (n=197) were enrolled shortly before discharge following a hospitalization for heart attack (both ST elevation and non-ST elevation were included). All patients were treated with ASA (81 mg) and treatment with clopidogrel (^{˜}64%) and ticagrelor (^{˜}36%) was balanced in patients with high and low platelet expression of FcγRIIa. Clinical characteristics were well balanced with the exception of older age, diabetes, and prior revascularization being more prominent in the high expression group. Referring to FIG. 22, the primary endpoint, a composite of heart attack, stroke, death, and coronary revascularization, was lower in patients with platelet expression of FcγRIIa <11,000.

Cox multivariate analysis for the combination of heart attack, stroke revascularization, and death demonstrated a hazard ratio of 3.0 (p=0.02) for platelet expression of FcγRIIa >11,000 when age, diabetes, and prior revascularization were included as covariates. For the combined endpoint of heart attack, stroke and death, Cox regression analysis demonstrated that platelet expression of FcγRIIa was the sole covariate (hazard ratio 3.9, p=0.035) associated with an increased risk of MI, stroke, and death.

The prospective study demonstrated the power of this biomarker to improve care and bridge a critical gap by providing clinicians with a precision tool capable of effectively guiding individualized care. To realize that potential, the test was refined to markedly reduce potential sources of variation. In the original assay, platelets were exposed to antibody and then fixed. A primary objective of the refinement was to allow for platelets to be fixed first and then exposed to antibody. Fixed platelets can be stored and isolated from blood to enhance analytical specificity. To achieve this end, the antibodies disclosed herein were developed to bind to FcγRIIa on the surface of platelets that have been previously fixed.

1. An isolated antibody or antigen binding fragment thereof that specifically binds to FcγRIIa comprising at least one heavy chain complementarity-determining region (CDRH) and at least one light chain complementarity-determining region (CDRL),
wherein the CDRH is selected from SEQ ID NO: 5, SEQ ID NO: 6 and SEQ ID NO: 7 and the CDRL is selected from SEQ ID NO: 10, SEQ ID NO: 11 and SEQ ID NO: 12 wherein SEQ ID NO: 11 encodes the amino acid sequence DTS, or sequences with at least 90% identity to SEQ ID NO: 5, SEQ ID NO: 6, SEQ ID NO: 7, SEQ ID NO. 10, SEQ ID NO. 11 and SEQ ID NO: 12, or;
wherein the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22 wherein SEQ ID NO: 21 encodes the amino acid sequence WAS or sequences with at least 90% identity to SEQ ID NO: 15, SEQ ID NO: 16, SEQ ID NO: 17, SEQ ID NO: 20, SEQ ID NO: 21 and SEQ ID NO: 22.

2. (canceled)

3. (canceled)

4. The antibody or antigen binding fragment thereof of claim 1, wherein
a. the heavy chain complementarity-determining region is encoded by SEQ ID NO: 4 and the light chain complementarity-determining region is encoded by SEQ ID NO: 9 or sequences with at least 90% identity to SEQ ID NO: 4 and SEQ ID NO: 9; or
b. the heavy chain complementarity-determining region is encoded by SEQ ID NO: 14 and the light chain complementarity-determining region is encoded by SEQ ID NO:19 or sequences with at least 90% identify to SEQ ID NO: 14 and SEQ ID NO: 19.

5. The antibody of claim 1, wherein the FcγRIIa is on a platelet.

6. The antibody or antigen binding fragment of claim 5 wherein the antibody is linked to a detectable label.

7. The antibody or antigen binding fragment of claim 6, wherein the FcγRIIa is on a platelet and the platelet has been fixed with a fixative before binding the antibody to the FcγRIIa.

8. (canceled)

9. (canceled)

10. (canceled)

11. A conjugate of an antibody bound to FcγRIIa, the antibody of claim 6.

12. A method of detecting FcγRIIa comprising binding FcγRIIa to an antibody, the antibody comprising the antibody or antigen binding fragment of claim 6.

13. A method of detecting the presence of FcγRIIa in a blood sample comprising binding an antibody to FcγRIIa, the antibody comprising the antibody or antigen binding fragment of claim 6.

14. The method of claim 13, where the platelets in the blood sample are treated with a fixative prior to or at the same time the antibody is introduced to the blood sample.

15. The method of claim 13, wherein the fixative is combined with the blood sample up to 2 days following collection of the blood sample.

16. The method of claim 14 wherein the fixative is diluted in a solution comprising a buffer and glycerol prior to combining with a blood sample wherein the buffer is Phosphate Buffered Saline (PBS) and the glycerol comprises a range of approximately 2% to 7% of the total volume of the buffer-glycerol solution.

17. (canceled)

18. (canceled)

19. The method of claim 16, where the bound FcγRIIa and antibody complex are detected via flow cytometry to measure the level of FcγRIIa in the sample and wherein the measured level of FcγRIIa in the sample is standardized based on a comparison of measured levels of fluorescence in samples with known levels of fluorescence markers.

20. (canceled)

21. A method of detecting the presence of FcγRIIa in a blood sample comprising the steps of:
treating a blood sample with an anticoagulant;
adding a fixative to the blood sample;
separating and washing the platelets from the blood sample;
incubating the platelets with an antibody, the antibody comprising any one of the antibodies or antigen binding fragments of claim 6; and
performing an analysis to quantify FcγRIIa in the sample wherein the analysis is selected from the group consisting of: flow cytometry, immunoassay, ELISA, western blotting, and radioimmunoassay.

22. (canceled)

23. (canceled)

24. The method of claim 21, further comprising the steps of:
reducing the concentration of the fixative in the blood sample; and
storing the blood sample from a period up to and including 1-14 days before incubating the platelets with the antibody.

25. A method of preparing a blood sample comprising the steps of:
treating a blood sample with an anticoagulant;
adding a fixative to the blood sample;
subsequently diluting the concentration of the fixative in the blood sample; and
storing the blood sample from a period up to and including 1-14 days wherein the fixative is added to a concentration of 5% and is subsequently diluted to 1.25%.

26. (canceled)

27. A method of detecting FcγRIIa comprising binding FcγRIIa to an antibody, the antibody comprising at least one heavy chain complementarity-determining region (CDRH) and at least one light chain complementarity-determining region (CDRL), wherein the CDRH is selected from SEQ ID NO: 5, SEQ ID NO: 6 and SEQ ID NO: 7 and the CDRL are selected from SEQ ID NO: 10, SEQ ID NO: 11 and SEQ ID NO: 12 wherein SEQ ID NO: 11 encodes the amino acid sequence DTS, or wherein the CDRH is selected from SEQ ID NO: 15, SEQ ID NO: 16 and SEQ ID NO: 17 and the CDRL is selected from SEQ ID NO: 20, SEQ ID NO: 21 and SEQ ID NO: 22 wherein SEQ ID NO: 21 encodes the amino acid sequence WAS.

28. The method of claim 27, wherein FcγRIIa is detected on a platelet from a blood sample.

29. The method of claim 28, further comprising comparing the level of detection of FcγRIIa between a blood sample of a human having, or suspected of having heart disease, thrombosis, coronary artery disease, renal disease, or myocardial infarction and a control human blood sample that does not have having heart disease, thrombosis, coronary artery disease, renal disease, or myocardial infarction.

30. The method of claim 27, wherein the method detects platelet reactivity.

31. The antibody or antigen binding fragment of claim 1, for use in a method of detecting platelet reactivity in a subject.
